(19) United States
(12) Patent Application Publication
(10) Pub. No.: US 2024/0401069 A1
(43) Pub. Date: Dec. 5, 2024

(54) Production of Milk Oligosaccharides in Plants

(71) Applicant: The Regents of the University of California, Oakland, CA (US)

(72) Inventors: Patrick M. Shih, Berkeley, CA (US); Collin Robert E. Barnum, Berkeley, CA (US)

(73) Assignee: The Regents of the University of California, Oakland, CA (US)

(21) Appl. No.: 18/805,564

(22) Filed: Aug. 15, 2024

Related U.S. Application Data
(63) Continuation of application No. PCT/US23/63730, filed on Mar. 3, 2023.
(60) Provisional application No. 63/316,945, filed on Mar. 4, 2022.

(51) Int. Cl. C12N 15/82; C12N 9/10; C12N 9/88; C12N 15/52

(52) U.S. Cl. CPC C12N 15/8243; C12N 9/1051; C12N 9/88; C12N 15/52; C12Y 204/01069; C12Y 204/01274; C12Y 402/01047

(57) ABSTRACT

Mammalian milk oligosaccharides (MMO) are produced in plants engineered to express recombinant MMO biosynthetic pathways.

### CROSS-REFERENCES TO RELATED APPLICATIONS

This application is a continuation of PCT/US23/63730, filed Mar. 3, 2023, which claims priority to U.S. Provisional Application No. 63/316,945, filed Mar. 4, 2022, the disclosures of which are hereby incorporated by reference in its entirety for all purposes.

### GOVERNMENT SUPPORT CLAUSE

This invention was made with government support under Department of Energy award DEAC02-05CH11231. The government has certain rights in the invention.

### REFERENCE TO A SEQUENCE LISTING

A Sequence Listing in XML format is incorporated by reference into the specification. The name of the XML file containing the Sequence Listing is B22-088-3US.xml. The XML file is 83,560 bytes and was created on Aug. 24, 2024.

### INTRODUCTION

Human breast milk is a complete and comprehensive food perfectly evolved to nourish and protect infants. A key component to the distinct bioactive properties of breast milk is the presence of a wide diversity of human milk oligosaccharides (HMOs) that are well documented in establishing the nascent gut microbiota of infants to prevent diseases and ensure healthy development. Although commercial infant formulas have tried to replicate the biochemical properties of breast milk, these synthetic cocktails are unable to recapitulate the unique health benefits of breast milk due to an inability to produce the diversity of HMOs found in human milk at scale.

Currently, HMOs for infant formula have been largely produced via fermentation using engineered microbes; however, this approach is expensive and has been limited to the production of a small number of simple HMOs. Thus, there is great interest in developing new production hosts for HMOs. As such, plants serve as an attractive production platform, as they have an innate ability to generate a vast array of sugars and use them to produce complex oligosaccharide and polysaccharides. All HMOs can be grouped into three broad classes: neutral, fucosylated, and acidic. Here, we engineer plants to produce diverse sets of all three classes of HMOs (FIG. 1A). This includes the production of HMOs via transient expression and expression in stably transformed plants. Additionally, we increased accumulation of HMOs in plants through modulating nucleotide sugar pathways (FIG. 1B) to optimize the pool of substrate needed for HMO production. Finally, we demonstrate how plants can be used to make complex HMOs that have never before been produced through heterologous production, highlighting the advantages of engineering plants as an ideal production platform of HMOs.

### SUMMARY OF THE INVENTION

For this invention we chose plants serve as a production platform, as they have an innate ability to generate a vast array of sugars and use them to produce complex oligosaccharide and polysaccharides. Mammalian milk oligosaccharide (MMOs) can be grouped into three broad classes: neutral, fucosylated, and acidic. We engineered plants to produce diverse sets of all three classes of MMOs (FIG. 1A). Additionally, we increased accumulation of MMOs in plants through modulating nucleotide sugar pathways to optimize the pool of substrate needed for MMO production (FIG. 1B). Finally, we demonstrated how plants can be used to make complex MMOs that have never before been produced through heterologous production. Our approach allows the production of complex human milk oligosaccharides at an agricultural scale, permitting high production. This enables the supplementation of infant formula with complex MMOs, provide researchers with complex MMOs to conduct research on their bioactivity, and treat diseases in adults.

The invention provides methods and compositions for plant production of mammalian (e.g. human) milk oligosaccharides.

In an aspect the invention provides a method of producing a mammalian milk oligosaccharide (MMO) in a plant, comprising growing a plant comprising and expressing a recombinant MMO biosynthetic pathway sufficient to produce the MMO.

In an aspect the invention provides a composition comprising a plant or plant part comprising and expressing a recombinant MMO biosynthetic pathway sufficient to produce the MMO.

In an aspect the invention provides a composition comprising an MMO isolated from a plant comprising and expressing a recombinant MMO biosynthetic pathway sufficient to produce the MMO.

In an aspect the invention provides a method of isolating an MMO comprising isolating the MMO from a plant comprising and expressing a recombinant MMO biosynthetic pathway sufficient to produce the MMO.

In embodiments the invention provides a plant, method or composition herein, wherein the plant is further comprising and expressing a eukaryotic (e.g. human) or prokaryotic (e.g. bacterial, such as E. coli) nucleotide sugar biosynthetic pathway sufficient to increase the production of the MMO.

In embodiments the invention provides a plant, method or composition herein, wherein the plant is further comprising and expressing a prokaryotic (e.g. bacterial, such as E. coli) nucleotide sugar biosynthetic pathway sufficient to increase the production of the MMO, wherein the use of a prokaryotic (as opposed to a eukaryotic) pathway substantially avoids pathway feedback inhibition in the plant.

In embodiments the invention provides a plant, method or composition herein, wherein the plant is further comprising and expressing a prokaryotic (e.g. bacterial, such as E. coli) nucleotide sugar biosynthetic pathway sufficient to increase the production of the MMO, wherein the pathway comprises one, two or three pathways (and corresponding enzymes):
a) GDP-fucose (ManA, ManB, ManC, Gmd, WcaG),
b) UDP-galactose (pgi, pgm, GalU, GalE), and
c) UDP-GlcNAc (glmS, glmM, glmU).

In embodiments the invention provides a plant, method or composition herein, wherein the MMO is of a mammal selected from human, cow and goat.

In embodiments the invention provides a plant, method or composition herein, wherein the MMO is a human milk oligosaccharide (HMO).

In embodiments the invention provides a plant, method or composition herein, the plant comprising and expressing a plurality of recombinant MMO biosynthetic pathways sufficient to produce a plurality (e.g. at least 2, 3, 4, 5, 6, 7, 8, 9 or 10) of different MMOs.

In embodiments the invention provides a plant, method or composition herein, wherein the plant is a dicot or monocot plant, such as tobacco, potato, tomato, sorghum, soybean, sugarcane, Arabidopsis, rice, sugar beet, wheat, cassava, and oat.

In embodiments the invention provides a plant, method or composition herein, further comprising isolating the resultant MMO from the plant.

In aspects and embodiments the invention provides:

a composition, plant or plant part herein, producing a mammalian milk oligosaccharide (MMO), comprising and expressing a recombinant MMO biosynthetic pathway sufficient to produce the MMO, wherein the pathway comprises recombinant enzymes:
a β1-4 galactosyltransferase (β1-4-GalT; e.g. GalTPM1141), which glycosylates glucose using UDP-galactose to make lactose; and
a β1-3 N-acetylglucosaminyltransferase (β1-3-GnT; e.g. NmLgtA) generates lacto-N-triose (LNTII) through the transfer of GlcNAC from UDP-GlcNAc to the galactose in the lactose; and at least one of:
a β1-3-galactosyltransferase (β1-3-GalT; e.g. Cvβ3GalT) and a β1-4-GalT (e.g. HP0826 or NmLgtB), convert the LNTII to lacto-N-tetraose (LNT) and lacto-N-neotetraose (LNnT), respectively.

In aspects and embodiments the invention provides:
a composition, plant or plant part herein, comprising and expressing of a β1-4-GalT (e.g. GalTPM1141), a β1-3-GlcNAcT (e.g. NmLgtA), a β1-4-GalT (e.g. HP0826 or NmLgtB), and a β1-3-GalT (e.g. Cvβ3GalT) (FIG. 1) to produce LNnT and LNT;
a composition, plant or plant part herein producing non-fucosylated neutral MMOs (nMMOs, preferably nHMOs) comprising a lactose core with decorations of galactose and N-acetylglucosamine (GlcNAc);
a composition, plant or plant part herein producing fucosylated MMOs (preferably HMOs), particularly comprising a lactose core decorated with one or more fucose moieties;
a composition, plant or plant part herein comprising and expressing a β1-4-GalT (e.g. GalTPM1141), a β1-3-GlcNAcT (e.g. NmLgtA), a β1-4-GalT (e.g. HP0826 or NmLgtB), a β1-3-GalT (e.g. Cvβ3GalT) and a α1-2-fucosyltransferase (α1-2-FucT) (e.g. Te2FT) (FIG. 1) to produce lacto-N-fucopentaose I (LNFPI), a fucosylated pentasaccharide generated by the addition of an α-1,2-linked fucose to the terminal galactose in lacto-N-tetraose (LNT);
a composition, plant or plant part herein, producing fucosyllactose, lacto-N-tetraose or lacto-N-neotetraose, and lacto-N-fucopentaose I (and particularly, 2′FL, LNT, LNnT and LNFPI) respectively (FIG. 3, Table 1);
a composition, plant or plant part herein, wherein to improve LNFPI yields, nucleotide sugar biosynthetic pathways (FIG. 1B) are expressed to increase the availability of GDP-fucose (ManA, ManB, ManC, Gmd, WcaG), UDP-galactose (pgi, pgm, GalU, GalE), and UDP-GlcNAc (glmS, glmM, glmU) combinatorially (FIG. 4);
a composition, plant or plant part herein, wherein when expressing the LNFP1 biosynthetic pathway (FIG. 1A) and the GDP-fucose biosynthetic pathway (FIG. 1B), there the plant metabolism harbors the innate ability to further glycosylate simple MMOs to create MMOs of higher complexity (such as lactodifucotetraose (LDFT) and lacto-N-difucohexaose I (LNDFHI); FIG. 3, Table 1). that have not been previously produced through any other heterologous hosts;
a composition, plant or plant part herein, producing acidic MMOs, particularly comprising a lactose or nMMO core with one or more N-acetylneuraminic acid (Neu5Ac) moieties, comprising in the plant
a mammalian CMP-Neu5Ac pathway, a UDP-GlcNAc pathway and aMMO biosynthetic genes resulting in production of aMMOs, particularly production of CMP-Neu5Ac;
a plant herein, comprising and expressing a three gene bacterial pathway (neuA, neuB, neuC) or a four gene mammalian pathway (GNE, NANS, NMANP, CMAS) which utilize UDP-GlcNAc as a precursor for the production of CMP-Neu5Ac;
a composition, plant or plant part herein, comprising and expressing a mammalian CMP-Neu5 Ac pathway, GalTPM1141, NmLgtA, Cvβ3GalT, HpO826 (or NmnLgtB) and either PmSt3 or St6 (FIG. 1A) to produce 3′SL, 6′SL and isomers of LST;
a composition, plant or plant part herein, that is a stable, transgenic plant, comprising constructs for the constitutive production of 2′FL and LNFPI (e.g. in stably transformed N. benthamiana); in embodiments, MMO10 (FIG. 6) contains genes for the four enzymes required to produce lactose, 2′FL, LNTII, LNT, and LNFPI connected via 2A peptides to allow multiple coding sequences to be driven by a single promoter, wherein each transcriptional unit is driven by a strong constitutive promoter to enable MMO production in all tissues, or MMO11 (FIG. 6), which contains a GDP-D-mannose-4,6-dehydratase (Gmd) from the GDP-fucose pathway, wherein both constructs provide the production of multiple MMOs, specifically 2′FL (FIG. 7) and LNFPI (FIG. 8);
a method of producing a mammalian milk oligosaccharide (MMO) in a plant herein, comprising growing the plant comprising and expressing a recombinant MMO biosynthetic pathway sufficient to produce the MMO;
a composition comprising an MMO isolated from a plant herein comprising and expressing a recombinant MMO biosynthetic pathway sufficient to produce the MMO;
a food product composition comprising composition, plant or plant part (such as leaves, stems, roots, seeds, fruit or flowers) herein comprising and expressing a recombinant MMO biosynthetic pathway sufficient to produce the MMO.

The invention encompasses all combinations of the particular embodiments recited herein, as if each combination had been laboriously recited.

### BRIEF DESCRIPTION OF THE DRAWINGS

FIGS. 1A-B. A) MMO biosynthetic pathways used in this study. B) Nucleotide sugar biosynthetic pathways.

FIG. 2. Production of neutral MMOs in planta. Extracted mass chromatogram identifying the in planta production of Lacto-N-tetraose/Lacto-N-neotetraose (LNT/LNnT), 4_1_0_0, 5_2_0_0 and 6_1_0_0. Data collected using liquid chromatography-MS with a HILIC column. Numbered codes refer to the number of Hexose_HexNAc_Deoxyhexose_N-acetylneuraminic acid saccharides in the oligosaccharide as determined by MS/MS fragmentation.

FIG. 3. Production of fucosylated MMOs in planta. Extracted ion chromatogram identifying the in planta production of 2′-fucosyllactose (2′FL), lacto-N-fucopentaose I (LNFPI), lactodifucotetraose (LDFT), lacto-N-difucohexaose I (LNDFHI), and 4_1_1_0. Data collected using liquid chromatography-mass spectrometry with a HILIC column. Numbered codes refer to the number of Hexose_HexNAc_Deoxyhexose_N-acetylneuraminic acid saccharides in the oligosaccharide as determined by MS/MS fragmentation.

FIGS. 4A-B. Optimization of fucosylated MMO production. A) Quantification of LNFPI produced when expressing the LNFPI and nucleotide sugar biosynthetic pathways. B) Oligosaccharide profiles of leaf tissue expressing various nucleotide sugar biosynthetic pathways. Negative control produces negligible amounts of oligosaccharides compared to experimental samples. Hexose, HexNAc and deoxyhexose refer to the presence of at least one hexose sugar, N-acetylhexosamine sugar, and deoxyhexose sugar, respectively.

FIG. 5. Production of neutral MMOs in planta. Extracted ion chromatogram identifying the in planta production of 3′-sialyllactose (3′SL), 6′-sialyllactose (6′SL), sialyllacto-N-neotetraose d (LSTd), sialyllacto-N-neotetraose c (LSTc), and 4_1_0_1. Data was collected using liquid chromatography-mass spectrometry with a HILIC column. Numbered codes refer to the number of Hexose_HexNAc_Deoxyhexose_N-acetylneuraminic acid saccharides in the oligosaccharide as determined by MS/MS fragmentation.

FIG. 6. Constructs used in the generation of stably transformed N. benthamiana.

FIG. 7. Identification of 2′-fucosyllactose (2′FL) produced in stably transform N. benthamiana. Data was collected using liquid chromatography-mass spectrometry with a porous graphitic carbon column.

FIG. 8. Identification of lacto-N-fucopentaose I (LNFPI) produced in stably transform N. benthamiana. Data was collected using liquid chromatography-mass spectrometry with a porous graphitic carbon column.

### DESCRIPTION OF PARTICULAR EMBODIMENTS OF THE INVENTION

### Plant Material

Nicotiana benthamiana was grown in 3.5 inch square pots in a controlled environment facility under a 12/12 day/night cycle (12 hours light, 12 hours dark) at ˜100 μmol photons m⁻² sec⁻¹. Daytime temperatures were 26° C., and night temperatures were 25° C. Relative humidity was between 60-75%. Plants used in this study were 4 weeks old.

### Cloning

Putative and known human milk oligosaccharide biosynthetic genes were either synthesized or acquired from other labs. Genes were PCR amplified. Amplified candidate genes were then cloned into the binary vector PMS057 using Golden Gate assembly, Gibson assembly, or standard digestion and ligation assembly. 2-4 μL of the assembly reactions were transformed into XL1B chemically competent E. coli cells via heat shock as previously described. Colonies were selected on LB agar plates containing 50 μg/mL kanamycin and sequence-verified using Sanger sequencing (McLab). Sequence verified plasmids were used to transform Agrobacterium tumefaciens str. GV3101 by electroporation as described by. Competent cells were then plated on LB agar plates containing 50 μg/mL rifampicin, 10 μg/mL gentamicin, 50 μg/mL kanamycin.

Transient Expression in N. benthamiana

Overnight cultures of A. tumefaciens str. GV3101 were grown in LB to an OD600 between 0.8 and 1.2. Cultures were centrifuged at 4000×G for 10 min and the supernatant was removed. Bacterial pellets were resuspended in infiltration media (10 mM MgCl₂, 10 mM MES, 500 μM acetosyringone, pH: 5.6). Following an hour incubation, Agrobacterium strains containing HMO biosynthetic genes were mixed in various combinations to a final OD600 of 0.5. A. tumefaciens strains were normalized to the level of the highest number of strains used in an experiment. For experiments that had less than the highest number of strains, an additional A. tumefaciens strain harboring the unrelated gene (dsRed) was added to reach a final OD600 of 0.5. An A. tumefaciens strain harboring the p19 silencing suppressor was used in all experiments at the same concentration as other strains. A. tumefaciens suspensions were syringe infiltrated into the abaxial side of the seventh leaf of 4-week old N. benthamiana.

### Extractions

N. benthamiana leaves were harvested 5 days post-infiltration. Major veins of N. benthamiana were removed from the leaf tissue, and the tissue was frozen in liquid nitrogen before lyophilization. Lyophilized leaf tissue was bead beaten using a single steel bead at 20 Hz for 10 min.

Oligosaccharides were extracted from lyophilized leaf tissue by ethanol precipitation. To each sample, 1 mL of 80% ethanol was added before homogenization on a bead mill at 4 m/s for 1 min. Samples were then precipitated overnight at −20° C. and centrifuged at 10,000 rpm for 15 min. The supernatant was transferred to a 2 mL screw-cap tube. The pellet was washed twice by adding 500 μL of 80% ethanol, homogenizing via bead mill for 1 min, and centrifuging at 10,000 rpm for 15 min. The supernatant and washes were combined and dried in a vacuum centrifuge. Dried supernatants were reconstituted in 200 μL of water and subjected to both C18 and porous graphitized carbon (PGC) solid phase extractions (SPE) in 96-well plate format. C18 cartridges containing 25 mg of stationary phase were first conditioned by two additions of 250 μL of acetonitrile (ACN) followed by four additions of 250 μL water. Samples were then loaded and eluted with two volumes of 200 μL water. PGC cartridges containing 40 mg of stationary phase were conditioned by addition of 400 μL water, 400 μL 80% (v/v) ACN and water, followed by two volumes of 400 μL water. The sample eluate from C18 SPE was then loaded, washed thrice with 500 μL water, and eluted using two volumes of 200 μL 40% (v/v) ACN and water. The purified extracts were dried in a vacuum centrifuge and reconstituted in 100 μL of water before injecting 5 μL for liquid chromatography-mass spectrometry (LC-MS) analysis.

Initial chromatographic separation was carried out using a Thermo Scientific Vanquish UHPLC system equipped with a Waters BEH C18 Amide column (HILIC) (1.7 μm, 100 mm×2.1 mm). A 10 min binary gradient was employed based on Xu et al. (2017): 0.0-4.0 min: 25-35% A; 4.0-8.50 min, 35-65% A; 8.50-8.70 min: 25% A. Mobile phase A consisted of 3% ACN (v/v) in water with 0.1% formic acid, and mobile phase B consisted of 95% ACN (v/v) in water with 0.1% formic acid.

Chromatographic separation of oligosaccharides was carried out on an Agilent 1260 Infinity II LC equipped with a Hypercarb PGC column from Thermo Scientific (5 μm, 150 mm×1 mm). A 45 min binary gradient was employed: 0.00-2.50 min: 0.0-0.0% A; 2.50-15.00 min: 0.0-16.0% B; 15.00-20.00 min: 16.0-58.0% B; 20.00-22.00 min: 58.0-100.0% B; 22.00-28.00 min: 100.0-100.0% B; 28.00-30.00 min: 100.0-0.0% B; 30.00-40.00 min: 0.0-0.0% B. Solvent A consisted of 3% (v/v) ACN in water+0.1% formic acid and solvent B consisted of 90% (v/v) ACN in water+0.1% formic acid.

For identification of HMOs, a Thermo Scientific qExactive mass spectrometer equipped with an electrospray ionization source was operated in positive ionization mode with the following parameters: scan range=133.4-2000 m/z, spray voltage=2.5 |kV|, capillary temperature=320° C., aux gas heater temperature=325° C., sheath gas flow rate=25, aux gas flow rate=8, sweep gas flow rate=3. MS/MS analysis was performed using stepped collision energies of 20, 30, 40 [eV].

For quantification of LNFPI and HMO profiling, mass spectral analysis was carried out on an Agilent 6530 Accurate-Mass Q-TOF MS operated in positive mode using data dependent acquisition. The gas temperatures were held at 150° C. The fragmentor, skimmer, octopole, and capillary were operated at 70 V, 55 V, 750 V, and 1800 V, respectively. The collision energy was based on the empirically derived linear formula (1.8×m/z−3.6). The reference mass used for calibration was 922.009798 m/z. The Agilent MassHunter Qualitative software was used for data analysis. Oligosaccharides were identified using an in-house library, their MS/MS spectra, and comparison to either authenticated standards or a pool of human milk oligosaccharides of known composition. The reference mass used for calibration was 922.009798 m/z.

### Results

### Production of Neutral HMOs

Non-fucosylated neutral HMOs (nHMOs) are composed of a lactose core with various decorations of galactose and N-acetylglucosamine (GlcNAc). nHMOs are the most abundant class of HMOs in breast milk, comprising 42-55% of HMOs (Totten et al., 2012). While nHMOs provide various health benefits, they also serve as the core scaffold for the production of various complex HMOs.

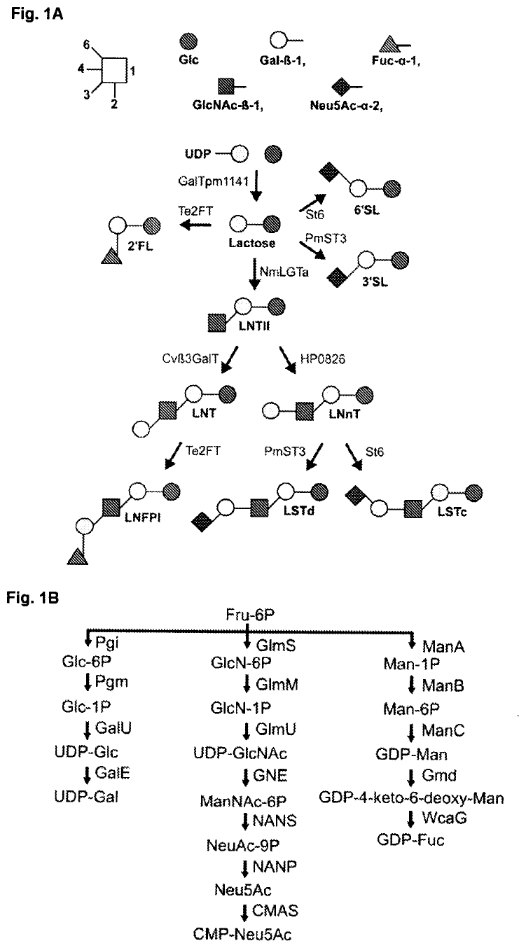

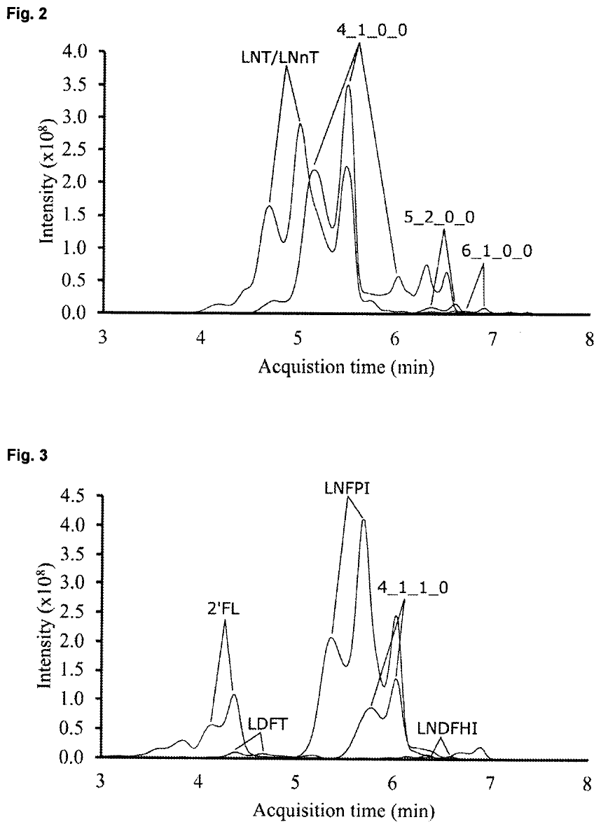

To produce nHMOs, lactose is first made by a β-1,4-galactosyltransferase, GalTPM1141, which glycosylates glucose using UDP-galactose (Parschat et al., 2020). Following lactose production, a β-1,3-N-acetylglucosaminyltransferase, NmLgtA, generates lacto-N-triose (LNTII) through the transfer of GlcNAC from UDP-GlcNAc to the galactose in lactose (McArthur et al., 2019). LNTII serves as a branch point for the production of various complex HMOs that can be further modified to generate large, complex HMOs. LNTII can then be used to generate lacto-N-tetraose (LNT) and lacto-N-neotetraose (LNnT), using β-1,3-galactosyltranserase, Cvβ3GalT, and a β-1,4-galactosyltransferase (e.g. HP0826 or NmLgtB), respectively (Fang et al., 2018; McArthur et al., 2019). Expression of GalTPM1141, NmLgtA, HP0826 (or NmLgtB), and Cvβ3GalT (FIG. 1) results in the production of ions with a m/z of 708.2565 and 708.2568 corresponding to LNnT and LNT respectively (FIG. 2, Table 1). Analysis using HILIC and PGC chromatography with analytical standards, retention time and MS/MS fragmentation further validated the identification of these structures, demonstrating the ability of plants to make nHMOs. Additionally, neutral HMOs with a higher degree of polymerization composed of 4 hexose sugars/1 hexNAc sugar, 5 hexose sugars/2 hexNAc sugars, and 6 hexose sugars/1 hexNAc were identified (FIG. 2, Table 1). These ions had values for m/z of 870.3091, 1235.4430, and 1194.4120, respectively. This displays the ability of plants to create HMOs with a high degree of polymerization.

### Production and Optimization of Fucosylated HMOs

Fucosylated HMOs (fHMOs) are a key component of breast milk and are characterized by the presence of a lactose or nHMO core decorated with one or more fucose moieties. While their abundance is dependent on the genotype of the mother, they are estimated to account for 35-50% of HMOs found in breast milk (Totten et al., 2012). fHMOs are of particular interest as they have distinct bioactivities from other HMOs. 2′-fucosyllactose (2′FL) is one of the most abundant HMOs found in human breast milk. Microbial production is currently able to produce 2′FL at a commercial scale; however, production of larger, abundant fHMOs such as lactodifucotetraose (LDFT), lacto-N-difucohexaose (LNDFH) and lacto-N-fucopentaose (LNFP) has not been possible at a commercial scale.

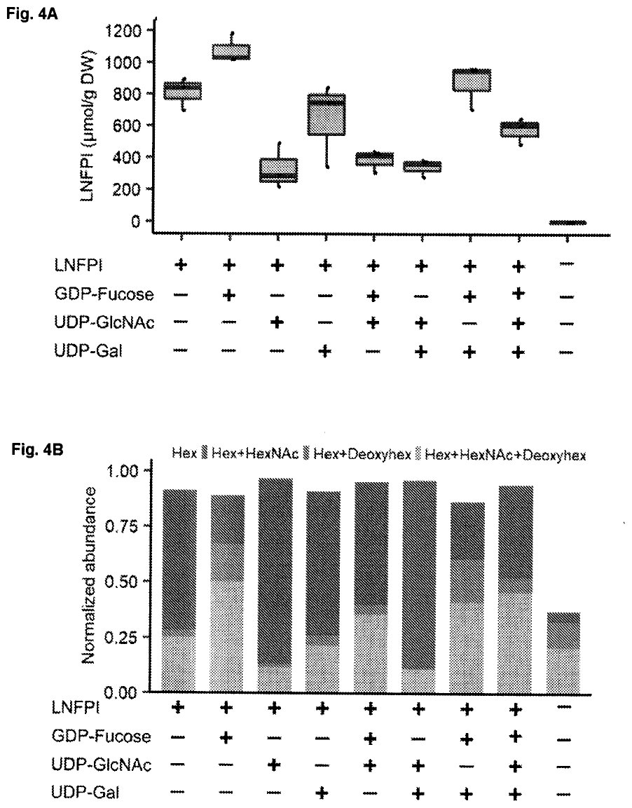

Lacto-N-fucopentaose I (LNFPI) is a fucosylated pentasaccharide generated by the addition of an α-1,2-linked fucose to the terminal galactose in lacto-N-tetraose (LNT). To produce LNFPI, we transiently expressed GalTPM1141, NmLgtA, Cvβ3GalT, HP0826 and Te2FT in N. benthamiana (FIG. 1). The expression of this pathway alone resulted in the production of an ions with m/z of 489.1824, 708.2568, 708.2565 and 854.3150, corresponding to fucosyllactose, lacto-N-tetraose or lacto-N-neotetraose, and lacto-N-fucopentaose I, respectively (FIG. 3, Table 1). Analysis using HILIC and PGC chromatography with analytical standards, retention time and MS/MS fragmentation identified these structures as 2′FL, LNT, LNnT and LNFPI (Table 1). Leaves expressing the LNFPI pathway produced LNFPI at a concentration of 809 μg/g DW (FIG. 4A). To improve LNFPI yields, nucleotide sugar biosynthetic pathways (FIG. 1B) were expressed to increase the availability of GDP-fucose (ManA, ManB, ManC, Gmd, WcaG), UDP-galactose (pgi, pgm, GalU, GalE), and UDP-GlcNAc (glmS, glmM, glmU) combinatorially (FIG. 4). The highest LNFPI yields were obtained by the expression of the GDP-fucose pathway with the LNFPI pathway, reaching a concentration of 1075 μg/g DW (1.33-fold improvement) (FIG. 4A). The increased yield following the expression of the GDP-fucose pathway suggests that native GDP-fucose levels are limiting (FIG. 4A); thus, our pathway engineering efforts demonstrate a metabolic strategy to address this bottleneck and improve overall yields of fHMOs like LNFPI.

HMO composition was also affected by changes in nucleotide sugar levels. Expression of the GDP-fucose pathway dramatically increased the relative amounts of oligosaccharides containing a deoxyhexose moiety, which likely corresponds to an increased abundance of fucosylated oligosaccharides (FIG. 4B). Expression of the UDP-GlcNAc pathway increased the relative amounts of oligosaccharides containing at least one hexose and one N-acetylhexosamine (hexNAc) (FIG. 4B). Together, these results display our ability to generate neutral and fucosylated HMOs in a single leaf and alter HMO composition by overexpression of various nucleotide sugar biosynthetic pathways.

In addition to producing the expected HMOs when expressing the LNFP1 biosynthetic pathway (FIG. 1A) and the GDP-fucose biosynthetic pathway (FIG. 1B), we identified the unanticipated production of two additional ions of m/z 635.2411 and 1000.3700. Analysis using HILIC and PGC chromatography with analytical standards, retention time and MS/MS fragmentation, the ion with m/z 635.2411 was identified as the HMO, lactodifucotetraose (LDFT), and the ion with m/z 1000.3700 was identified as the HMO, lacto-N-difucohexaose I (LNDFHI) (FIG. 3, Table 1). Since none of the enzymes used in this pathway are reported to have either α-1,3- or α-1,4-fucosyltransferase activity, we suspect that these additional glycosylations are products of endogenous plant enzymes; thus demonstrating some of the natural unanticipated advantages of using plants as a chassis for the production of HMOs. The discovery that plants have enzymes to fill in the last steps involved in making HMOs such as LDFT and LNDFHI highlight how plants may serve as a more attractive host for production of HMOs than current microbial platforms. Specifically, plant metabolism harbors the innate ability to further glycosylate simple HMOs to create HMOs of higher complexity that have not been previously produced through any other heterologous hosts.

### Production of Acidic HMOs

Acidic HMOs (aHMOs) are a class of HMO characterized by the presence of a lactose or nHMO core with one or more N-acetylneuraminic acid (Neu5Ac) moieties. aHMOs represent a structurally diverse class of HMOs, accounting for 12-14% of HMOs found in human breast milk (Totten et al., 2012). While aHMOs are not currently commercially available, simple aHMOs, such as 3′-sialyllactose (3′SL) and 6′-sialyllactose (6′SL), have been produced in E. coli (Ruffing & Chen, 2006); however, microbial systems have been unable to produce larger aHMOs, such as isomers of sialyllacto-N-tetraose (LST), To overcome this limitation, we sought to produce both simple and complex aHMOs using transient expression in N. benthamiana.

Since plants do not natively produce CMP-Neu5Ac, we tested a three gene bacterial pathway (neuA, neuB, neuC) and a four gene mammalian pathway (GANE, NANS, NANP, CMAS) which utilize UDP-GlcNAc as a precursor for the production of CMP-Neu5Ac (Castilho et al., 2008; Vimr et al., 2004). Expression of the bacterial CMP-Neu5Ac pathway and aHMO biosynthetic genes did not produce any aHMOs. Expression of the mammalian CMP-Neu5Ac pathway, UDP-GlcNAc pathway and aHMO biosynthetic genes resulted in the successful production of aHMOs, displaying the successful production of CMP-Neu5Ac.

Simple aHMOs, such as 3′SL and 6′SL, are produced through the a2-3 or a2-6 sialylation of lactose. LST is a complex pentasaccharide aHMO with multiple isomers. Production of LST requires the production of Lacto-N-neotetraose (LNnT) and its subsequent addition of an α-2,6-linked or α-2,3-linked Neu5Ac. To produce 3′SL, 6′SL and isomers of LST, we expressed a mammalian CMP-Neu5Ac pathway, GalTPM1141, NmLgtA, Cvβ3GalT, Hp0826 (or NmLgtB) and either PmrS3 or St6 (FIG. 1A). Following HILIC LC-MS analysis a peak was identified for m/z 634,2189, indicating sialyllactose was present (FIG. 5). Further analysis using PGC chromatography with analytical standards, retention time and MS/MS fragmentation indicates the presence of two sialyllactose isomers, corresponding to 3′SL and 6′SL (FIG. 5, Table 1). Multiple peaks are also present for m/z 999.3483, indicating the production of LST. Further analysis using PGC chromatography with analytical standards, retention time, and MS/MS fragmentation indicate the production of LSTc and LSTd in planta (FIG. 5, Table 1). This is especially notable, as this is the first successful production of LST in a heterologous host.

The production of 3′SL, 6′SL, LNnT, LNT, and multiple isomers of LST in a heterologous host has never been described before. Additionally, this is the first demonstration of producing LST isomers in a heterologous host, displaying the utility of plants as a platform. The production of aHMOs will serve as a starting point for the production of HMOs decorated with both fucose and Neu5Ac moieties.

### Production of HMOs in Stably Transformed Plants

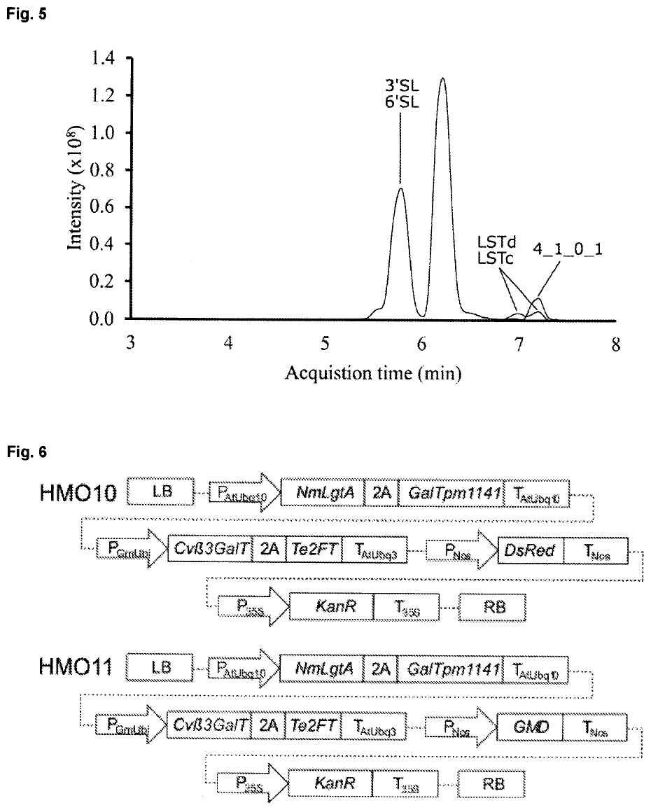

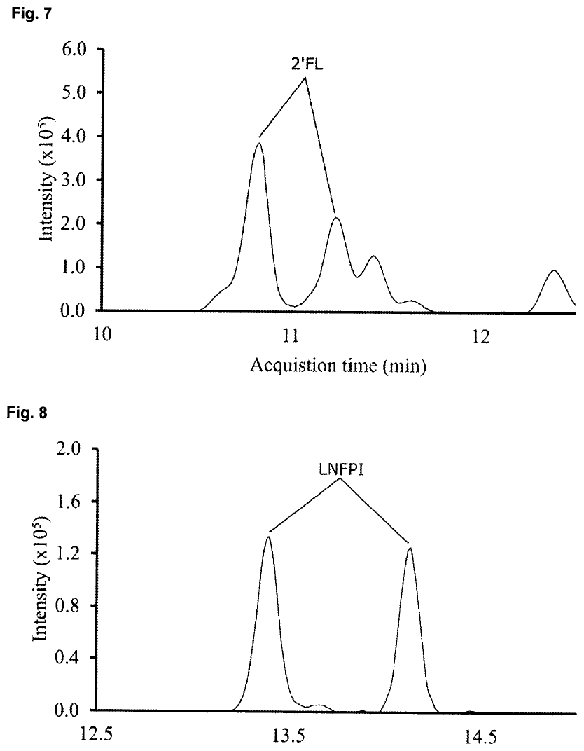

To test the ability of stable transgenic plants to produce HMOs, we generated two constructs for the constitutive production of 2′FL and LNFPI in stably transformed N. benthamiana. HMO10 (FIG. 6) contains genes for the four enzymes required to produce lactose, 2′FL, LNTII, LNT, and LNFPI connected via 2A peptides to allow multiple coding sequences to be driven by a single promoter. Each transcriptional unit is driven by a strong constitutive promoter to enable HMO production in all tissues. To explore the effects of overexpressing portions of the GDP-fucose pathway, we also generated stable lines expressing HMO11 (FIG. 6), which contains a GDP-D-mannose-4,6-dehydratase (Gmd) from the GDP-fucose pathway. Both constructs enabled the production of multiple HMOs, specifically 2′FL (FIG. 7) and LNFPI (FIG. 8).

### REFERENCES

Castilho, A., et al. (2008). Construction of a Functional CMP-Sialic Acid Biosynthesis Pathway in Arabidopsis. Plant Physiology, 147(1), 331-339.
Fang, J.-L., Tsai, T.-W., Liang, C.-Y., Li, J.-Y., & Yu, C.-C. (2018). Enzymatic Synthesis of Human Milk Fucosides α1,2-Fucosyl para-Lacto-N-Hexaose and its Isomeric Derivatives. Advanced Synthesis & Catalysis, 360(17), 3213-3219. https://doi.org/10.1002/adsc.201800518
McArthur, J. B., Yu, H., & Chen, X. (2019). A Bacterial β1-3-Galactosyltransferase Enables Multigram-Scale Synthesis of Human Milk Lacto-N-tetraose (LNT) and Its Fucosides. ACS Catalysis, 9(12), 10721-10726. https://doi.org/10.1021/acscatal.9b03990
Parschat, K., et al. (2020). High-Titer De Novo Biosynthesis of the Predominant Human Milk Oligosaccharide 2′-Fucosyllactose from Sucrose in Escherichia coli. ACS Synthetic Biology, 9(10), 2784-2796. https://doi.org/10.1021/acssynbio.0c00304
Ruffing, A., & Chen, R. R. (2006). Metabolic engineering of microbes for oligosaccharide and polysaccharide synthesis. Microbial Cell Factories, 5(1), 25. https://doi.org/10.1186/1475-2859-5-25
Totten, S. M., et al. (2012). Comprehensive Profiles of Human Milk Oligosaccharides Yield Highly Sensitive and Specific Markers for Determining Secretor Status in Lactating Mothers. Journal of Proteome Research, 11(12), 6124-6133. https://doi.org/10.1021/pr300769g
Virnr, E. R., et al. (2004). Diversity of Microbial Sialic Acid Metabolism. Microbiology and Molecular Biology Reviews. https://doi.org/10.1128/MMBR.68.1.132-153.2004
Xu, G, et al. (2017). Absolute quantitation of human milk oligosaccharides reveals phenotypic variations during lactation. The Journal of Nutrition, 147(1), 117-124.

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

| | | | | | | | Retention | MS/MS (top |
|---|---|---|---|---|---|---|---|---|
| Name | Hex | HexNAc | Deoxyhex | Neu5Ac | Enzymes | m/z (exptl) | time (min) | 3) |
| 2′FL* | 2 | 0 | 1 | 0 | GalTpm1141, NmLgtA, Cvβ3GalT, | 489.1824 | 10.97 | N/A |
| | | | | | Te2FT, ManA, ManC, Gmd, WcaG | | | |
| 3′SL* | 2 | 0 | 0 | 1 | GalTpm1141, NmLgtA, Hp0826, | 634.2183, 634.2189 | 17.18, 17.85 | 163.0604, |
| | | | | | PmSt3, GNE, NANS, NANP, CMAS | | | 274.0928, |
| | | | | | | | | 292.1034 |
| 6′SL* | 2 | 0 | 0 | 1 | GalTpm1141, NmLgtA, Cvβ3Gal, | 634.2197, 634.2002 | 13.40, 14.61 | N/A |
| | | | | | Hp0826, St6, GNE, NANS, NANP, | | | |
| | | | | | CMAS | | | |
| LNT* | 3 | 1 | 0 | 0 | GalTpm 1141, NmLgtA, Cvβ3GalT | 708.2568, 708.2571 | 14.33, 13.95 | 186.0765, |
| | | | | | | | | 204.0872, |
| | | | | | | | | 366.1404 |
| LNnT* | 3 | 1 | 0 | 0 | GalTpm1141, NmLgtA, Hp0826 | 708.2565 | 14.29 | 138.0553, |
| | | | | | | | | 168.0659, |
| | | | | | | | | 366.1404 |
| LSTc* | 3 | 1 | 0 | 1 | GalTpm1141, NmLgtA, Cvβ3Gal, | 999.3483 | 18.21 | N/A |
| | | | | | Hp0826, St6, GNE, NANS, NANP, | | | |
| | | | | | CMAS | | | |
| LSTd* | 3 | 1 | 0 | 1 | GalTpm1141, NmLgtA, Hp0826, | 999.3484 | 19.37 | N/A |
| | | | | | PmSt3, GNE, NANS, NANP, CMAS | | | |
| LNFPI* | 3 | 1 | 1 | 0 | GalTpm1141, NmLgtA, Cvβ3GalT, | 854.3150, 854.3159, | 13.15, 13.91 | 204.0872, |
| | | | | | Te2FT, ManA, ManC, Gmd, WcaG | | | 366.1404, |
| | | | | | | | | 512.1996 |
| LDFT* | 2 | 0 | 2 | 0 | GalTpm 1141, NmLgtA, Cvβ3GalT, | 635.2411 | 10.73 | N/A |
| | | | | | Te2FT, ManA, ManC, Gmd, WcaG | | | |
| LNDFHI* | 4 | 1 | 2 | 0 | GalTpm1141, NmLgtA, Cvβ3GalT, | 1000.3700, 1000.3750 | 10.23, 10.62 | N/A |
| | | | | | Te2FT, ManA, ManC, Gmd, WcaG | | | |
| 4100 | 4 | 1 | 0 | 0 | GalTpm1141, NmLgtA, Cvβ3Gal, | 870.3091 | 17.40, 17.70 | 204.0872, |
| | | | | | Hp0826 | | | 366.1404, |
| | | | | | | | | 528.1935 |
| 5200 | 5 | 2 | 0 | 0 | GalTpm1141, NmLgtA, Cvβ3Gal, | 1235.443 | 18.15 | 366.1404, |
| | | | | | Hp0826 | | | 731.2735, |
| | | | | | | | | 1235.4497 |
| 6100 | 6 | 1 | 0 | 0 | GalTpm1141, NmLgtA, Cvβ3Gal, | 1194.4120, 1194.4130 | 18.51, 18.71 | 204.0872, |
| | | | | | Hp0826 | | | 366.1404, |
| | | | | | | | | 690.2472, |
| 4000 | 4 | 0 | 0 | 0 | GalTpm1141, NmLgtA, Cvβ3Gal, | 667.2294 | 17.5 | 163.0606, |
| | | | | | Hp0826 | | | 325.1144, |
| | | | | | | | | 630.7106 |
| 4101a | 4 | 1 | 0 | 1 | GalTpm1141, NmLgtA, Hp0826, | 1161.403 | 19.8 | 366.1406, |
| | | | | | PmSt3, GNE, NANS, NANP, CMAS | | | 292.1034, |
| | | | | | | | | 657.2365 |
| 4101b | 4 | 1 | 0 | 1 | GalTpm1141, NmLgtA, Hp0826, | 1161.402 | 20.61 | 366.1405, |
| | | | | | PmSt3, GNE, NANS, NANP, CMAS | | | 292.1036, |
| | | | | | | | | 657.2365 |
| 4101c | 4 | 1 | 0 | 1 | GalTpm1141, NmLgtA, Cvβ3GalT, | 1161.403 | 19.89 | 366.1406, |
| | | | | | PmSt3, GNE, NANS, NANP, CMAS | | | 292.1041, |
| | | | | | | | | 657.2370 |
| 4110a | 4 | 1 | 1 | 0 | GalTpm1141, NmLgtA, Cvβ3GalT, | 1016.365 | 14.66 | 204.0872, |
| | | | | | Te2FT, ManA, ManC, Gmd, WcaG | | | 366.1403, |
| | | | | | | | | 512.1988 |
| 4110b | 4 | 1 | 1 | 0 | GalTpm 1141, NmLgtA, Cvβ3GalT, | 1016.365 | 15 | 204.0874, |
| | | | | | Te2FT, ManA, ManC, Gmd, WcaG | | | 366.1396, |
| | | | | | | | | 512.1982 |
| 4110c | 4 | 1 | 1 | 0 | GalTpm1141, NmLgtA, Cvβ3GalT, | 1016.365 | 16.57 | 366.1404, |
| | | | | | Te2FT, ManA, ManC, Gmd, WcaG | | | 204.0872, |
| | | | | | | | | 512.1989 |

Table 1. Chromatographic retention time and mass spectrum peaks of HMOs identified using liquid chromatography-mass spectrometry with a porous graphitic carbon column. * denotes identification with an authenticated standard.

### HMO Gene Sequences: Gene Name, Accession, Nucleotide and Amino Acid Sequence

GalTpm1141 AEC04686
(SEQ ID NO: 1)
ATGCCCATGACGAACTACGTAATATCACTAAGTTCAGCCAGAGAGCGTAGGCGTCATGT
GATGAACGAATTTTCCAAGCACCACGTTCCATTCCAGTTCTTCGACGCAGTTTCACCATCTTCT
CAGCTGGATTTCCTCATTCAAAGACTTGTGCCTAACCTTAACGGAACAAGCCTTACAGGTGGAG
AGAAAGGGTGCCTTATCTCACATGTGGCCTTATGGCATAAGTGCATACAAGACAATCTGCCATA
TATAGCAATCTTCGAAGATGACATACTCCTTGGCAGGGACGCCCGTGCCTTCTTGGCAGAGGAC
GAGTGGCTATTCTCTCGATTCAACTGTGACGATATCTTCATTATCAGACTGGAAACTTTTCTTC
AGGAAACTATCTGCGAAGCGCTCCCCAACCCCGTGTCATATTGCGGCAGGGACTTCTTGGCACT
TAAAGATGAACACCTTGGGACGGCTGGTTATATCATTTCACTTGGGGCCGCCAAATATTTATTG
GAAATCTTTAAGAACATGGAGAGTAACAACATTTTCCCAATCGACCACTTGATTTTCAATCGTT
TCTTAGCGGGTGAAGAACTGATGGTTTACCAGCTATCTCCTGCTCTGTGCATTCAAGAGCTTCA
ATTGAACGAAAACGAGTCTTTGTTGGACAGCCAACTTGAGTCCGAACGCAAGAACTATCGTCTT
GCTGAGAAGGCTAGAAAGAAGAAAACCTGGCGTGAAAAGGTTTACCACATCTTCACAAAGCCAC
AAAGAATGCTTAAGAAAAGAAAGGAAAGGGCTGAGAAGAATGCCAAAATGAAACTCAAATGCAT
CGTCAAATTCGAGTAA
(SEQ ID NO: 2)
MPMTNYVISLSSARERRRHVMNEFSKHHVPFQFFDAVSPSSQLDFLIQRLVPNLNGTSL
TGGEKGCLISHVALWHKCIQDNLPYIAIFEDDILLGRDARAFLAEDEWLFSRENCDDIFIIRLE
TFLQETICEALPNPVSYCGRDFLALKDEHLGTAGYIISLGAAKYLLEIFKNMESNNIFPIDHLI
FNRFLAGEELMVYQLSPALCIQELQLNENESLLDSQLESERKNYRLAEKARKKKTWREKVYHIF
TKPQRMLKKRKERAEKNAKMKLKCIVKFE*
NmLgtA AAC44084.1
(SEQ ID NO: 3)
ATGCAGCCTTTAGTCAGCGTATTGATTTGCGCCTACAACGTAGAAAAATATTTCGCCCA
ATCATTAGCCGCCGTCGTGAATCAAACTTGGTGCAACTTGGATATTTTGATTGTCGATGACGGC
TCGACAGACGGTACGCTTGCCATTGCCAAGGATTTTCAAAAGCGGGACAGCCGTATCAAAATCC
TTGCACAAGCTCAAAATTCCGGCCTGATTCCCTCTTTAAACATCGGGCTGGACGAATTGGCAAA
GTCGGGGGGGGGGGAATATATTGCACGCACCGATGCCGACGATATTGCCGCCCCCGACTGGATT
GAGAAAATCGTGGGCGAGATGGAAAAAGACCGCAGCATCATCGCGATGGGCGCGTGGCTGGAAG
TGTTGTCGGAAGAAAAGGACGGCAACCGGCTGGCGCGGCATCACAGGCACGGCAAAATTTGGAA
AAAGCCGACCCGACACGAAGATATTGCCGACTTTTTCCCTTTCGGCAACCCCATACACAACAAC
ACGATGATTATGAGGCGCAGCGTCATTGACGGCGGTTTGCGTTACAACACCGAGCGGGATTGGG
CGGAAGATTACCAATTTTGGTACGATGTCAGCAAATTGGGCAGGCTGGCTTATTATCCCGAAGC
CTTGGTCAAATACCGCCTTCACGCCAATCAGGTTTCATCCAAATACAGCATCCGCCAACACGAA
ATCGCGCAAGGCATCCAAAAAACCGCCAGAAACGATTTTTTGCAGTCTATGGGTTTTAAAACCC
GGTTCGACAGCCTTGAATACCGCCAAATAAAAGCAGTAGCGTATGAATTGCTGGAGAAACATTT
GCCGGAAGAAGATTTTGAACGCGCCCGCCGGTTTTTGTACCAATGCTTCAAACGGACGGACACG
CCGCCCGCCGGCGCGTGGCTGGATTTTGCGGCAGACGGCAGGATGCGGCGGCTGTTTACCTTGA
GGCAATACTTCGGCATTTTGCACCGATTGCTGAAAAACCGTTAA
(SEQ ID NO: 4)
MQPLVSVLICAYNVEKYFAQSLAAVVNQTWCNLDILIVDDGSTDGTLAIAKDFQKRDSR
IKILAQAQNSGLIPSLNIGLDELAKSGGGEYIARTDADDIAAPDWIEKIVGEMEKDRSIIAMGA
WLEVLSEEKDGNRLARHHRHGKIWKKPTRHEDIADFFPFGNPIHNNTMIMRRSVIDGGLRYNTE
RDWAEDYQFWYDVSKLGRLAYYPEALVKYRLHANQVSSKYSIRQHEIAQGIQKTARNDFLQSMG
FKTRFDSLEYRQIKAVAYELLEKHLPEEDFERARRFLYQCFKRTDTPPAGAWLDFAADGRMRRL
FTLRQYFGILHRLLKNR
Cv?3GalT WP_048405302.1
(SEQ ID NO: 5)
ATGGACACCATCATGATTAAACGTCCGCTGGTTAGCGTTATTCTGCCGGTGAATAAAAA
CAATCCGCATCTGGAAGAAGCAATCCAGAGCATTAAAAACCAGACCTATAAAGAGCTGGAACTG
ATCATTATTGCCAACAACTGCGAGGATAACTTTTATAGCCTGCTGCTGAAATATCAGGACCAGA
AAACCAAAATTATCCGCACCAGCATCAAATATCTGCCGTTTAGCCTGAATCTGGGTGTTCATCT
GAGCCAGGGTGAATATATTGCACGTATGGATTCAGATGATATCAGCGTTCTGGATCGCATTGAA
AAACAGGTTAAACGCTTTCTGAATACACCGGAACTGAGCATTCTGGGTAGCAATGTTGAATATA
TCAATGAAGCCAGCGAAAGCATTGGCTATAGCAACTATCCGCTGGATCATAGCAGCATTGTTAA
TAGCTTTCCGTTTCGTTGTAATCTGGCACATCCGACCATTATGGTTAAAAAAGAAGTGATTACC
ACGCTTGGTGGCTATATGTATGGTAGCCTGAGCGAAGATTATGATCTGTGGATTCGTGCAAGCC
GTCATGGCAATTTCAAATTTAGCAATATTGATGAACCGCTGCTGAAGTACCGTATTCATAAAGG
TCAGGCAACCAATAAAAGCAACGCCTATAACATCTTTGCCTTTGATAGCAGCCTGAAAATCCGT
GAATTTCTGCTGAATGGTAATGTGCAGTATCTGCTGGGTGCAGCACGTGGTTTTTTTGCATTTC
TGTATGTGCGTTTCATCAAAAAATAA
(SEQ ID NO: 6)
MDTIMIKRPLVSVILPVNKNNPHLEEAIQSIKNQTYKELELIIIANNCEDNFYSLLLKY
QDQKTKIIRTSIKYLPFSLNLGVHLSQGEYIARMDSDDISVLDRIEKQVKRFLNTPELSILGSN
VEYINEASESIGYSNYPLDHSSIVNSFPFRCNLAHPTIMVKKEVITTLGGYMYGSLSEDYDLWI
RASRHGNFKFSNIDEPLLKYRIHKGQATNKSNAYNIFAFDSSLKIREFLLNGNVQYLLGAARGF
FAFLYVRFIKK*
Te2FT BAC08546.1
(SEQ ID NO: 7)
ATGATTATCGTTCACCTGTGCGGTGGCCTGGGCAACCAGATGTTCCAGTACGCAGCGGG
TCTGGCAGCAGCGCACCGTATCGGTTCTGAAGTGAAATTCGACACCCATTGGTTCGACGCGACT
TGTCTGCATCAGGGCCTGGAGCTGCGCCGTGTTTTTGGTCTGGAACTGCCGGAGCCGTCCTCTA
AAGACCTGCGTAAAGTTCTGGGCGCGTGTGTTCATCCGGCTGTTCGTCGTCTGCTGGCTGGCCA
CTTCCTGCATGGTCTGCGCCCGAAATCCCTGGTTATCCAGCCGCATTTCCACTACTGGACCGGT
TTTGAGCACCTGCCGGACAACGTTTACCTGGAGGGTTACTGGCAGTCCGAACGTTACTTCTCTA
ACATCGCGGATATCATCCGTCAGCAGTTTCGCTTCGTGGAACCGCTGGACCCGCACAACGCGGC
TCTGATGGATGAAATGCAGTCTGGTGTGTCTGTTTCTCTGCACATTCGTCGCGGCGATTACTTT
AACAACCCGCAGATGCGTCGCGTGCATGGCGTGGATCTGAGCGAATACTACCCGGCTGCTGTTG
CGACCATGATCGAGAAGACTAACGCAGAACGTTTCTACGTTTTCTCCGACGATCCGCAGTGGGT
TCTGGAGCATCTGAAGCTGCCGGTTAGCTATACCGTTGTGGATCACAACCGTGGTGCTGCGAGC
TACCGTGATATGCAGCTGATGTCCGCGTGCCGTCACCACATCATCGCGAACTCTACCTTCTCTT
GGTGGGGTGCGTGGCTGAACCCGCGTCCGGATAAAGTTGTTATCGCACCGCGCCACTGGTTTAA
CGTTGACGTTTTTGACACCCGCGATCTGTACTGCCCGGGTTGGATTGTTCTGTAA
(SEQ ID NO: 8)
MIIVHLCGGLGNQMFQYAAGLAAAHRIGSEVKFDTHWFDATCLHQGLELRRVFGLELPE
PSSKDLRKVLGACVHPAVRRLLAGHFLHGLRPKSLVIQPHFHYWTGFEHLPDNVYLEGYWQSER
YFSNIADIIRQQFRFVEPLDPHNAALMDEMQSGVSVSLHIRRGDYFNNPQMRRVHGVDLSEYYP
AAVATMIEKTNAERFYVESDDPQWVLEHLKLPVSYTVVDHNRGAASYRDMQLMSACRHHIIANS
TFSWWGAWLNPRPDKVVIAPRHWENVDVFDTRDLYCPGWIVL*
NmLgtB Q8KR92
(SEQ ID NO: 9)
ATGCAAAACCACGTTATCAGCTTAGCTTCCGCCGCAGAACGCAGGGCGCACATTGCCGA
TACCTTCGGCAGGCACGGCATCCCGTTTCAGTTTTTCGACGCACTGATGCCGTCTGAAAGGCTG
GAACAGGCAATGGCGGAACTCGTCCCCGGCTTGTCGGCGCACCCCTATTTGAGCGGAGTGGAAA
AAGCCTGCTTTATGAGCCACGCCGTATTGTGGAAGCAGGCATTGGACGAAGGTCTGCCGTATAT
CACCGTATTTGAGGACGACGTTTTACTCGGCGAAGGTGCGGAAAAATTCCTTGCCGAAGACGCT
TGGCTGCAAGAACGCTTTGACCCGGATACCGCCTTTATCGTCCGCTTGGAAACGATGTTTATGC
ACGTCCTGACCTCGCCCTCCGGCGTGGCGGATTACTGCGGGCGCGCCTTTCCGCTGTTGGAAAG
CGAACACTGGGGGACGGCGGGCTATATCATTTCCCGAAAAGCGATGCGGTTTTTCCTGGACAGG
TTTGCCGCCCTGCCGCCCGAAGGGCTGCACCCCGTCGATCTGATGATGTTCAGCGATTTTTTCG
ACAGGGAAGGAATGCCGGTTTGCCAGCTCAATCCCGCCTTGTGCGCCCAAGAGCTGCATTATGC
CAAGTTTCACGACCAAAACAGCGCATTGGGCAGCCTGATCGAACACGACCGCCTCCTGAACCGC
AAACAGCAAAGGCGCGATTCCCCCGCCAACACATTCAAACACCGCCTGATCCGCGCCTTGACCA
AAATCAGCAGGGAAAGGGAAAAACGCCGGCAAAGGCGCGAACAGTTCATTGTGCCTTTCCAATA
A
(SEQ ID NO: 10)
MQNHVISLASAAERRAHIADTFGRHGIPFQFFDALMPSERLEQAMAELVPGLSAHPYLS
GVEKACFMSHAVLWKQALDEGLPYITVFEDDVLLGEGAEKFLAEDAWLQERFDPDTAFIVRLET
MFMHVLTSPSGVADYCGRAFPLLESEHWGTAGYIISRKAMRFFLDRFAALPPEGLHPVDLMMFS
DFFDREGMPVCQLNPALCAQELHYAKFHDQNSALGSLIEHDRLLNRKQQRRDSPANTFKHRLIR
ALTKISREREKRRQRREQFIVPFQ*
HP0826 O25500
(SEQ ID NO: 11)
ATGCGTGTTTTTGCCATTTCTTTAAATCAAAAAGTGTGCGATACATTTGGTTTAGTTTT
TAGAGACACCACAACTTTACTCAATAGCATCAATGCCACCCACCACCAAGCGCAAATTTTTGAT
GCGATTTATTCTAAAACTTTTGAAGGCGGGTTGCACCCCTTAGTGAAAAAGCATTTACACCCTT
ATTTCATCACGCAAAACATCAAAGACATGGGGATTACAACCAATCTCATCAGTGAGGTTTCTAA
GTTTTATTACGCTTTAAAATACCATGCGAAGTTTATGAGCTTGGGGGAGCTTGGGTGCTATGCG
AGTCATTATTCCTTGTGGGAAAAATGCATAGAACTCAATGAAGCGATCTGTATTTTAGAAGACG
ATATAACCTTGAAAGAGGATTTTAAAGAGGGCTTGGATTTTTTAGAAAAACACATCCAAGAGTT
AGGCTATATCCGCTTGATGCATTTATTGTATGATGCCAGTGTAAAAAGTGAGCCATTGAGCCAT
AAAAACCACGAGATACAAGAGCGTGTGGGGATCATTAAAGCTTATAGCGAAGGGGTGGGGACTC
AAGGCTATGTGATCACGCCTAAGATTGCCAAAGTTTTTTTGAAATGCAGCCGAAAATGGGTTGT
TCCTGTGGATACGATAATGGACGCTACTTTTATCCATGGCGTGAAAAATCTGGTGTTACAACCT
TTTGTGATCGCTGATGATGAGCAAATCTCTACGATAGCACGAAAAGAAGAACCTTATAGCCCTA
AAATCGCCTTAATGAGAGAACTCCATTTTAAATATTTGAAATATTGGCAGTTTGTATAA
(SEQ ID NO: 12)
MRVFAISLNQKVCDTFGLVERDTTTLLNSINATHHQAQIFDAIYSKTFEGGLHPLVKKH
LHPYFITQNIKDMGITTNLISEVSKFYYALKYHAKFMSLGELGCYASHYSLWEKCIELNEAICI
LEDDITLKEDFKEGLDFLEKHIQELGYIRLMHLLYDASVKSEPLSHKNHEIQERVGIIKAYSEG
VGTQGYVITPKIAKVFLKCSRKWVVPVDTIMDATFIHGVKNLVLQPFVIADDEQISTIARKEEP
YSPKIALMRELHFKYLKYWQFV*
PMST3 AE004439
(SEQ ID NO: 13)
ATGGATAAATTTGCCGAACATGAAATTCCGAAAGCCGTTATTGTTGCAGGTAATGGTGA
AAGCCTGAGCCAGATTGATTATCGTCTGCTGCCGAAAAATTATGATGTGTTTCGCTGCAATCAG
TTTTATTTTGAAGAACGCTATTTTCTGGGCAATAAAATTAAAGCCGTGTTTTTTACACCGGGTG
TTTTTCTGGAACAGTATTATACCCTGTATCATCTGAAACGCAATAATGAATATTTTGTGGATAA
TGTGATTCTGAGCAGCTTTAATCATCCGACCGTTGATCTGGAAAAAAGCCAGAAAATTCAGGCC
CTGTTTATTGATGTGATTAATGGCTATGAAAAATATCTGAGCAAACTGACCGCCTTTGATGTTT
ATCTGCGCTATAAAGAACTGTATGAAAATCAGCGTATTACCAGCGGTGTTTATATGTGTGCAGT
TGCAATTGCAATGGGCTATACCGATATTTATCTGACCGGCATTGATTTTTATCAGGCCAGCGAA
GAAAATTATGCCTTTGATAATAAAAAACCGAATATTATTCGCCTGCTGCCGGATTTTCGCAAAG
AAAAAACCCTGTTTAGCTATCATAGCAAAGATATTGATCTGGAAGCCCTGAGCTTTCTGCAGCA
GCATTATCATGTGAATTTTTATAGCATTAGCCCGATGAGTCCGCTGAGCAAACATTTTCCGATT
CCGACCGTGGAAGATGATTGTGAAACCACCTTTGTTGCACCGCTGAAAGAAAATTATATTAATG
ATATTCTGCTGCCTCCGCATTTTGTGTATGAAAAACTGGGCGTCGACAAGCTTGCGGCCGCACT
CGAGTGA
(SEQ ID NO: 14)
MDKFAEHEIPKAVIVAGNGESLSQIDYRLLPKNYDVFRCNQFYFEERYFLGNKIKAVFF
TPGVFLEQYYTLYHLKRNNEYFVDNVILSSFNHPTVDLEKSQKIQALFIDVINGYEKYLSKLTA
FDVYLRYKELYENQRITSGVYMCAVAIAMGYTDIYLTGIDFYQASEENYAFDNKKPNIIRLLPD
FRKEKTLFSYHSKDIDLEALSFLQQHYHVNFYSISPMSPLSKHFPIPTVEDDCETTEVAPLKEN
YINDILLPPHFVYEKLGVDKLAAALE*
nst U60662.2
(SEQ ID NO: 15)
ATGGGCTTGAAAAAGGCTTGTTTGACCGTGTTGTGTTTGATTGTTTTTTGTTTCGGGAT
ATTTTATACATTTGACCGGGTAAATCAGGGGGAAAGGAATGCGGTTTCCCTGCTGAAGGAGAAA
CTTTTCAATGAAGAGGGGGAACCGGTCAATCTGATTTTCTGTTATACCATATTGCAGATGAAGG
TGGCGGAAAGGATTATGGCGCAGCATCCGGGCGAGCGGTTTTATGTGGTGCTGATGTCTGAAAA
CAGGAATGAAAAATACGATTATTATTTCAATCAGATAAAGGATAAGGCGGAGCGGGCGTACTTT
TTCCACCTGCCCTACGGTTTGAACAAATCGTTTAATTTCATTCCGACGATGGCGGAGCTGAAGG
TAAAGTCGATGCTGCTGCCGAAAGTCAAGCGGATTTATTTGGCAAGTTTGGAAAAAGTCAGCAT
TGCCGCCTTTTTGAGCACTTACCCGGATGCGGAAATCAAAACCTTTGACGACGGGACAGGCAAT
TTAATTCAAAGCAGCAGCTATTTGGGCGATGAGTTTTCTGTAAACGGGACGATCAAGCGGAATT
TTGCCCGGATGATGATCGGAGATTGGAGCATCGCCAAAACCCGCAATGCTTCCGACGAGCATTA
CACGATATTCAAGGGTTTGAAAAACATTATGGACGACGGCCGCCGCAAGATGACTTACCTGCCG
CTGTTCGATGCGTCCGAACTGAAGACGGGGGACGAAACGGGCGGCACGGTGCGGATACTTTTGG
GTTCGCCCGACAAAGAGATGAAGGAAATTTCGGAAAAGGCGGCAAAAAACTTCAAAATACAATA
TGTCGCGCCGCATCCCCGCCAAACCTACGGGCTTTCCGGCGTAACCACATTAAATTCGCCCTAT
GTCATCGAAGACTATATTTTGCGCGAGATTAAGAAAAACCCGCATACGAGGTATGAAATTTATA
CCTTTTTCAGCGGCGCGGCGTTGACGATGAAGGATTTTCCCAATGTGCACGTTTACGCATTGAA
ACCGGCTTCCCTTCCGGAAGATTATTGGCTCAAGCCGGTGTATGCCCTGTTTACCCAATCCGGC
ATCCCGATTTTGACATTTGACGATAAAAATTAA
(SEQ ID NO: 16)
MGLKKACLTVLCLIVFCFGIFYTFDRVNQGERNAVSLLKEKLFNEEGEPVNLIFCYTIL
QMKVAERIMAQHPGERFYVVLMSENRNEKYDYYFNQIKDKAERAYFFHLPYGLNKSFNFIPTMA
ELKVKSMLLPKVKRIYLASLEKVSIAAFLSTYPDAEIKTFDDGTGNLIQSSSYLGDEFSVNGTI
KRNFARMMIGDWSIAKTRNASDEHYTIFKGLKNIMDDGRRKMTYLPLFDASELKTGDETGGTVR
ILLGSPDKEMKEISEKAAKNFKIQYVAPHPRQTYGLSGVTTLNSPYVIEDYILREIKKNPHTRY
EIYTFFSGAALTMKDFPNVHVYALKPASLPEDYWLKPVYALFTQSGIPILTFDDKN*
st6 AB293985.1
(SEQ ID NO: 17)
ATGAAAAACTTTTTATTATTAACTTTAATATTACTTACTGCTTGTAATAATTCAGAAGA
AAATACACAATCTATTATTAAAAATGATATTAATAAAACTATTATTGATGAGGAGTATGTTAAT
TTAGAGCCAATTAATCAATCAAACATCTCTTTTACAAAACACTCTTGGGTACAAACTTGTGGTA
CGCAACAACTATTAACAGAACAAAATAAAGAGTCAATATCATTATCTGTAGTGGCGCCACGATT
AGATGACGATGAAAAGTACTGCTTTGATTTTAATGGTGTTAGTAATAAAGGTGAAAAATATATA
ACAAAAGTAACATTAAACGTAGTGGCTCCATCTTTAGAGGTTTATGTTGATCATGCATCTCTTC
CAACTCTTCAGCAGCTAATGGATATTATTAAATCGGAAGAAGAAAATCCTACAGCACAAAGATA
TATAGCTTGGGGGAGAATAGTTCCGACTGATGAGCAAATGAAAGAGTTAAATATTACATCGTTT
GCATTGATAAATAACCATACACCAGCTGACTTAGTACAAGAAATTGTTAAGCAAGCACAAACAA
AGCATAGATTGAATGTTAAACTTAGCTCTAACACTGCTCATTCATTTGATAATTTAGTGCCAAT
ACTAAAAGAATTAAATTCGTTTAATAACGTTACGGTAACAAATATAGATTTATATGATGATGGT
TCAGCAGAATATGTAAATTTATATAACTGGAGAGATACATTAAATAAAACAGATAATTTAAAAA
TTGGTAAAGATTATCTTGAGGATGTCATTAATGGTATCAATGAAGACACTTCAAATACAGGAAC
ATCATCTGTTTATAACTGGCAAAAACTATATCCAGCTAACTACCATTTTTTAAGAAAAGATTAT
TTAACTTTAGAACCATCATTACATGAGTTACGAGACTATATTGGTGATAGTTTAAAGCAAATGC
AATGGGATGGTTTCAAAAAATTCAATAGCAAACAACAAGAATTATTCTTATCGATTGTTAATTT
TGACAAACAAAAATTACAAAATGAATATAATTCATCTAATTTACCAAACTTTGTGTTTACAGGT
ACGACTGTATGGGCTGGTAACCATGAAAGAGAGTATTATGCGAAACAACAAATTAATGTCATTA
ATAATGCAATTAATGAATCGAGCCCACATTATTTAGGCAATAGTTATGATTTGTTCTTCAAAGG
TCACCCTGGTGGCGGTATCATTAATACATTAATAATGCAAAACTATCCTTCAATGGTTGATATT
CCATCAAAAATATCATTTGAAGTTTTGATGATGACAGATATGCTTCCTGATGCAGTTGCTGGTA
TAGCGAGCTCTTTATATTTCACGATACCAGCTGAAAAAATTAAATTTATAGTTTTTACATCGAC
AGAAACTATAACTGATCGTGAAACTGCTTTGAGAAGTCCTTTAGTTCAAGTAATGATAAAACTA
GGTATTGTAAAAGAAGAGAATGTACTTTTTTGGGCTGATCTGCCAAATTGTGAAACAGGTGTTT
GTATTGCAGTCTAG
(SEQ ID NO: 18)
MKNFLLLTLILLTACNNSEENTQSIIKNDINKTIIDEEYVNLEPINQSNISFTKHSWVQ
TCGTQQLLTEQNKESISLSVVAPRLDDDEKYCFDFNGVSNKGEKYITKVTLNVVAPSLEVYVDH
ASLPTLQQLMDIIKSEEENPTAQRYIAWGRIVPTDEQMKELNITSFALINNHTPADLVQEIVKQ
AQTKHRLNVKLSSNTAHSFDNLVPILKELNSFNNVTVTNIDLYDDGSAEYVNLYNWRDTLNKTD
NLKIGKDYLEDVINGINEDTSNTGTSSVYNWQKLYPANYHFLRKDYLTLEPSLHELRDYIGDSL
KQMQWDGFKKFNSKQQELFLSIVNFDKQKLQNEYNSSNLPNFVFTGTTVWAGNHEREYYAKQQI
NVINNAINESSPHYLGNSYDLFFKGHPGGGIINTLIMQNYPSMVDIPSKISFEVLMMTDMLPDA
VAGIASSLYFTIPAEKIKFIVFTSTETITDRETALRSPLVQVMIKLGIVKEENVLFWADLPNCE
TGVCIAV*
pgi P0A6T1
(SEQ ID NO: 19)
ATGAAAAACATCAATCCAACGCAGACCGCTGCCTGGCAGGCACTACAGAAACACTTCGA
TGAAATGAAAGACGTTACGATCGCCGATCTTTTTGCTAAAGACGGCGATCGTTTTTCTAAGTTC
TCCGCAACCTTCGACGATCAGATGCTGGTGGATTACTCCAAAAACCGCATCACTGAAGAGACGC
TGGCGAAATTACAGGATCTGGCGAAAGAGTGCGATCTGGCGGGCGCGATTAAGTCGATGTTCTC
TGGCGAGAAGATCAACCGCACTGAAAACCGCGCCGTGCTGCACGTAGCGCTGCGTAACCGTAGC
AATACCCCGATTTTGGTTGATGGCAAAGACGTAATGCCGGAAGTCAACGCGGTGCTGGAGAAGA
TGAAAACCTTCTCAGAAGCGATTATTTCCGGTGAGTGGAAAGGTTATACCGGCAAAGCAATCAC
TGACGTAGTGAACATCGGGATCGGCGGTTCTGACCTCGGCCCATACATGGTGACCGAAGCTCTG
CGTCCGTACAAAAACCACCTGAACATGCACTTTGTTTCTAACGTCGATGGGACTCACATCGCGG
AAGTGCTGAAAAAAGTAAACCCGGAAACCACGCTGTTCTTGGTAGCATCTAAAACCTTCACCAC
TCAGGAAACTATGACCAACGCCCATAGCGCGCGTGACTGGTTCCTGAAAGCGGCAGGTGATGAA
AAACACGTTGCAAAACACTTTGCGGCGCTTTCCACCAATGCCAAAGCCGTTGGCGAGTTTGGTA
TTGATACTGCCAACATGTTCGAGTTCTGGGACTGGGTTGGCGGCCGTTACTCTTTGTGGTCAGC
GATTGGCCTGTCGATTGTTCTCTCCATCGGCTTTGATAACTTCGTTGAACTGCTTTCCGGCGCA
CACGCGATGGACAAGCATTTCTCCACCACGCCTGCCGAGAAAAACCTGCCTGTACTGCTGGCGC
TGATTGGCATCTGGTACAACAATTTCTTTGGTGCGGAAACTGAAGCGATTCTGCCGTATGACCA
GTATATGCACCGTTTCGCGGCGTACTTCCAGCAGGGCAATATGGAGTCCAACGGTAAGTATGTT
GACCGTAACGGTAACGTTGTGGATTACCAGACTGGCCCGATTATCTGGGGTGAACCAGGCACTA
ACGGTCAGCACGCGTTCTACCAGCTGATCCACCAGGGAACCAAAATGGTACCGTGCGATTTCAT
CGCTCCGGCTATCACCCATAACCCGCTCTCTGATCATCACCAGAAACTGCTGTCTAACTTCTTC
GCCCAGACCGAAGCGCTGGCGTTTGGTAAATCCCGCGAAGTGGTTGAGCAGGAATATCGTGATC
AGGGTAAAGATCCGGCAACGCTTGACTACGTGGTGCCGTTCAAAGTATTCGAAGGTAACCGCCC
GACCAACTCCATCCTGCTGCGTGAAATCACTCCGTTCAGCCTGGGTGCGTTGATTGCGCTGTAT
GAGCACAAAATCTTTACTCAGGGCGTGATCCTGAACATCTTCACCTTCGACCAGTGGGGCGTGG
AACTGGGTAAACAGCTGGCGAACCGTATTCTGCCAGAGCTGAAAGATGATAAAGAAATCAGCAG
CCACGATAGCTCGACCAATGGTCTGATTAACCGCTATAAAGCGTGGCGCGGTTAA
(SEQ ID NO: 20)
MKNINPTQTAAWQALQKHFDEMKDVTIADLFAKDGDRFSKFSATFDDQMLVDYSKNRIT
EETLAKLQDLAKECDLAGAIKSMFSGEKINRTENRAVLHVALRNRSNTPILVDGKDVMPEVNAV
LEKMKTFSEAIISGEWKGYTGKAITDVVNIGIGGSDLGPYMVTEALRPYKNHLNMHFVSNVDGT
HIAEVLKKVNPETTLFLVASKIFTTQETMINAHSARDWFLKAAGDEKHVAKHFAALSTNAKAVG
EFGIDTANMFEFWDWVGGRYSLWSAIGLSIVLSIGFDNFVELLSGAHAMDKHFSTTPAEKNLPV
LLALIGIWYNNFFGAETEAILPYDQYMHRFAAYFQQGNMESNGKYVDRNGNVVDYQTGPIIWGE
PGTNGQHAFYQLIHQGTKMVPCDFIAPAITHNPLSDHHQKLLSNFFAQTEALAFGKSREVVEQE
YRDQGKDPATLDYVVPFKVFEGNRPTNSILLREITPFSLGALIALYEHKIFTQGVILNIFTFDQ
WGVELGKQLANRILPELKDDKEISSHDSSTNGLINRYKAWRG*
pgm P36938
(SEQ ID NO: 21)
ATGGCAATCCACAATCGTGCAGGCCAACCTGCACAACAGAGTGATTTGATTAACGTCGC
CCAACTGACGGCGCAATATTATGTACTGAAACCAGAAGCAGGGAATGCGGAGCACGCGGTGAAA
TTCGGTACTTCCGGTCACCGTGGCAGTGCAGCGCGCCACAGCTTTAACGAGCCGCACATTCTGG
CGATCGCTCAGGCAATTGCTGAAGAACGTGCGAAAAACGGCATCACTGGCCCTTGCTATGTGGG
TAAAGATACTCACGCCCTGTCCGAACCTGCATTCATTTCCGTTCTGGAAGTGCTGGCAGCGAAC
GGCGTTGATGTCATTGTGCAGGAAAACAATGGCTTCACCCCGACGCCTGCCGTTTCCAATGCCA
TCCTGGTTCACAATAAAAAAGGTGGCCCGCTGGCAGACGGTATCGTGATTACACCGTCCCATAA
CCCGCCGGAAGATGGTGGAATCAAATACAATCCGCCAAATGGTGGCCCGGCTGATACCAACGTC
ACTAAAGTGGTGGAAGACAGGGCCAACGCACTGCTGGCCGATGGCCTGAAAGGCGTGAAGCGTA
TCTCCCTCGACGAAGCGATGGCATCCGGTCATGTGAAAGAGCAGGATCTGGTGCAGCCGTTCGT
GGAAGGTCTGGCCGATATCGTTGATATGGCCGCGATTCAGAAAGCGGGCCTGACGCTGGGCGTT
GATCCGCTGGGCGGTTCCGGTATCGAATACTGGAAGCGTATTGGCGAGTATTACAACCTCAACC
TGACTATCGTTAACGATCAGGTCGATCAAACCTTCCGCTTTATGCACCTTGATAAAGACGGCGC
GATCCGTATGGACTGCTCCTCCGAGTGTGCGATGGCGGGCCTGCTGGCACTGCGTGATAAGTTC
GATCTGGCGTTTGCTAACGACCCGGATTATGACCGTCACGGTATCGTCACTCCGGCAGGTTTGA
TGAATCCGAACCACTACCTGGCGGTGGCAATCAATTACCTGTTCCAGCATCGTCCGCAGTGGGG
CAAAGATGTTGCCGTCGGTAAAACGCTGGTTTCATCTGCGATGATCGACCGTGTGGTCAACGAC
TTGGGCCGTAAACTGGTAGAAGTCCCGGTAGGTTTCAAATGGTTTGTCGATGGTCTGTTCGACG
GCAGCTTCGGCTTTGGCGGCGAAGAGAGTGCAGGGGCTTCCTTCCTGCGTTTCGACGGCACGCC
GTGGTCCACCGACAAAGACGGCATCATCATGTGTCTGCTGGCGGCGGAAATCACCGCTGTCACC
GGTAAGAACCCGCAGGAACACTACAACGAACTGGCAAAACGCTTTGGTGCGCCGAGCTACAACC
GTTTGCAGGCAGCTGCGACTTCCGCACAAAAAGCGGCGCTGTCTAAGCTGTCTCCGGAAATGGT
GAGCGCCAGCACCCTGGCAGGTGACCCGATCACCGCGCGCCTGACTGCTGCTCCGGGCAACGGT
GCTTCTATTGGCGGTCTGAAAGTGATGACTGACAACGGCTGGTTCGCCGCGCGTCCGTCAGGCA
CGGAAGACGCATATAAGATCTACTGCGAAAGCTTCCTCGGTGAAGAACATCGCAAGCAGATTGA
GAAAGAAGCGGTTGAGATTGTTAGCGAAGTTCTGAAAAACGCGTAA
(SEQ ID NO: 22)
MAIHNRAGQPAQQSDLINVAQLTAQYYVLKPEAGNAEHAVKFGTSGHRGSAARHSFNEP
HILAIAQAIAEERAKNGITGPCYVGKDTHALSEPAFISVLEVLAANGVDVIVQENNGFTPTPAV
SNAILVHNKKGGPLADGIVITPSHNPPEDGGIKYNPPNGGPADTNVTKVVEDRANALLADGLKG
VKRISLDEAMASGHVKEQDLVQPFVEGLADIVDMAAIQKAGLILGVDPLGGSGIEYWKRIGEYY
NLNLTIVNDQVDQTFRFMHLDKDGAIRMDCSSECAMAGLLALRDKFDLAFANDPDYDRHGIVTP
AGLMNPNHYLAVAINYLFQHRPQWGKDVAVGKTLVSSAMIDRVVNDLGRKLVEVPVGFKWFVDG
LFDGSFGFGGEESAGASFLRFDGTPWSTDKDGIIMCLLAAEITAVTGKNPQEHYNELAKRFGAP
SYNRLQAAATSAQKAALSKLSPEMVSASTLAGDPITARLTAAPGNGASIGGLKVMTDNGWFAAR
PSGTEDAYKIYCESFLGEEHRKQIEKEAVEIVSEVLKNA*
galU P0AEP3
(SEQ ID NO: 23)
ATGGCTGCCATTAATACGAAAGTCAAAAAAGCCGTTATCCCCGTTGCGGGATTAGGAAC
CAGGATGTTGCCGGCGACGAAAGCCATCCCGAAAGAGATGCTGCCACTTGTCGATAAGCCATTA
ATTCAATACGTCGTGAATGAATGTATTGCGGCTGGCATTACTGAAATTGTGCTGGTTACACACT
CATCTAAAAACTCTATTGAAAACCACTTTGATACCAGTTTTGAACTGGAAGCAATGCTGGAAAA
ACGTGTAAAACGTCAACTGCTTGATGAAGTGCAGTCTATTTGTCCACCGCACGTGACTATTATG
CAAGTTCGTCAGGGTCTGGCGAAAGGCCTGGGACACGCGGTATTGTGTGCTCACCCGGTAGTGG
GTGATGAACCGGTAGCTGTTATTTTGCCTGATGTTATTCTGGATGAATATGAATCCGATTTGTC
ACAGGATAACCTGGCAGAGATGATCCGCCGCTTTGATGAAACGGGTCATAGCCAGATCATGGTT
GAACCGGTTGCTGATGTGACCGCATATGGCGTTGTGGATTGCAAAGGCGTTGAATTAGCGCCGG
GTGAAAGCGTACCGATGGTTGGTGTGGTAGAAAAACCGAAAGCGGATGTTGCGCCGTCTAATCT
CGCTATTGTGGGTCGTTACGTACTTAGCGCGGATATTTGGCCGTTGCTGGCAAAAACCCCTCCG
GGAGCTGGTGATGAAATTCAGCTCACCGACGCAATTGATATGCTGATCGAAAAAGAAACGGTGG
AAGCCTATCATATGAAAGGGAAGAGCCATGACTGCGGTAATAAATTAGGTTACATGCAGGCCTT
CGTTGAATACGGTATTCGTCATAACACCCTTGGCACGGAATTTAAAGCCTGGCTTGAAGAAGAG
ATGGGCATTAAGAAGTAA
(SEQ ID NO: 24)
MAAINTKVKKAVIPVAGLGTRMLPATKAIPKEMLPLVDKPLIQYVVNECIAAGITEIVL
VTHSSKNSIENHFDTSFELEAMLEKRVKRQLLDEVQSICPPHVTIMQVRQGLAKGLGHAVLCAH
PVVGDEPVAVILPDVILDEYESDLSQDNLAEMIRRFDETGHSQIMVEPVADVTAYGVVDCKGVE
LAPGESVPMVGVVEKPKADVAPSNLAIVGRYVLSADIWPLLAKTPPGAGDEIQLTDAIDMLIEK
ETVEAYHMKGKSHDCGNKLGYMQAFVEYGIRHNTLGTEFKAWLEEEMGIKK*
galE P09147
(SEQ ID NO: 25)
ATGAGAGTTCTGGTTACCGGTGGTAGCGGTTACATTGGAAGTCATACCTGTGTGCAATT
ACTGCAAAACGGTCATGATGTCATCATTCTTGATAACCTCTGTAACAGTAAGCGCAGCGTACTG
CCTGTTATCGAGCGTTTAGGCGGCAAACATCCAACGTTTGTTGAAGGCGATATTCGTAACGAAG
CGTTGATGACCGAGATCCTGCACGATCACGCTATCGACACCGTGATCCACTTCGCCGGGCTGAA
AGCCGTGGGCGAATCGGTACAAAAACCGCTGGAATATTACGACAACAATGTCAACGGCACTCTG
CGCCTGATTAGCGCCATGCGCGCCGCTAACGTCAAAAACTTTATTTTTAGCTCCTCCGCCACCG
TTTATGGCGATCAGCCCAAAATTCCATACGTTGAAAGCTTCCCGACCGGCACACCGCAAAGCCC
TTACGGCAAAAGCAAGCTGATGGTGGAACAGATCCTCACCGATCTGCAAAAAGCCCAGCCGGAC
TGGAGCATTGCCCTGCTGCGCTACTTCAACCCGGTTGGCGCGCATCCGTCGGGCGATATGGGCG
AAGATCCGCAAGGCATTCCGAATAACCTGATGCCATACATCGCCCAGGTTGCTGTAGGCCGTCG
CGACTCGCTGGCGATTTTTGGTAACGATTATCCGACCGAAGATGGTACTGGCGTACGCGATTAC
ATCCACGTAATGGATCTGGCGGACGGTCACGTCGTGGCGATGGAAAAACTGGCGAACAAGCCAG
GCGTACACATCTACAACCTCGGCGCTGGCGTAGGCAACAGCGTGCTGGACGTGGTTAATGCCTT
CAGCAAAGCCTGCGGCAAACCGGTTAATTATCATTTTGCACCGCGTCGCGAGGGCGACCTTCCG
GCCTACTGGGCGGACGCCAGCAAAGCCGACCGTGAACTGAACTGGCGCGTAACGCGCACACTCG
ATGAAATGGCGCAGGACACCTGGCACTGGCAGTCACGCCATCCACAGGGATATCCCGATTAA
(SEQ ID NO: 26)
MRVLVTGGSGYIGSHTCVQLLQNGHDVIILDNLCNSKRSVLPVIERLGGKHPTFVEGDI
RNEALMTEILHDHAIDTVIHFAGLKAVGESVQKPLEYYDNNVNGTLRLISAMRAANVKNFIFSS
SATVYGDQPKIPYVESFPTGTPQSPYGKSKLMVEQILTDLQKAQPDWSIALLRYENPVGAHPSG
DMGEDPQGIPNNLMPYIAQVAVGRRDSLAIFGNDYPTEDGTGVRDYIHVMDLADGHVVAMEKLA
NKPGVHIYNLGAGVGNSVLDVVNAFSKACGKPVNYHFAPRREGDLPAYWADASKADRELNWRVT
RTLDEMAQDTWHWQSRHPQGYPD*
ManA P00946
(SEQ ID NO: 27)
ATGCAAAAACTCATTAACTCAGTGCAAAACTATGCCTGGGGCAGCAAAACGGCGTTGAC
TGAACTTTATGGTATGGAAAATCCGTCCAGCCAGCCGATGGCCGAGCTGTGGATGGGCGCACAT
CCGAAAAGCAGTTCACGAGTGCAGAATGCCGCCGGAGATATCGTTTCACTGCGTGATGTGATTG
AGAGTGATAAATCGACTCTGCTCGGAGAGGCCGTTGCCAAACGCTTTGGCGAACTGCCTTTCCT
GTTCAAAGTATTATGCGCAGCACAGCCACTCTCCATTCAGGTTCATCCAAACAAACACAATTCT
GAAATCGGTTTTGCCAAAGAAAATGCCGCAGGTATCCCGATGGATGCCGCCGAGCGTAACTATA
AAGATCCTAACCACAAGCCGGAGCTGGTTTTTGCGCTGACGCCTTTCCTTGCGATGAACGCGTT
TCGTGAATTTTCCGAGATTGTCTCCCTACTCCAGCCGGTCGCAGGTGCACATCCGGCGATTGCT
CACTTTTTACAACAGCCTGATGCCGAACGTTTAAGCGAACTGTTCGCCAGCCTGTTGAATATGC
AGGGTGAAGAAAAATCCCGCGCGCTGGCGATTTTAAAATCGGCCCTCGATAGCCAGCAGGGTGA
ACCGTGGCAAACGATTCGTTTAATTTCTGAATTTTACCCGGAAGACAGCGGTCTGTTCTCCCCG
CTATTGCTGAATGTGGTGAAATTGAACCCTGGCGAAGCGATGTTCCTGTTCGCTGAAACACCGC
ACGCTTACCTGCAAGGCGTGGCGCTGGAAGTGATGGCAAACTCCGATAACGTGCTGCGTGCGGG
TCTGACGCCTAAATACATTGATATTCCGGAACTGGTTGCCAATGTGAAATTCGAAGCCAAACCG
GCTAACCAGTTGTTGACCCAGCCGGTGAAACAAGGTGCAGAACTGGACTTCCCGATTCCAGTGG
ATGATTTTGCCTTCTCGCTGCATGACCTTAGTGATAAAGAAACCACCATTAGCCAGCAGAGTGC
CGCCATTTTGTTCTGCGTCGAAGGCGATGCAACGTTGTGGAAAGGTTCTCAGCAGTTACAGCTT
AAACCGGGTGAATCAGCGTTTATTGCCGCCAACGAATCACCGGTGACTGTCAAAGGCCACGGCC
GTTTAGCGCGTGTTTACAACAAGCTGTAA
(SEQ ID NO: 28)
MQKLINSVQNYAWGSKTALTELYGMENPSSQPMAELWMGAHPKSSSRVQNAAGDIVSLR
DVIESDKSTLLGEAVAKRFGELPFLFKVLCAAQPLSIQVHPNKHNSEIGFAKENAAGIPMDAAE
RNYKDPNHKPELVFALTPFLAMNAFREFSEIVSLLQPVAGAHPAIAHFLQQPDAERLSELFASL
LNMQGEEKSRALAILKSALDSQQGEPWQTIRLISEFYPEDSGLFSPLLLNVVKLNPGEAMFLFA
ETPHAYLQGVALEVMANSDNVLRAGLIPKYIDIPELVANVKFEAKPANQLLTQPVKQGAELDFP
IPVDDFAFSLHDLSDKETTISQQSAAILFCVEGDATLWKGSQQLQLKPGESAFIAANESPVTVK
GHGRLARVYNKL*
ManB P24175
(SEQ ID NO: 29)
ATGAAAAAATTAACCTGCTTTAAAGCCTATGATATTCGCGGGAAATTAGGCGAAGAACT
GAATGAAGATATCGCCTGGCGCATTGGTCGCGCCTATGGCGAATTTCTCAAACCGAAAACCATT
GTGTTAGGCGGTGATGTCCGCCTCACCAGCGAAACCTTAAAACTGGCGCTGGCGAAAGGTTTAC
AGGATGCGGGCGTTGACGTGCTGGATATTGGTATGTCCGGCACCGAAGAGATCTATTTCGCCAC
GTTCCATCTCGGCGTGGATGGCGGCATTGAAGTTACCGCCAGCCATAATCCGATGGATTATAAC
GGCATGAAGCTGGTTCGCGAGGGGGCTCGCCCGATCAGCGGAGATACCGGACTGCGCGACGTCC
AGCGTCTGGCTGAAGCCAACGACTTTCCTCCCGTCGATGAAACCAAACGCGGTCGCTATCAGCA
AATCAACCTGCGTGACGCTTACGTTGATCACCTGTTCGGTTATATCAATGTCAAAAACCTCACG
CCGCTCAAGCTGGTGATCAACTCCGGGAACGGCGCAGCGGGTCCGGTGGTGGACGCCATTGAAG
CCCGCTTTAAAGCCCTCGGCGCGCCCGTGGAATTAATCAAAGTGCACAACACGCCGGACGGCAA
TTTCCCCAACGGTATTCCTAACCCACTACTGCCGGAATGCCGCGACGACACCCGCAATGCGGTC
ATCAAACACGGCGCGGATATGGGCATTGCTTTTGATGGCGATTTTGACCGCTGTTTCCTGTTTG
ACGAAAAAGGGCAGTTTATTGAGGGCTACTACATTGTCGGCCTGTTGGCAGAAGCATTCCTCGA
AAAAAATCCCGGCGCGAAGATCATCCACGATCCACGTCTCTCCTGGAACACCGTTGATGTGGTG
ACTGCCGCAGGTGGCACGCCGGTAATGTCGAAAACCGGACACGCCTTTATTAAAGAACGTATGC
GCAAGGAAGACGCCATCTATGGTGGCGAAATGAGCGCCCACCATTACTTCCGTGATTTCGCTTA
CTGCGACAGCGGCATGATCCCGTGGCTGCTGGTCGCCGAACTGGTGTGCCTGAAAGATAAAACG
CTGGGCGAACTGGTACGCGACCGGATGGCGGCGTTTCCGGCAAGCGGTGAGATCAACAGCAAAC
TGGCGCAACCCGTTGAGGCGATTAACCGCGTGGAACAGCATTTTAGCCGTGAGGCGCTGGCGGT
GGATCGCACCGATGGCATCAGCATGACCTTTGCCGACTGGCGCTTTAACCTGCGCACCTCCAAT
ACCGAACCGGTGGTGCGCCTGAATGTGGAATCGCGCGGTGATGTGCCGCTGATGGAAGCGCGAA
CGCGAACTCTGCTGACGTTGCTGAACGAGTAA
(SEQ ID NO: 30)
MKKLTCFKAYDIRGKLGEELNEDIAWRIGRAYGEFLKPKTIVLGGDVRLTSETLKLALA
KGLQDAGVDVLDIGMSGTEEIYFATFHLGVDGGIEVTASHNPMDYNGMKLVREGARPISGDTGL
RDVQRLAEANDFPPVDETKRGRYQQINLRDAYVDHLFGYINVKNLTPLKLVINSGNGAAGPVVD
AIEARFKALGAPVELIKVHNTPDGNFPNGIPNPLLPECRDDTRNAVIKHGADMGIAFDGDEDRC
FLFDEKGQFIEGYYIVGLLAEAFLEKNPGAKIIHDPRLSWNTVDVVTAAGGTPVMSKTGHAFIK
ERMRKEDAIYGGEMSAHHYFRDFAYCDSGMIPWLLVAELVCLKDKTLGELVRDRMAAFPASGEI
NSKLAQPVEAINRVEQHFSREALAVDRTDGISMTFADWRFNLRISNTEPVVRLNVESRGDVPLM
EARTRTLLTLLNE*
ManC P24174
(SEQ ID NO: 31)
ATGGCGCAGTCGAAACTCTATCCAGTTGTGATGGCAGGTGGCTCCGGTAGCCGCTTATG
GCCGCTTTCCCGCGTACTTTATCCCAAGCAGTTTTTATGCCTGAAAGGCGATCTCACCATGCTG
CAAACCACCATCTGCCGCCTGAACGGCGTGGAGTGCGAAAGCCCGGTGGTGATTTGCAATGAGC
AGCACCGCTTTATTGTCGCGGAACAGCTGCGTCAACTGAACAAACTTACCGAGAACATTATTCT
CGAACCGGCAGGGCGAAACACGGCACCTGCCATTGCGCTGGCGGCGCTGGCGGCAAAACGTCAT
AGCCCGGAGAGCGACCCGTTAATGCTGGTATTGGCGGCGGATCATGTGATTGCCGATGAAGACG
CGTTCCGTGCCGCCGTGCGTAATGCCATGCCATATGCCGAAGCGGGCAAGCTGGTGACCTTCGG
CATTGTGCCGGATCTACCAGAAACCGGTTATGGCTATATTCGTCGCGGTGAAGTGTCTGCGGGT
GAGCAGGATATGGTGGCCTTTGAAGTGGCGCAGTTTGTCGAAAAACCGAATCTGGAAACCGCTC
AGGCCTATGTGGCAAGCGGCGAATATTACTGGAACAGCGGTATGTTCCTGTTCCGCGCCGGACG
CTATCTCGAAGAACTGAAAAAATATCGCCCGGATATCCTCGATGCCTGTGAAAAAGCGATGAGC
GCCGTCGATCCGGATCTCAATTTTATTCGCGTGGATGAAGAAGCGTTTCTCGCCTGCCCGGAAG
AGTCGGTGGATTACGCGGTCATGGAACGTACGGCAGATGCTGTTGTGGTGCCGATGGATGCGGG
CTGGAGCGATGTTGGCTCCTGGTCTTCATTATGGGAGATCAGCGCCCACACCGCCGAGGGCAAC
GTTTGCCACGGCGATGTGATTAATCACAAAACTGAAAACAGCTATGTGTATGCTGAATCTGGCC
TGGTCACCACCGTCGGGGTGAAAGATCTGGTAGTGGTGCAGACCAAAGATGCGGTGCTGATTGC
CGACCGTAACGCGGTACAGGATGTGAAAAAAGTGGTCGAGCAGATCAAAGCCGATGGTCGCCAT
GAGCATCGGGTGCATCGCGAAGTGTATCGTCCGTGGGGCAAATATGACTCTATCGACGCGGGCG
ACCGCTACCAGGTGAAACGCATCACCGTGAAACCGGGCGAGGGCTTGTCGGTACAGATGCACCA
TCACCGCGCGGAACACTGGGTGGTTGTCGCGGGAACGGCAAAAGTCACCATTGATGGTGATATC
AAACTGCTTGGTGAAAACGAGTCCATTTATATTCCGCTGGGGGCGACGCATTGCCTGGAAAACC
CGGGGAAAATTCCGCTCGATTTAATTGAAGTGCGCTCCGGCTCTTATCTCGAAGAGGATGATGT
GGTGCGTTTCGCGGATCGCTACGGACGGGTGTAA
(SEQ ID NO: 32)
MAQSKLYPVVMAGGSGSRLWPLSRVLYPKQFLCLKGDLIMLQTTICRLNGVECESPVVI
CNEQHRFIVAEQLRQLNKLTENIILEPAGRNTAPAIALAALAAKRHSPESDPLMLVLAADHVIA
DEDAFRAAVRNAMPYAEAGKLVTFGIVPDLPETGYGYIRRGEVSAGEQDMVAFEVAQFVEKPNL
ETAQAYVASGEYYWNSGMFLFRAGRYLEELKKYRPDILDACEKAMSAVDPDLNFIRVDEEAFLA
CPEESVDYAVMERTADAVVVPMDAGWSDVGSWSSLWEISAHTAEGNVCHGDVINHKTENSYVYA
ESGLVTTVGVKDLVVVQTKDAVLIADRNAVQDVKKVVEQIKADGRHEHRVHREVYRPWGKYDSI
DAGDRYQVKRITVKPGEGLSVQMHHHRAEHWVVVAGTAKVTIDGDIKLLGENESIYIPLGATHC
LENPGKIPLDLIEVRSGSYLEEDDVVRFADRYGRV*
Gmd P0AC88
(SEQ ID NO: 33)
ATGTCAAAAGTCGCTCTCATCACCGGTGTAACCGGACAAGACGGTTCTTACCTGGCAGA
GTTTCTGCTGGAAAAAGGTTACGAGGTGCATGGTATTAAGCGTCGCGCATCGTCATTCAACACC
GAGCGCGTGGATCACATTTATCAGGATCCGCACACCTGCAACCCGAAATTCCATCTGCATTATG
GCGACCTGAGTGATACCTCTAACCTGACGCGCATTTTGCGTGAAGTACAGCCGGATGAAGTGTA
CAACCTGGGCGCAATGAGCCACGTTGCGGTCTCTTTTGAGTCACCAGAATATACCGCTGACGTC
GACGCGATGGGTACGCTGCGCCTGCTGGAGGCGATCCGCTTCCTCGGTCTGGAAAAGAAAACTC
GTTTCTATCAGGCTTCCACCTCTGAACTGTATGGTCTGGTGCAGGAAATTCCGCAGAAAGAGAC
CACGCCGTTCTACCCGCGATCTCCGTATGCGGTCGCCAAACTGTACGCCTACTGGATCACCGTT
AACTACCGTGAATCCTACGGCATGTACGCCTGTAACGGAATTCTCTTCAACCATGAATCCCCGC
GCCGCGGCGAAACCTTCGTTACCCGCAAAATCACCCGCGCAATCGCCAACATCGCCCAGGGGCT
GGAGTCGTGCCTGTACCTCGGCAATATGGATTCCCTGCGTGACTGGGGCCACGCCAAAGACTAC
GTAAAAATGCAGTGGATGATGCTGCAGCAGGAACAGCCGGAAGATTTCGTTATCGCGACCGGCG
TTCAGTACTCCGTGCGTCAGTTCGTGGAAATGGCGGCAGCACAGCTGGGCATCAAACTGCGCTT
TGAAGGCACGGGCGTTGAAGAGAAGGGCATTGTGGTTTCCGTCACCGGGCATGACGCGCCGGGC
GTTAAACCGGGTGATGTGATTATCGCTGTTGACCCGCGTTACTTCCGTCCGGCTGAAGTTGAAA
CGCTGCTCGGCGACCCGACCAAAGCGCACGAAAAACTGGGCTGGAAACCGGAAATCACCCTCAG
AGAGATGGTGTCTGAAATGGTGGCTAATGACCTCGAAGCGGCGAAAAAACACTCTCTGCTGAAA
TCTCACGGCTACGACGTGGCGATCGCGCTGGAGTCATAA
(SEQ ID NO: 34)
MSKVALITGVTGQDGSYLAEFLLEKGYEVHGIKRRASSENTERVDHIYQDPHTCNPKFH
LHYGDLSDTSNLTRILREVQPDEVYNLGAMSHVAVSFESPEYTADVDAMGTLRLLEAIRFLGLE
KKTRFYQASTSELYGLVQEIPQKETTPFYPRSPYAVAKLYAYWITVNYRESYGMYACNGILFNH
ESPRRGETFVTRKITRAIANIAQGLESCLYLGNMDSLRDWGHAKDYVKMQWMMLQQEQPEDFVI
ATGVQYSVRQFVEMAAAQLGIKLRFEGTGVEEKGIVVSVTGHDAPGVKPGDVIIAVDPRYFRPA
EVETLLGDPTKAHEKLGWKPEITLREMVSEMVANDLEAAKKHSLLKSHGYDVATALES*
WcaG P32055
(SEQ ID NO: 35)
ATGAGTAAACAACGAGTTTTTATTGCTGGTCATCGCGGGATGGTCGGTTCCGCCATCAA
GCGACAGCTCGAACAGCGCGGTGATGTGGAGCTGGTATTACGCACCCGCGATGAACTGAACCTG
CTGGACAGCCGCGCGGTGCATGATTTCTTTGCCAGCGAAAGCATTGACCAGGTCTATCTGGCGG
CGGCGAAAGTGGGCGGCATTGTTGCCAACAACACGTATCCGGCGGATTTCATCTACCAGAACAT
GATGATTGAGAGCAACATCATTCACGCCGCGCATCAGAACGACGTGAACAAACTGCTGTTTCTC
GGATCGTCCTGTATCTACCCGAAACTGGCAAAACAGCCGATGGCAGAAAGCGAGTTATTGCAGG
GCACGCTGGAGCCGACCAACGAGCCTTATGCCATTGCCAAAATTGCCGGGATCAAACTGTGCGA
ATCTTACAACCGCCAGTACGGACGCGATTACCGCTCAGTCATGCCGACCAACCTGTATGGCCCG
CATGACAACTTCCACCCGAGTAATTCGCATGTGATCCCAGCATTGTTGCGTCGCTTCCACGAGG
CGACGGCACAGAATGCGCCGGACGTTGTGGTATGGGGCAGCGGTACACCGATGCGTGAATTCCT
GCACGTCGATGATATGGCGGCGGCCAGCATTCACGTGATGGAGCTGGCGCACGAAGTCTGGCTG
GAGAACACCCAGCCGATGCTGTCGCACATTAACGTCGGTACCGGTGTTGACTGCACTATCCGCG
AGCTGGCGCAAACCATCGCCAAAGTGGTGGATTACAAAGGTCGGGTGGTTTTTGATGCCAGCAA
GCCGGATGGTACGCCGCGCAAACTGCTGGATGTGACGCGCCTGCATCAGCTTGGCTGGTATCAC
GAAATCTCACTGGAAGCGGGGCTTGCCAGCACTTACCAGTGGTTCCTTGAGAATCGAGACCGCT
TTCGGGGGTAA
(SEQ ID NO: 36)
MSKQRVFIAGHRGMVGSAIKRQLEQRGDVELVLRTRDELNLLDSRAVHDFFASESIDQV
YLAAAKVGGIVANNTYPADFIYQNMMIESNIIHAAHQNDVNKLLFLGSSCIYPKLAKQPMAESE
LLQGTLEPTNEPYAIAKIAGIKLCESYNRQYGRDYRSVMPINLYGPHDNFHPSNSHVIPALLRR
FHEATAQNAPDVVVWGSGTPMREFLHVDDMAAASIHVMELAHEVWLENTQPMLSHINVGTGVDC
TIRELAQTIAKVVDYKGRVVFDASKPDGTPRKLLDVTRLHQLGWYHEISLEAGLASTYQWFLEN
RDRFRG*
glmS AAC76752
(SEQ ID NO: 37)
ATGTGTGGAATTGTTGGCGCGATCGCGCAACGTGATGTAGCAGAAATCCTTCTTGAAGG
TTTACGTCGTCTGGAATACCGCGGATATGACTCTGCCGGTCTGGCCGTTGTTGATGCAGAAGGT
CATATGACCCGCCTGCGTCGCCTCGGTAAAGTCCAGATGCTGGCACAGGCAGCGGAAGAACATC
CTCTGCATGGCGGCACTGGTATTGCTCACACTCGCTGGGCGACCCACGGTGAACCTTCAGAAGT
GAATGCGCATCCGCATGTTTCTGAACACATTGTGGTGGTGCATAACGGCATCATCGAAAACCAT
GAACCGCTGCGTGAAGAGCTAAAAGCGCGTGGCTATACCTTCGTTTCTGAAACCGACACCGAAG
TGATTGCCCATCTGGTGAACTGGGAGCTGAAACAAGGCGGGACTCTGCGTGAGGCCGTTCTGCG
TGCTATCCCGCAGCTGCGTGGTGCGTACGGTACAGTGATCATGGACTCCCGTCACCCGGATACC
CTGCTGGCGGCACGTTCTGGTAGTCCGCTGGTGATTGGCCTGGGGATGGGCGAAAACTTTATCG
CTTCTGACCAGCTGGCGCTGTTGCCGGTGACCCGTCGCTTTATCTTCCTTGAAGAGGGCGATAT
TGCGGAAATCACTCGCCGTTCGGTAAACATCTTCGATAAAACTGGCGCGGAAGTAAAACGTCAG
GATATCGAATCCAATCTGCAATATGACGCGGGCGATAAAGGCATTTACCGTCACTACATGCAGA
AAGAGATCTACGAACAGCCGAACGCGATCAAAAACACCCTTACCGGACGCATCAGCCACGGTCA
GGTTGATTTAAGCGAGCTGGGACCGAACGCCGACGAACTGCTGTCGAAGGTTGAGCATATTCAG
ATCCTCGCCTGTGGTACTTCTTATAACTCCGGTATGGTTTCCCGCTACTGGTTTGAATCGCTAG
CAGGTATTCCGTGCGACGTCGAAATCGCCTCTGAATTCCGCTATCGCAAATCTGCCGTGCGTCG
TAACAGCCTGATGATCACCTTGTCACAGTCTGGCGAAACCGCGGATACCCTGGCTGGCCTGCGT
CTGTCGAAAGAGCTGGGTTACCTTGGTTCACTGGCAATCTGTAACGTTCCGGGTTCTTCTCTGG
TGCGCGAATCCGATCTGGCGCTAATGACCAACGCGGGTACAGAAATCGGCGTGGCATCCACTAA
AGCATTCACCACTCAGTTAACTGTGCTGTTGATGCTGGTGGCGAAGCTGTCTCGCCTGAAAGGT
CTGGATGCCTCCATTGAACATGACATCGTGCATGGTCTGCAGGCGCTGCCGAGCCGTATTGAGC
AGATGCTGTCTCAGGACAAACGCATTGAAGCGCTGGCAGAAGATTTCTCTGACAAACATCACGC
GCTGTTCCTGGGCCGTGGCGATCAGTACCCAATCGCGCTGGAAGGCGCATTGAAGTTGAAAGAG
ATCTCTTACATTCACGCTGAAGCCTACGCTGCTGGCGAACTGAAACACGGTCCGCTGGCGCTAA
TTGATGCCGATATGCCGGTTATTGTTGTTGCACCGAACAACGAATTGCTGGAAAAACTGAAATC
CAACATTGAAGAAGTTCGCGCGCGTGGCGGTCAGTTGTATGTCTTCGCCGATCAGGATGCGGGT
TTTGTAAGTAGCGATAACATGCACATCATCGAGATGCCGCATGTGGAAGAGGTGATTGCACCGA
TCTTCTACACCGTTCCGCTGCAGCTGCTGGCTTACCATGTCGCGCTGATCAAAGGCACCGACGT
TGACCAGCCGCGTAACCTGGCAAAATCGGTTACGGTTGAGTAA
(SEQ ID NO: 38)
MCGIVGAIAQRDVAEILLEGLRRLEYRGYDSAGLAVVDAEGHMTRLRRLGKVQMLAQAA
EEHPLHGGTGIAHTRWATHGEPSEVNAHPHVSEHIVVVHNGIIENHEPLREELKARGYTFVSET
DTEVIAHLVNWELKQGGTLREAVLRAIPQLRGAYGTVIMDSRHPDTLLAARSGSPLVIGLGMGE
NFIASDQLALLPVTRRFIFLEEGDIAEITRRSVNIFDKTGAEVKRQDIESNLQYDAGDKGIYRH
YMQKEIYEQPNAIKNTLTGRISHGQVDLSELGPNADELLSKVEHIQILACGTSYNSGMVSRYWF
ESLAGIPCDVEIASEFRYRKSAVRRNSLMITLSQSGETADTLAGLRLSKELGYLGSLAICNVPG
SSLVRESDLALMTNAGTEIGVASTKAFTTQLTVLLMLVAKLSRLKGLDASIEHDIVHGLQALPS
RIEQMLSQDKRIEALAEDFSDKHHALFLGRGDQYPIALEGALKLKEISYIHAEAYAAGELKHGP
LALIDADMPVIVVAPNNELLEKLKSNIEEVRARGGQLYVFADQDAGFVSSDNMHIIEMPHVEEV
IAPIFYTVPLQLLAYHVALIKGTDVDQPRNLAKSVIVE*
glmM AAC76208
(SEQ ID NO: 39)
ATGAGTAATCGTAAATATTTCGGTACCGATGGGATTCGTGGTCGTGTAGGGGATGCGCC
GATCACACCTGATTTTGTGCTTAAGCTGGGTTGGGCCGCGGGTAAAGTGCTGGCGCGCCACGGC
TCCCGTAAGATTATTATTGGTAAAGACACGCGTATTTCTGGCTATATGCTGGAGTCAGCACTGG
AAGCGGGTCTGGCGGCAGCGGGCCTTTCCGCACTCTTCACTGGCCCGATGCCAACACCGGCCGT
GGCTTATCTGACGCGTACCTTCCGCGCAGAGGCCGGAATTGTGATATCTGCATCGCATAACCCG
TTCTACGATAATGGCATTAAATTCTTCTCTATCGACGGCACCAAACTGCCGGATGCGGTAGAAG
AGGCCATCGAAGCGGAAATGGAAAAGGAGATCAGCTGCGTTGATTCGGCAGAACTGGGTAAAGC
CAGCCGTATCGTTGATGCCGCGGGTCGCTATATCGAGTTTTGCAAAGCCACGTTCCCGAACGAA
CTTAGCCTCAGTGAACTGAAGATTGTGGTGGATTGTGCAAACGGTGCGACTTATCACATCGCGC
CGAACGTGCTGCGCGAACTGGGGGCGAACGTTATCGCTATCGGTTGTGAGCCAAACGGTGTAAA
CATCAATGCCGAAGTGGGGGCTACCGACGTTCGCGCGCTCCAGGCTCGTGTGCTGGCTGAAAAA
GCGGATCTCGGTATTGCCTTCGACGGCGATGGCGATCGCGTGATTATGGTTGACCATGAAGGCA
ATAAAGTCGATGGCGATCAGATCATGTATATCATCGCGCGTGAAGGTCTTCGTCAGGGCCAGCT
GCGTGGTGGCGCTGTGGGTACATTGATGAGCAACATGGGGCTTGAACTGGCGCTGAAACAGTTA
GGAATTCCATTTGCGCGCGCGAAAGTGGGTGACCGCTACGTACTGGAAAAAATGCAGGAGAAAG
GCTGGCGTATCGGTGCAGAGAATTCCGGTCATGTGATCCTGCTGGATAAAACTACTACCGGTGA
CGGCATCGTTGCTGGCTTGCAGGTGCTGGCGGCGATGGCACGTAACCATATGAGCCTGCACGAC
CTTTGCAGCGGCATGAAAATGTTCCCGCAGATTCTGGTTAACGTACGTTACACCGCAGGTAGCG
GCGATCCACTTGAGCATGAGTCAGTTAAAGCCGTGACCGCAGAGGTTGAAGCTGCGCTGGGCAA
CCGTGGACGCGTGTTGCTGCGTAAATCCGGCACCGAACCGTTAATTCGCGTGATGGTGGAAGGC
GAAGACGAAGCGCAGGTGACTGAATTTGCACACCGCATCGCCGATGCAGTAAAAGCCGTTTAA
(SEQ ID NO: 40)
MSNRKYFGTDGIRGRVGDAPITPDFVLKLGWAAGKVLARHGSRKIIIGKDTRISGYMLE
SALEAGLAAAGLSALFTGPMPTPAVAYLTRTFRAEAGIVISASHNPFYDNGIKFFSIDGTKLPD
AVEEAIEAEMEKEISCVDSAELGKASRIVDAAGRYIEFCKATFPNELSLSELKIVVDCANGATY
HIAPNVLRELGANVIAIGCEPNGVNINAEVGATDVRALQARVLAEKADLGIAFDGDGDRVIMVD
HEGNKVDGDQIMYIIAREGLRQGQLRGGAVGTLMSNMGLELALKQLGIPFARAKVGDRYVLEKM
QEKGWRIGAENSGHVILLDKITTGDGIVAGLQVLAAMARNHMSLHDLCSGMKMFPQILVNVRYT
AGSGDPLEHESVKAVTAEVEAALGNRGRVLLRKSGTEPLIRVMVEGEDEAQVTEFAHRIADAVK
AV*
glmU AAC76753
(SEQ ID NO: 41)
ATGTTGAATAATGCTATGAGCGTAGTGATCCTTGCCGCAGGCAAAGGCACGCGCATGTA
TTCCGATCTTCCGAAAGTGCTGCATACCCTTGCCGGGAAAGCGATGGTTCAGCATGTCATTGAT
GCTGCGAATGAATTAGGCGCAGCGCACGTTCACCTGGTGTACGGTCACGGCGGCGATCTGCTAA
AACAGGCGCTGAAAGACGACAACCTTAACTGGGTGCTTCAGGCAGAGCAGCTGGGTACGGGTCA
TGCAATGCAGCAGGCCGCACCTTTCTTTGCCGATGATGAAGACATTTTAATGCTCTACGGCGAC
GTGCCGCTGATCTCTGTCGAAACACTCCAGCGTCTGCGTGATGCTAAACCGCAGGGTGGCATTG
GTCTGCTGACGGTGAAACTGGATGATCCGACCGGTTATGGACGTATCACCCGTGAAAACGGCAA
AGTTACCGGCATTGTTGAGCACAAAGATGCCACCGACGAGCAGCGTCAGATTCAGGAGATCAAC
ACCGGCATTCTGATTGCCAACGGCGCAGATATGAAACGCTGGCTGGCGAAGCTGACCAACAATA
ATGCTCAGGGCGAATACTACATCACCGACATTATTGCGCTGGCGTATCAGGAAGGGCGTGAAAT
CGTCGCCGTTCATCCGCAACGTTTAAGCGAAGTAGAAGGCGTGAATAACCGCCTGCAACTCTCC
CGTCTGGAGCGTGTTTATCAGTCCGAACAGGCTGAAAAACTGCTGTTAGCAGGCGTTATGCTGC
GCGATCCAGCGCGTTTTGATCTGCGTGGTACGCTAACTCACGGGCGCGATGTTGAAATTGATAC
TAACGTTATCATCGAGGGCAACGTGACTCTCGGTCATCGCGTGAAAATTGGCACCGGTTGCGTG
ATTAAAAACAGCGTGATTGGCGATGATTGCGAAATCAGTCCGTATACCGTTGTGGAAGATGCGA
ATCTGGCAGCGGCCTGTACCATTGGCCCGTTTGCCCGTTTGCGTCCTGGTGCTGAGTTGCTGGA
AGGTGCTCACGTCGGTAACTTCGTTGAGATGAAAAAAGCGCGTCTGGGTAAAGGCTCGAAAGCT
GGTCATCTGACTTACCTGGGCGATGCGGAAATTGGCGATAACGTTAACATCGGCGCGGGAACCA
TTACCTGCAACTACGATGGTGCGAATAAATTTAAGACCATTATCGGCGACGATGTGTTTGTTGG
TTCCGACACTCAGCTGGTGGCCCCGGTAACAGTAGGCAAAGGCGCGACCATTGCTGCGGGTACA
ACTGTGACGCGTAATGTCGGCGAAAATGCATTAGCTATCAGCCGTGTGCCGCAGACTCAGAAAG
AAGGCTGGCGTCGTCCGGTAAAGAAAAAGTGA
(SEQ ID NO: 42)
MLNNAMSVVILAAGKGTRMYSDLPKVLHTLAGKAMVQHVIDAANELGAAHVHLVYGHGG
DLLKQALKDDNLNWVLQAEQLGTGHAMQQAAPFFADDEDILMLYGDVPLISVETLQRLRDAKPQ
GGIGLLTVKLDDPTGYGRITRENGKVTGIVEHKDATDEQRQIQEINTGILIANGADMKRWLAKL
TNNNAQGEYYITDIIALAYQEGREIVAVHPQRLSEVEGVNNRLQLSRLERVYQSEQAEKLLLAG
VMLRDPARFDLRGTLTHGRDVEIDINVIIEGNVTLGHRVKIGTGCVIKNSVIGDDCEISPYTVV
EDANLAAACTIGPFARLRPGAELLEGAHVGNFVEMKKARLGKGSKAGHLTYLGDAEIGDNVNIG
AGTITCNYDGANKFKTIIGDDVFVGSDTQLVAPVTVGKGATIAAGTTVTRNVGENALAISRVPQ
TQKEGWRRPVKKK*
GNE Q9Y223
(SEQ ID NO: 43)
ATGGAGAAGAACGGGAACAACCGAAAGCTCCGGGTTTGCGTTGCCACCTGCAACCGAGC
TGACTACTCCAAACTGGCCCCGATCATGTTCGGCATCAAGACAGAGCCCGCGTTCTTTGAGTTG
GACGTGGTGGTGCTCGGCTCCCACCTGATTGACGACTATGGAAACACATACCGCATGATTGAGC
AAGATGACTTTGACATTAACACCAGGCTCCACACGATTGTTAGAGGGGAAGATGAAGCGGCCAT
GGTAGAGTCGGTAGGCCTAGCGCTCGTGAAGCTACCGGACGTCCTCAATCGCCTGAAGCCCGAC
ATCATGATTGTTCACGGAGATCGATTTGACGCCCTTGCTCTGGCTACGTCTGCTGCCTTGATGA
ACATCCGCATCCTTCACATTGAAGGAGGCGAGGTCAGCGGGACCATTGATGACTCTATCAGACA
CGCCATAACAAAACTGGCTCACTACCATGTGTGCTGCACTAGAAGTGCAGAGCAGCACCTGATC
TCTATGTGCGAGGACCACGACCGCATCCTGTTGGCAGGCTGCCCTTCCTATGACAAACTGCTCT
CCGCCAAGAACAAAGACTATATGAGCATCATTCGGATGTGGCTAGGCGATGATGTAAAATGTAA
GGATTACATCGTTGCCCTGCAGCATCCCGTGACCACTGACATTAAGCATTCCATAAAGATGTTT
GAGCTAACACTGGATGCCCTGATCTCGTTTAACAAGAGGACCCTAGTTCTGTTTCCAAATATCG
ATGCAGGCAGCAAGGAGATGGTTCGAGTGATGCGGAAGAAGGGCATCGAGCATCACCCCAATTT
CCGTGCAGTCAAGCACGTCCCGTTTGACCAGTTCATACAGCTGGTCGCCCACGCTGGCTGCATG
ATTGGGAATAGCAGCTGCGGCGTGCGAGAGGTTGGCGCTTTCGGAACACCCGTGATCAACCTGG
GCACAAGGCAGATAGGAAGAGAAACCGGGGAGAATGTTCTTCATGTCAGGGATGCTGACACCCA
AGATAAAATATTGCAAGCACTACACCTCCAGTTCGGCAAACAGTACCCTTGCTCAAAGATATAT
GGGGATGGGAATGCTGTTCCAAGGATTTTAAAGTTTCTCAAATCCATTGACCTTCAAGAGCCAC
TACAGAAGAAATTCTGCTTCCCCCCTGTAAAGGAGAACATCTCTCAAGACATTGACCACATCCT
GGAAACTCTGAGTGCCTTGGCTGTTGATCTTGGCGGGACAAACCTGAGGGTGGCAATAGTTAGC
ATGAAGGGTGAAATCGTTAAGAAGTACACTCAGTTCAACCCTAAAACCTATGAAGAAAGGATTA
GTTTAATCCTGCAGATGTGTGTGGAAGCTGCCGCGGAAGCTGTGAAACTCAATTGCAGAATACT
GGGAGTAGGCATCTCCACAGGTGGCCGCGTGAATCCCCAGGAAGGAGTTGTGCTGCATTCAACC
AAGCTGATCCAGGAATGGAACTCCGTGGACCTCAGGACACCCCTCTCCGACACCCTGCATCTCC
CCGTGTGGGTGGACAATGACGGCAACTGTGCCGCCATGGCAGAGAGGAAGTTCGGCCAAGGAAA
AGGACAGGAGAACTTCGTGACGCTCATCACGGGGACAGGGATCGGTGGGGGGATCATCCACCAG
CACGAACTGATCCACGGCAGCTCCTTCTGCGCGGCGGAGCTCGGCCATCTCGTGGTGTCCCTGG
ACGGTCCTGACTGCTCCTGTGGAAGCCATGGGTGCATCGAAGCGTACGCCTCTGGAATGGCCTT
GCAGAGGGAAGCAAAGAAACTCCATGATGAGGACCTGCTCTTGGTGGAAGGGATGTCAGTACCA
AAAGACGAAGCTGTGGGTGCCCTCCATCTCATCCAGGCTGCCAAGCTGGGCAACGTGAAGGCCC
AGAGCATCTTACGAACAGCTGGAACTGCTTTGGGACTTGGGGTTGTGAACATCCTCCACACTAT
GAATCCTTCCCTGGTGATCCTGTCTGGAGTCCTGGCCAGTCACTACATCCACATCGTCAAGGAC
GTCATCCGCCAGCAAGCCTTGTCCTCCGTGCAGGATGTGGACGTGGTGGTGTCAGACTTGGTGG
ACCCGGCCCTGCTTGGCGCAGCCAGCATGGTTCTGGACTACACAACGCGCAGGATACACTAG
(SEQ ID NO: 44)
MEKNGNNRKLRVCVATCNRADYSKLAPIMFGIKTEPAFFELDVVVLGSHLIDDYGNTYR
MIEQDDFDINTRLHTIVRGEDEAAMVESVGLALVKLPDVLNRLKPDIMIVHGDRFDALALATSA
ALMNIRILHIEGGEVSGTIDDSIRHAITKLAHYHVCCTRSAEQHLISMCEDHDRILLAGCPSYD
KLLSAKNKDYMSIIRMWLGDDVKCKDYIVALQHPVTTDIKHSIKMFELTLDALISFNKRTLVLF
PNIDAGSKEMVRVMRKKGIEHHPNFRAVKHVPFDQFIQLVAHAGCMIGNSSCGVREVGAFGTPV
INLGTRQIGRETGENVLHVRDADTQDKILQALHLQFGKQYPCSKIYGDGNAVPRILKFLKSIDL
QEPLQKKFCFPPVKENISQDIDHILETLSALAVDLGGTNLRVAIVSMKGEIVKKYTQFNPKTYE
ERISLILQMCVEAAAEAVKLNCRILGVGISTGGRVNPQEGVVLHSTKLIQEWNSVDLRTPLSDT
LHLPVWVDNDGNCAAMAERKFGQGKGQENFVTLITGTGIGGGIIHQHELIHGSSFCAAELGHLV
VSLDGPDCSCGSHGCIEAYASGMALQREAKKLHDEDLLLVEGMSVPKDEAVGALHLIQAAKLGN
VKAQSILRTAGTALGLGVVNILHTMNPSLVILSGVLASHYIHIVKDVIRQQALSSVQDVDVVVS
DLVDPALLGAASMVLDYTTRRIH*
NANS Q9NR45
(SEQ ID NO: 45)
ATGCCGCTGGAGCTGGAGCTGTGTCCCGGGCGCTGGGTGGGAGGTCAACACCCGTGCTT
CATCATTGCCGAGATCGGCCAGAACCACCAGGGCGACCTGGACGTAGCCAAGCGCATGATCCGC
ATGGCCAAGGAGTGTGGGGCTGATTGTGCTAAGTTCCAGAAGAGTGAGCTAGAATTTAAGTTTA
ATCGGAAAGCCTTGGAGAGGCCATACACCTCGAAGCATTCCTGGGGGAAAACGTACGGGGAGCA
CAAACGACATCTGGAGTTCAGCCATGACCAGTACAGGGAGCTGCAGAGGTACGCCGAGGAGGTT
GGGATCTTCTTCACTGCCTCTGGCATGGATGAGATGGCAGTTGAATTTCTGCATGAACTGAATG
TTCCATTTTTCAAAGTTGGATCTGGAGACACTAATAATTTTCCTTATCTGGAAAAGACAGCCAA
AAAAGGTCGCCCAATGGTGATCTCCAGTGGGATGCAGTCAATGGACACCATGAAGCAAGTTTAT
CAGATCGTGAAGCCCCTCAACCCCAACTTCTGCTTCTTGCAGTGTACCAGCGCATACCCGCTCC
AGCCTGAGGACGTCAACCTGCGGGTCATCTCGGAATATCAGAAGCTCTTTCCTGACATTCCCAT
AGGGTATTCTGGGCATGAAACAGGCATAGCGATATCTGTGGCCGCAGTGGCTCTGGGGGCCAAG
GTGTTGGAACGTCACATAACTTTGGACAAGACCTGGAAGGGGAGTGACCACTCGGCCTCGCTGG
AGCCTGGAGAACTGGCCGAGCTGGTGCGGTCAGTGCGTCTTGTGGAGCGTGCCCTGGGCTCCCC
AACCAAGCAGCTGCTGCCCTGTGAGATGGCCTGCAATGAGAAGCTGGGCAAGTCTGTGGTGGCC
AAAGTGAAAATTCCGGAAGGCACCATTCTAACAATGGACATGCTCACCGTGAAGGTGGGTGAGC
CCAAAGGCTATCCTCCTGAGGACATCTTTAATCTAGTGGGCAAGAAGGTCCTGGTCACTGTTGA
AGAGGATGACACCATCATGGAAGAATTGGTAGATAATCATGGCAAAAAAATCAAGTCTTAA
(SEQ ID NO: 46)
MPLELELCPGRWVGGQHPCFIIAEIGQNHQGDLDVAKRMIRMAKECGADCAKFQKSELE
FKFNRKALERPYTSKHSWGKTYGEHKRHLEFSHDQYRELQRYAEEVGIFFTASGMDEMAVEFLH
ELNVPFFKVGSGDTNNFPYLEKTAKKGRPMVISSGMQSMDTMKQVYQIVKPLNPNFCFLQCTSA
YPLQPEDVNLRVISEYQKLFPDIPIGYSGHETGIAISVAAVALGAKVLERHITLDKTWKGSDHS
ASLEPGELAELVRSVRLVERALGSPTKQLLPCEMACNEKLGKSVVAKVKIPEGTILTMDMLTVK
VGEPKGYPPEDIFNLVGKKVLVTVEEDDTIMEELVDNHGKKIKS*
NANP Q8TBE9
(SEQ ID NO: 47)
ATGGGGCTGAGCCGCGTGCGGGCGGTTTTCTTTGACTTGGACAACACTCTCATCGACAC
GGCCGGGGCGAGCAGGAGAGGCATGTTGGAGGTGATAAAACTCTTACAATCAAAATACCATTAT
AAAGAAGAGGCTGAAATCATCTGTGATAAAGTTCAAGTTAAACTCAGCAAGGAATGTTTTCATC
CTTACAATACATGCATTACTGATTTAAGGACTTCACATTGGGAAGAAGCAATCCAGGAAACAAA
AGGTGGTGCAGCCAATAGAAAATTGGCTGAAGAATGTTATTTCCTTTGGAAATCTACACGTTTA
CAGCACATGACACTAGCAGAAGATGTCAAAGCCATGCTTACTGAACTTCGAAAGGAGGTCCGCC
TACTTCTATTAACGAATGGGGACAGACAGACCCAGAGGGAGAAGATTGAGGCTTGTGCCTGTCA
GTCCTATTTTGACGCTGTTGTTGTAGGTGGAGAGCAGAGAGAGGAGAAACCAGCACCGTCCATA
TTTTATTACTGCTGCAATCTTCTCGGAGTACAACCTGGGGACTGTGTGATGGTCGGTGACACAT
TAGAAACCGACATCCAAGGAGGCCTCAATGCAGGATTGAAAGCAACAGTCTGGATCAATAAAAA
TGGAATAGTGCCACTGAAGTCCTCCCCAGTTCCGCATTACATGGTTTCTTCTGTGCTAGAGTTA
CCTGCTCTCTTACAAAGTATAGACTGCAAAGTCAGTATGTCCACTTAA
(SEQ ID NO: 48)
MGLSRVRAVFFDLDNTLIDTAGASRRGMLEVIKLLQSKYHYKEEAEIICDKVQVKLSKE
CFHPYNTCITDLRISHWEEAIQETKGGAANRKLAEECYFLWKSTRLQHMTLAEDVKAMLTELRK
EVRLLLLINGDRQTQREKIEACACQSYFDAVVVGGEQREEKPAPSIFYYCCNLLGVQPGDCVMV
GDTLETDIQGGLNAGLKATVWINKNGIVPLKSSPVPHYMVSSVLELPALLQSIDCKVSMST*
CMAS Q8NFW8
(SEQ ID NO: 49)
ATGGACTCGGTGGAGAAGGGTGCTGCTACATCAGTGTCCAACCCGCGTGGACGACCATC
CCGAGGCCGGCCTCCTAAGCTGCAGCGAAACTCTCGCGGCGGCCAGGGCCGAGGTGTGGAGAAG
CCCCCGCACCTGGCAGCCCTAATTCTGGCCCGGGGAGGCAGCAAAGGCATCCCCCTGAAGAACA
TTAAGCACCTGGCGGGGGTCCCGCTCATTGGCTGGGTCCTGCGTGCGGCCCTGGATTCAGGGGC
CTTCCAGAGTGTATGGGTTTCGACAGACCATGATGAAATTGAGAATGTGGCCAAACAATTTGGT
GCACAAGTTCATCGAAGAAGTTCTGAAGTTTCAAAAGACAGCTCTACCTCACTAGATGCCATCA
TAGAATTTCTTAATTATCATAATGAGGTTGACATTGTAGGAAATATTCAAGCTACTTCTCCATG
TTTACATCCTACTGATCTTCAAAAAGTTGCAGAAATGATTCGAGAAGAAGGATATGATTCTGTT
TTCTCTGTTGTGAGGCGCCATCAGTTTCGATGGAGTGAAATTCAGAAAGGAGTTCGTGAAGTGA
CCGAACCTCTGAATTTAAATCCAGCTAAACGGCCTCGTCGACAAGACTGGGATGGAGAATTATA
TGAAAATGGCTCATTTTATTTTGCTAAAAGACATTTGATAGAGATGGGTTACTTGCAGGGTGGA
AAAATGGCATACTACGAAATGCGAGCTGAACATAGTGTGGATATAGATGTGGATATTGATTGGC
CTATTGCAGAGCAAAGAGTATTAAGATATGGCTATTTTGGCAAAGAGAAGCTTAAGGAAATAAA
ACTTTTGGTTTGCAATATTGATGGATGTCTCACCAATGGCCACATTTATGTATCAGGTGACCAA
AAAGAAATAATATCTTATGATGTAAAAGATGCTATTGGGATAAGTTTATTAAAGAAAAGTGGTA
TTGAGGTGAGGCTAATCTCAGAAAGGGCCTGTTCAAAGCAGACGCTGTCATCTTTAAAACTGGA
TTGCAAAATGGAAGTCAGTGTATCAGACAAGCTAGCAGTTGTAGATGAATGGAGAAAAGAAATG
GGCCTGTGCTGGAAAGAAGTGGCATATCTTGGAAATGAAGTGTCTGATGAAGAGTGCTTGAAGA
GAGTGGGCCTAAGTGGCGCTCCTGCTGATGCCTGTTCTACTGCCCAGAAGGCTGTTGGATACAT
TTGCAAATGTAATGGTGGCCGTGGTGCCATCCGAGAATTTGCAGAGCACATTTGCCTACTAATG
GAAAAGGTTAATAATTCATGCCAAAAATAG
(SEQ ID NO: 50)
MDSVEKGAATSVSNPRGRPSRGRPPKLQRNSRGGQGRGVEKPPHLAALILARGGSKGIP
LKNIKHLAGVPLIGWVLRAALDSGAFQSVWVSTDHDEIENVAKQFGAQVHRRSSEVSKDSSTSL
DAIIEFLNYHNEVDIVGNIQATSPCLHPTDLQKVAEMIREEGYDSVFSVVRRHQFRWSEIQKGV
REVTEPLNLNPAKRPRRQDWDGELYENGSFYFAKRHLIEMGYLQGGKMAYYEMRAEHSVDIDVD
IDWPIAEQRVLRYGYFGKEKLKEIKLLVCNIDGCLINGHIYVSGDQKEIISYDVKDAIGISLLK
KSGIEVRLISERACSKQTLSSLKLDCKMEVSVSDKLAVVDEWRKEMGLCWKEVAYLGNEVSDEE
CLKRVGLSGAPADACSTAQKAVGYICKCNGGRGAIREFAEHICLLMEKVNNSCQK*

1. A method of producing a mammalian milk oligosaccharide (MMO) in a plant or plant part, comprising growing a plant comprising and expressing a recombinant MMO biosynthetic pathway sufficient to produce the MMO, and preferably further comprising isolating the MMO from the plant or plant part.

2. A composition comprising a plant or plant part comprising and expressing a recombinant MMO biosynthetic pathway sufficient to produce the MMO.

3. A composition comprising an MMO isolated from a plant or plant part comprising and expressing a recombinant MMO biosynthetic pathway sufficient to produce the MMO, preferably a food product composition comprising a composition, plant or plant part (such as leaves, stems, roots, seeds, fruit or flowers.

4. A method of claim 1, wherein the plant is comprising and expressing a eukaryotic (e.g. human) or prokaryotic (e.g. bacterial, such as E. coli) nucleotide sugar biosynthetic pathway sufficient to increase the production of the MMO, preferably comprising and expressing a prokaryotic (e.g. bacterial, such as E. coli) nucleotide sugar biosynthetic pathway sufficient to increase the production of the MMO, wherein the use of a prokaryotic (as opposed to a eukaryotic) pathway substantially avoids pathway feedback inhibition in the plant.

5. A method of claim 1, wherein the plant is further comprising and expressing a prokaryotic (e.g. bacterial, such as E. coli) nucleotide sugar biosynthetic pathway sufficient to increase the production of the MMO, wherein the pathway comprises one, two or three pathways and corresponding enzymes:
a) GDP-fucose (ManA, ManB, ManC, Gmd, WcaG),
b) UDP-galactose (pgi, pgm, GalU, GalE), and
c) UDP-GlcNAc (glmS, glmM, glmU).

6. A method of claim 1, wherein the MMO is of a mammal selected from human, cow and goat, preferably wherein the MMO is a human milk oligosaccharide (HMO).

7. A method of claim 1, the plant comprising and expressing a plurality of recombinant MMO biosynthetic pathways sufficient to produce a plurality of different MMOs.

8. A method of claim 1, wherein the plant is selected from tobacco, potato, tomato, sorghum, soybean, sugarcane, Arabidopsis, rice, sugar beet, wheat, cassava, and oat.

9. A method of claim 1, wherein the plant or plant part is producing a mammalian milk oligosaccharide (MMO), comprising and expressing a recombinant MMO biosynthetic pathway sufficient to produce the MMO, wherein the pathway comprises recombinant enzymes:
a β1-4 galactosyltransferase (β1-4-GalT; e.g. GalTPM1141), which glycosylates glucose using UDP-galactose to make lactose; and
a β1-3 N-acetylglucosaminyltransferase (β1-3-GnT; e.g. NmLgtA) generates lacto-N-triose (LNTII) through the transfer of GlcNAC from UDP-GlcNAc to the galactose in the lactose; and
at least one of:
a β1-3-galactosyltransferase (β1-3-GalT; e.g. Cvβ3GalT) and a β1-4-GalT (e.g. HP0826 or NmLgtB), convert the LNTII to lacto-N-tetraose (LNT) and lacto-N-neotetraose (LNnT), respectively.

10. A method of claim 1, wherein the plant or plant part is comprising and expressing of a β1-4-GalT (e.g. GalTPM1141), a β1-3-GlcNAcT (e.g. NmLgtA), a β1-4-GalT (e.g. HP0826 or NmLgtB), and a β1-3-GalT (e.g. Cvβ3GalT) (FIG. 1) to produce LNnT and LNT.

11. A method of claim 1, wherein the plant or plant part is producing non-fucosylated neutral MMOs (nMMOs) comprising a lactose core with decorations of galactose and N-acetylglucosamine (GlcNAc).

12. A method of claim 1, wherein the plant or plant part is producing fucosylated MMOs, particularly comprising a lactose core decorated with one or more fucose moieties.

13. A method of claim 1, wherein the plant or plant part is comprising and expressing a β1-4-GalT (e.g. GalTPM1141), a β1-3-GlcNAcT (e.g. NmLgtA), a β1-4-GalT (e.g. HP0826 or NmLgtB), a β1-3-GalT (e.g. Cvβ3GalT) and a α1-2-fucosyltransferase (α1-2-FucT) (e.g. Te2FT) (FIG. 1) to produce lacto-N-fucopentaose I (LNFPI), a fucosylated pentasaccharide generated by the addition of an α-1,2-linked fucose to the terminal galactose in lacto-N-tetraose (LNT).

14. A method of claim 1, wherein the plant or plant part is producing fucosyllactose, lacto-N-tetraose or lacto-N-neotetraose, and lacto-N-fucopentaose I (and particularly, 2′FL, LNT, LNnT and LNFPI) respectively (FIG. 3, Table 1).

15. A method of claim 1, wherein to improve LNFPI yields, nucleotide sugar biosynthetic pathways (FIG. 1B) are expressed to increase the availability of GDP-fucose (ManA, ManB, ManC, Gmd, WcaG), UDP-galactose (pgi, pgm, GalU, GalE), and UDP-GlcNAc (glmS, glmM, glmU) combinatorially (FIG. 4).

16. A method of claim 1, wherein when expressing the LNFP1 biosynthetic pathway (FIG. 1A) and the GDP-fucose biosynthetic pathway (FIG. 1B), there the plant metabolism harbors the innate ability to further glycosylate simple MMOs to create MMOs of higher complexity (such as lactodifucotetraose (LDFT) and lacto-N-difucohexaose I (LNDFHI); FIG. 3, Table 1). that have not been previously produced through any other heterologous hosts.

17. A method of claim 1, producing acidic MMOs, particularly comprising a lactose or nMMO core with one or more N-acetylneuraminic acid (Neu5Ac) moieties, comprising in the plant a mammalian CMP-Neu5Ac pathway, a UDP-GlcNAc pathway and aMMO biosynthetic genes resulting in production of aMMOs, particularly production of CMP-Neu5Ac.

18. A method of claim 1, wherein the plant or plant part is comprising and expressing a three gene bacterial pathway (neuA, neuB, neuC) or a four gene mammalian pathway (GNE, NANS, NANP, CMAS) which utilize UDP-GlcNAc as a precursor for the production of CMP-Neu5Ac.

19. A method of claim 1, wherein the plant or plant part is comprising and expressing a mammalian CMP-Neu5Ac pathway, GalTPM1141, NmLgtA, Cvβ3GalT, Hp0826 (or NmLgtB) and either PmSt3 or St6 (FIG. 1A) to produce 3′SL, 6′SL and isomers of LST.

20. A method of claim 1, wherein the plant is a stable, transgenic plant, comprising constructs for the constitutive production of 2′FL and LNFPI (e.g. in stably transformed N. benthamiana); in embodiments, MMO10 (FIG. 6) contains genes for the four enzymes required to produce lactose, 2′FL, LNTII, LNT, and LNFPI connected via 2A peptides to allow multiple coding sequences to be driven by a single promoter, wherein each transcriptional unit is driven by a strong constitutive promoter to enable MMO production in all tissues, or MMO11 (FIG. 6), which contains a GDP-D-mannose-4,6-dehydratase (Gmd) from the GDP-fucose pathway, wherein both constructs provide the production of multiple MMOs, specifically 2′FL (FIG. 7) and LNFPI (FIG. 8).


SEQUENCE LISTING (50 sequences)

SEQ ID NO: 1 - DNA, 843 bp, Homo sapiens
  source 1..843: mol_type = genomic DNA
atgcccatgacgaactacgtaatatcactaagttcagccagagagcgtaggcgtcatgtgatgaacgaattttccaagcaccacgttccattccagttcttcgacgcagtttcaccatcttctcagctggatttcctcattcaaagacttgtgcctaaccttaacggaacaagccttacaggtggagagaaagggtgccttatctcacatgtggccttatggcataagtgcatacaagacaatctgccatatatagcaatcttcgaagatgacatactccttggcagggacgcccgtgccttcttggcagaggacgagtggctattctctcgattcaactgtgacgatatcttcattatcagactggaaacttttcttcaggaaactatctgcgaagcgctccccaaccccgtgtcatattgcggcagggacttcttggcacttaaagatgaacaccttgggacggctggttatatcatttcacttggggccgccaaatatttattggaaatctttaagaacatggagagtaacaacattttcccaatcgaccacttgattttcaatcgtttcttagcgggtgaagaactgatggtttaccagctatctcctgctctgtgcattcaagagcttcaattgaacgaaaacgagtctttgttggacagccaacttgagtccgaacgcaagaactatcgtcttgctgagaaggctagaaagaagaaaacctggcgtgaaaaggtttaccacatcttcacaaagccacaaagaatgcttaagaaaagaaaggaaagggctgagaagaatgccaaaatgaaactcaaatgcatcgtcaaattcgagtaa

SEQ ID NO: 2 - protein, 280 aa, Homo sapiens
MPMTNYVISLSSARERRRHVMNEFSKHHVPFQFFDAVSPSSQLDFLIQRLVPNLNGTSLTGGEKGCLISHVALWHKCIQDNLPYIAIFEDDILLGRDARAFLAEDEWLFSRFNCDDIFIIRLETFLQETICEALPNPVSYCGRDFLALKDEHLGTAGYIISLGAAKYLLEIFKNMESNNIFPIDHLIFNRFLAGEELMVYQLSPALCIQELQLNENESLLDSQLESERKNYRLAEKARKKKTWREKVYHIFTKPQRMLKKRKERAEKNAKMKLKCIVKFE

SEQ ID NO: 3 - DNA, 999 bp, Homo sapiens
  source 1..999: mol_type = genomic DNA
atgcagcctttagtcagcgtattgatttgcgcctacaacgtagaaaaatatttcgcccaatcattagccgccgtcgtgaatcaaacttggtgcaacttggatattttgattgtcgatgacggctcgacagacggtacgcttgccattgccaaggattttcaaaagcgggacagccgtatcaaaatccttgcacaagctcaaaattccggcctgattccctctttaaacatcgggctggacgaattggcaaagtcgggggggggggaatatattgcacgcaccgatgccgacgatattgccgcccccgactggattgagaaaatcgtgggcgagatggaaaaagaccgcagcatcatcgcgatgggcgcgtggctggaagtgttgtcggaagaaaaggacggcaaccggctggcgcggcatcacaggcacggcaaaatttggaaaaagccgacccgacacgaagatattgccgactttttccctttcggcaaccccatacacaacaacacgatgattatgaggcgcagcgtcattgacggcggtttgcgttacaacaccgagcgggattgggcggaagattaccaattttggtacgatgtcagcaaattgggcaggctggcttattatcccgaagccttggtcaaataccgccttcacgccaatcaggtttcatccaaatacagcatccgccaacacgaaatcgcgcaaggcatccaaaaaaccgccagaaacgattttttgcagtctatgggttttaaaacccggttcgacagccttgaataccgccaaataaaagcagtagcgtatgaattgctggagaaacatttgccggaagaagattttgaacgcgcccgccggtttttgtaccaatgcttcaaacggacggacacgccgcccgccggcgcgtggctggattttgcggcagacggcaggatgcggcggctgtttaccttgaggcaatacttcggcattttgcaccgattgctgaaaaaccgttaa

SEQ ID NO: 4 - protein, 332 aa, Homo sapiens
MQPLVSVLICAYNVEKYFAQSLAAVVNQTWCNLDILIVDDGSTDGTLAIAKDFQKRDSRIKILAQAQNSGLIPSLNIGLDELAKSGGGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILHRLLKNR

SEQ ID NO: 5 - DNA, 789 bp, Homo sapiens
  source 1..789: mol_type = genomic DNA
atggacaccatcatgattaaacgtccgctggttagcgttattctgccggtgaataaaaacaatccgcatctggaagaagcaatccagagcattaaaaaccagacctataaagagctggaactgatcattattgccaacaactgcgaggataacttttatagcctgctgctgaaatatcaggaccagaaaaccaaaattatccgcaccagcatcaaatatctgccgtttagcctgaatctgggtgttcatctgagccagggtgaatatattgcacgtatggattcagatgatatcagcgttctggatcgcattgaaaaacaggttaaacgctttctgaatacaccggaactgagcattctgggtagcaatgttgaatatatcaatgaagccagcgaaagcattggctatagcaactatccgctggatcatagcagcattgttaatagctttccgtttcgttgtaatctggcacatccgaccattatggttaaaaaagaagtgattaccacgcttggtggctatatgtatggtagcctgagcgaagattatgatctgtggattcgtgcaagccgtcatggcaatttcaaatttagcaatattgatgaaccgctgctgaagtaccgtattcataaaggtcaggcaaccaataaaagcaacgcctataacatctttgcctttgatagcagcctgaaaatccgtgaatttctgctgaatggtaatgtgcagtatctgctgggtgcagcacgtggtttttttgcatttctgtatgtgcgtttcatcaaaaaataa

SEQ ID NO: 6 - protein, 262 aa, Homo sapiens
MDTIMIKRPLVSVILPVNKNNPHLEEAIQSIKNQTYKELELIIIANNCEDNFYSLLLKYQDQKTKIIRTSIKYLPFSLNLGVHLSQGEYIARMDSDDISVLDRIEKQVKRFLNTPELSILGSNVEYINEASESIGYSNYPLDHSSIVNSFPFRCNLAHPTIMVKKEVITTLGGYMYGSLSEDYDLWIRASRHGNFKFSNIDEPLLKYRIHKGQATNKSNAYNIFAFDSSLKIREFLLNGNVQYLLGAARGFFAFLYVRFIKK

SEQ ID NO: 7 - DNA, 882 bp, Homo sapiens
  source 1..882: mol_type = genomic DNA
atgattatcgttcacctgtgcggtggcctgggcaaccagatgttccagtacgcagcgggtctggcagcagcgcaccgtatcggttctgaagtgaaattcgacacccattggttcgacgcgacttgtctgcatcagggcctggagctgcgccgtgtttttggtctggaactgccggagccgtcctctaaagacctgcgtaaagttctgggcgcgtgtgttcatccggctgttcgtcgtctgctggctggccacttcctgcatggtctgcgcccgaaatccctggttatccagccgcatttccactactggaccggttttgagcacctgccggacaacgtttacctggagggttactggcagtccgaacgttacttctctaacatcgcggatatcatccgtcagcagtttcgcttcgtggaaccgctggacccgcacaacgcggctctgatggatgaaatgcagtctggtgtgtctgtttctctgcacattcgtcgcggcgattactttaacaacccgcagatgcgtcgcgtgcatggcgtggatctgagcgaatactacccggctgctgttgcgaccatgatcgagaagactaacgcagaacgtttctacgttttctccgacgatccgcagtgggttctggagcatctgaagctgccggttagctataccgttgtggatcacaaccgtggtgctgcgagctaccgtgatatgcagctgatgtccgcgtgccgtcaccacatcatcgcgaactctaccttctcttggtggggtgcgtggctgaacccgcgtccggataaagttgttatcgcaccgcgccactggtttaacgttgacgtttttgacacccgcgatctgtactgcccgggttggattgttctgtaa

SEQ ID NO: 8 - protein, 293 aa, Homo sapiens
MIIVHLCGGLGNQMFQYAAGLAAAHRIGSEVKFDTHWFDATCLHQGLELRRVFGLELPEPSSKDLRKVLGACVHPAVRRLLAGHFLHGLRPKSLVIQPHFHYWTGFEHLPDNVYLEGYWQSERYFSNIADIIRQQFRFVEPLDPHNAALMDEMQSGVSVSLHIRRGDYFNNPQMRRVHGVDLSEYYPAAVATMIEKTNAERFYVFSDDPQWVLEHLKLPVSYTVVDHNRGAASYRDMQLMSACRHHIIANSTFSWWGAWLNPRPDKVVIAPRHWFNVDVFDTRDLYCPGWIVL

SEQ ID NO: 9 - DNA, 828 bp, Homo sapiens
  source 1..828: mol_type = genomic DNA
atgcaaaaccacgttatcagcttagcttccgccgcagaacgcagggcgcacattgccgataccttcggcaggcacggcatcccgtttcagtttttcgacgcactgatgccgtctgaaaggctggaacaggcaatggcggaactcgtccccggcttgtcggcgcacccctatttgagcggagtggaaaaagcctgctttatgagccacgccgtattgtggaagcaggcattggacgaaggtctgccgtatatcaccgtatttgaggacgacgttttactcggcgaaggtgcggaaaaattccttgccgaagacgcttggctgcaagaacgctttgacccggataccgcctttatcgtccgcttggaaacgatgtttatgcacgtcctgacctcgccctccggcgtggcggattactgcgggcgcgcctttccgctgttggaaagcgaacactgggggacggcgggctatatcatttcccgaaaagcgatgcggtttttcctggacaggtttgccgccctgccgcccgaagggctgcaccccgtcgatctgatgatgttcagcgattttttcgacagggaaggaatgccggtttgccagctcaatcccgccttgtgcgcccaagagctgcattatgccaagtttcacgaccaaaacagcgcattgggcagcctgatcgaacacgaccgcctcctgaaccgcaaacagcaaaggcgcgattcccccgccaacacattcaaacaccgcctgatccgcgccttgaccaaaatcagcagggaaagggaaaaacgccggcaaaggcgcgaacagttcattgtgcctttccaataa

SEQ ID NO: 10 - protein, 275 aa, Homo sapiens
MQNHVISLASAAERRAHIADTFGRHGIPFQFFDALMPSERLEQAMAELVPGLSAHPYLSGVEKACFMSHAVLWKQALDEGLPYITVFEDDVLLGEGAEKFLAEDAWLQERFDPDTAFIVRLETMFMHVLTSPSGVADYCGRAFPLLESEHWGTAGYIISRKAMRFFLDRFAALPPEGLHPVDLMMFSDFFDREGMPVCQLNPALCAQELHYAKFHDQNSALGSLIEHDRLLNRKQQRRDSPANTFKHRLIRALTKISREREKRRQRREQFIVPFQ

SEQ ID NO: 11 - DNA, 822 bp, Homo sapiens
  source 1..822: mol_type = genomic DNA
atgcgtgtttttgccatttctttaaatcaaaaagtgtgcgatacatttggtttagtttttagagacaccacaactttactcaatagcatcaatgccacccaccaccaagcgcaaatttttgatgcgatttattctaaaacttttgaaggcgggttgcaccccttagtgaaaaagcatttacacccttatttcatcacgcaaaacatcaaagacatggggattacaaccaatctcatcagtgaggtttctaagttttattacgctttaaaataccatgcgaagtttatgagcttgggggagcttgggtgctatgcgagtcattattccttgtgggaaaaatgcatagaactcaatgaagcgatctgtattttagaagacgatataaccttgaaagaggattttaaagagggcttggattttttagaaaaacacatccaagagttaggctatatccgcttgatgcatttattgtatgatgccagtgtaaaaagtgagccattgagccataaaaaccacgagatacaagagcgtgtggggatcattaaagcttatagcgaaggggtggggactcaaggctatgtgatcacgcctaagattgccaaagtttttttgaaatgcagccgaaaatgggttgttcctgtggatacgataatggacgctacttttatccatggcgtgaaaaatctggtgttacaaccttttgtgatcgctgatgatgagcaaatctctacgatagcacgaaaagaagaaccttatagccctaaaatcgccttaatgagagaactccattttaaatatttgaaatattggcagtttgtataa

SEQ ID NO: 12 - protein, 273 aa, Homo sapiens
MRVFAISLNQKVCDTFGLVFRDTTTLLNSINATHHQAQIFDAIYSKTFEGGLHPLVKKHLHPYFITQNIKDMGITTNLISEVSKFYYALKYHAKFMSLGELGCYASHYSLWEKCIELNEAICILEDDITLKEDFKEGLDFLEKHIQELGYIRLMHLLYDASVKSEPLSHKNHEIQERVGIIKAYSEGVGTQGYVITPKIAKVFLKCSRKWVVPVDTIMDATFIHGVKNLVLQPFVIADDEQISTIARKEEPYSPKIALMRELHFKYLKYWQFV

SEQ ID NO: 13 - DNA, 834 bp, Homo sapiens
  source 1..834: mol_type = genomic DNA
atggataaatttgccgaacatgaaattccgaaagccgttattgttgcaggtaatggtgaaagcctgagccagattgattatcgtctgctgccgaaaaattatgatgtgtttcgctgcaatcagttttattttgaagaacgctattttctgggcaataaaattaaagccgtgttttttacaccgggtgtttttctggaacagtattataccctgtatcatctgaaacgcaataatgaatattttgtggataatgtgattctgagcagctttaatcatccgaccgttgatctggaaaaaagccagaaaattcaggccctgtttattgatgtgattaatggctatgaaaaatatctgagcaaactgaccgcctttgatgtttatctgcgctataaagaactgtatgaaaatcagcgtattaccagcggtgtttatatgtgtgcagttgcaattgcaatgggctataccgatatttatctgaccggcattgatttttatcaggccagcgaagaaaattatgcctttgataataaaaaaccgaatattattcgcctgctgccggattttcgcaaagaaaaaaccctgtttagctatcatagcaaagatattgatctggaagccctgagctttctgcagcagcattatcatgtgaatttttatagcattagcccgatgagtccgctgagcaaacattttccgattccgaccgtggaagatgattgtgaaaccacctttgttgcaccgctgaaagaaaattatattaatgatattctgctgcctccgcattttgtgtatgaaaaactgggcgtcgacaagcttgcggccgcactcgagtga

SEQ ID NO: 14 - protein, 277 aa, Homo sapiens
MDKFAEHEIPKAVIVAGNGESLSQIDYRLLPKNYDVFRCNQFYFEERYFLGNKIKAVFFTPGVFLEQYYTLYHLKRNNEYFVDNVILSSFNHPTVDLEKSQKIQALFIDVINGYEKYLSKLTAFDVYLRYKELYENQRITSGVYMCAVAIAMGYTDIYLTGIDFYQASEENYAFDNKKPNIIRLLPDFRKEKTLFSYHSKDIDLEALSFLQQHYHVNFYSISPMSPLSKHFPIPTVEDDCETTFVAPLKENYINDILLPPHFVYEKLGVDKLAAALE

SEQ ID NO: 15 - DNA, 1116 bp, Homo sapiens
  source 1..1116: mol_type = genomic DNA
atgggcttgaaaaaggcttgtttgaccgtgttgtgtttgattgttttttgtttcgggatattttatacatttgaccgggtaaatcagggggaaaggaatgcggtttccctgctgaaggagaaacttttcaatgaagagggggaaccggtcaatctgattttctgttataccatattgcagatgaaggtggcggaaaggattatggcgcagcatccgggcgagcggttttatgtggtgctgatgtctgaaaacaggaatgaaaaatacgattattatttcaatcagataaaggataaggcggagcgggcgtactttttccacctgccctacggtttgaacaaatcgtttaatttcattccgacgatggcggagctgaaggtaaagtcgatgctgctgccgaaagtcaagcggatttatttggcaagtttggaaaaagtcagcattgccgcctttttgagcacttacccggatgcggaaatcaaaacctttgacgacgggacaggcaatttaattcaaagcagcagctatttgggcgatgagttttctgtaaacgggacgatcaagcggaattttgcccggatgatgatcggagattggagcatcgccaaaacccgcaatgcttccgacgagcattacacgatattcaagggtttgaaaaacattatggacgacggccgccgcaagatgacttacctgccgctgttcgatgcgtccgaactgaagacgggggacgaaacgggcggcacggtgcggatacttttgggttcgcccgacaaagagatgaaggaaatttcggaaaaggcggcaaaaaacttcaaaatacaatatgtcgcgccgcatccccgccaaacctacgggctttccggcgtaaccacattaaattcgccctatgtcatcgaagactatattttgcgcgagattaagaaaaacccgcatacgaggtatgaaatttatacctttttcagcggcgcggcgttgacgatgaaggattttcccaatgtgcacgtttacgcattgaaaccggcttcccttccggaagattattggctcaagccggtgtatgccctgtttacccaatccggcatcccgattttgacatttgacgataaaaattaa

SEQ ID NO: 16 - protein, 371 aa, Homo sapiens
MGLKKACLTVLCLIVFCFGIFYTFDRVNQGERNAVSLLKEKLFNEEGEPVNLIFCYTILQMKVAERIMAQHPGERFYVVLMSENRNEKYDYYFNQIKDKAERAYFFHLPYGLNKSFNFIPTMAELKVKSMLLPKVKRIYLASLEKVSIAAFLSTYPDAEIKTFDDGTGNLIQSSSYLGDEFSVNGTIKRNFARMMIGDWSIAKTRNASDEHYTIFKGLKNIMDDGRRKMTYLPLFDASELKTGDETGGTVRILLGSPDKEMKEISEKAAKNFKIQYVAPHPRQTYGLSGVTTLNSPYVIEDYILREIKKNPHTRYEIYTFFSGAALTMKDFPNVHVYALKPASLPEDYWLKPVYALFTQSGIPILTFDDKN

SEQ ID NO: 17 - DNA, 1545 bp, Homo sapiens
  source 1..1545: mol_type = genomic DNA
atgaaaaactttttattattaactttaatattacttactgcttgtaataattcagaagaaaatacacaatctattattaaaaatgatattaataaaactattattgatgaggagtatgttaatttagagccaattaatcaatcaaacatctcttttacaaaacactcttgggtacaaacttgtggtacgcaacaactattaacagaacaaaataaagagtcaatatcattatctgtagtggcgccacgattagatgacgatgaaaagtactgctttgattttaatggtgttagtaataaaggtgaaaaatatataacaaaagtaacattaaacgtagtggctccatctttagaggtttatgttgatcatgcatctcttccaactcttcagcagctaatggatattattaaatcggaagaagaaaatcctacagcacaaagatatatagcttgggggagaatagttccgactgatgagcaaatgaaagagttaaatattacatcgtttgcattgataaataaccatacaccagctgacttagtacaagaaattgttaagcaagcacaaacaaagcatagattgaatgttaaacttagctctaacactgctcattcatttgataatttagtgccaatactaaaagaattaaattcgtttaataacgttacggtaacaaatatagatttatatgatgatggttcagcagaatatgtaaatttatataactggagagatacattaaataaaacagataatttaaaaattggtaaagattatcttgaggatgtcattaatggtatcaatgaagacacttcaaatacaggaacatcatctgtttataactggcaaaaactatatccagctaactaccattttttaagaaaagattatttaactttagaaccatcattacatgagttacgagactatattggtgatagtttaaagcaaatgcaatgggatggtttcaaaaaattcaatagcaaacaacaagaattattcttatcgattgttaattttgacaaacaaaaattacaaaatgaatataattcatctaatttaccaaactttgtgtttacaggtacgactgtatgggctggtaaccatgaaagagagtattatgcgaaacaacaaattaatgtcattaataatgcaattaatgaatcgagcccacattatttaggcaatagttatgatttgttcttcaaaggtcaccctggtggcggtatcattaatacattaataatgcaaaactatccttcaatggttgatattccatcaaaaatatcatttgaagttttgatgatgacagatatgcttcctgatgcagttgctggtatagcgagctctttatatttcacgataccagctgaaaaaattaaatttatagtttttacatcgacagaaactataactgatcgtgaaactgctttgagaagtcctttagttcaagtaatgataaaactaggtattgtaaaagaagagaatgtacttttttgggctgatctgccaaattgtgaaacaggtgtttgtattgcagtctag

SEQ ID NO: 18 - protein, 514 aa, Homo sapiens
MKNFLLLTLILLTACNNSEENTQSIIKNDINKTIIDEEYVNLEPINQSNISFTKHSWVQTCGTQQLLTEQNKESISLSVVAPRLDDDEKYCFDFNGVSNKGEKYITKVTLNVVAPSLEVYVDHASLPTLQQLMDIIKSEEENPTAQRYIAWGRIVPTDEQMKELNITSFALINNHTPADLVQEIVKQAQTKHRLNVKLSSNTAHSFDNLVPILKELNSFNNVTVTNIDLYDDGSAEYVNLYNWRDTLNKTDNLKIGKDYLEDVINGINEDTSNTGTSSVYNWQKLYPANYHFLRKDYLTLEPSLHELRDYIGDSLKQMQWDGFKKFNSKQQELFLSIVNFDKQKLQNEYNSSNLPNFVFTGTTVWAGNHEREYYAKQQINVINNAINESSPHYLGNSYDLFFKGHPGGGIINTLIMQNYPSMVDIPSKISFEVLMMTDMLPDAVAGIASSLYFTIPAEKIKFIVFTSTETITDRETALRSPLVQVMIKLGIVKEENVLFWADLPNCETGVCIAV

SEQ ID NO: 19 - DNA, 1650 bp, Homo sapiens
  source 1..1650: mol_type = genomic DNA
atgaaaaacatcaatccaacgcagaccgctgcctggcaggcactacagaaacacttcgatgaaatgaaagacgttacgatcgccgatctttttgctaaagacggcgatcgtttttctaagttctccgcaaccttcgacgatcagatgctggtggattactccaaaaaccgcatcactgaagagacgctggcgaaattacaggatctggcgaaagagtgcgatctggcgggcgcgattaagtcgatgttctctggcgagaagatcaaccgcactgaaaaccgcgccgtgctgcacgtagcgctgcgtaaccgtagcaataccccgattttggttgatggcaaagacgtaatgccggaagtcaacgcggtgctggagaagatgaaaaccttctcagaagcgattatttccggtgagtggaaaggttataccggcaaagcaatcactgacgtagtgaacatcgggatcggcggttctgacctcggcccatacatggtgaccgaagctctgcgtccgtacaaaaaccacctgaacatgcactttgtttctaacgtcgatgggactcacatcgcggaagtgctgaaaaaagtaaacccggaaaccacgctgttcttggtagcatctaaaaccttcaccactcaggaaactatgaccaacgcccatagcgcgcgtgactggttcctgaaagcggcaggtgatgaaaaacacgttgcaaaacactttgcggcgctttccaccaatgccaaagccgttggcgagtttggtattgatactgccaacatgttcgagttctgggactgggttggcggccgttactctttgtggtcagcgattggcctgtcgattgttctctccatcggctttgataacttcgttgaactgctttccggcgcacacgcgatggacaagcatttctccaccacgcctgccgagaaaaacctgcctgtactgctggcgctgattggcatctggtacaacaatttctttggtgcggaaactgaagcgattctgccgtatgaccagtatatgcaccgtttcgcggcgtacttccagcagggcaatatggagtccaacggtaagtatgttgaccgtaacggtaacgttgtggattaccagactggcccgattatctggggtgaaccaggcactaacggtcagcacgcgttctaccagctgatccaccagggaaccaaaatggtaccgtgcgatttcatcgctccggctatcacccataacccgctctctgatcatcaccagaaactgctgtctaacttcttcgcccagaccgaagcgctggcgtttggtaaatcccgcgaagtggttgagcaggaatatcgtgatcagggtaaagatccggcaacgcttgactacgtggtgccgttcaaagtattcgaaggtaaccgcccgaccaactccatcctgctgcgtgaaatcactccgttcagcctgggtgcgttgattgcgctgtatgagcacaaaatctttactcagggcgtgatcctgaacatcttcaccttcgaccagtggggcgtggaactgggtaaacagctggcgaaccgtattctgccagagctgaaagatgataaagaaatcagcagccacgatagctcgaccaatggtctgattaaccgctataaagcgtggcgcggttaa

SEQ ID NO: 20 - protein, 549 aa, Homo sapiens
MKNINPTQTAAWQALQKHFDEMKDVTIADLFAKDGDRFSKFSATFDDQMLVDYSKNRITEETLAKLQDLAKECDLAGAIKSMFSGEKINRTENRAVLHVALRNRSNTPILVDGKDVMPEVNAVLEKMKTFSEAIISGEWKGYTGKAITDVVNIGIGGSDLGPYMVTEALRPYKNHLNMHFVSNVDGTHIAEVLKKVNPETTLFLVASKTFTTQETMTNAHSARDWFLKAAGDEKHVAKHFAALSTNAKAVGEFGIDTANMFEFWDWVGGRYSLWSAIGLSIVLSIGFDNFVELLSGAHAMDKHFSTTPAEKNLPVLLALIGIWYNNFFGAETEAILPYDQYMHRFAAYFQQGNMESNGKYVDRNGNVVDYQTGPIIWGEPGTNGQHAFYQLIHQGTKMVPCDFIAPAITHNPLSDHHQKLLSNFFAQTEALAFGKSREVVEQEYRDQGKDPATLDYVVPFKVFEGNRPTNSILLREITPFSLGALIALYEHKIFTQGVILNIFTFDQWGVELGKQLANRILPELKDDKEISSHDSSTNGLINRYKAWRG

SEQ ID NO: 21 - DNA, 1641 bp, Homo sapiens
  source 1..1641: mol_type = genomic DNA
atggcaatccacaatcgtgcaggccaacctgcacaacagagtgatttgattaacgtcgcccaactgacggcgcaatattatgtactgaaaccagaagcagggaatgcggagcacgcggtgaaattcggtacttccggtcaccgtggcagtgcagcgcgccacagctttaacgagccgcacattctggcgatcgctcaggcaattgctgaagaacgtgcgaaaaacggcatcactggcccttgctatgtgggtaaagatactcacgccctgtccgaacctgcattcatttccgttctggaagtgctggcagcgaacggcgttgatgtcattgtgcaggaaaacaatggcttcaccccgacgcctgccgtttccaatgccatcctggttcacaataaaaaaggtggcccgctggcagacggtatcgtgattacaccgtcccataacccgccggaagatggtggaatcaaatacaatccgccaaatggtggcccggctgataccaacgtcactaaagtggtggaagacagggccaacgcactgctggccgatggcctgaaaggcgtgaagcgtatctccctcgacgaagcgatggcatccggtcatgtgaaagagcaggatctggtgcagccgttcgtggaaggtctggccgatatcgttgatatggccgcgattcagaaagcgggcctgacgctgggcgttgatccgctgggcggttccggtatcgaatactggaagcgtattggcgagtattacaacctcaacctgactatcgttaacgatcaggtcgatcaaaccttccgctttatgcaccttgataaagacggcgcgatccgtatggactgctcctccgagtgtgcgatggcgggcctgctggcactgcgtgataagttcgatctggcgtttgctaacgacccggattatgaccgtcacggtatcgtcactccggcaggtttgatgaatccgaaccactacctggcggtggcaatcaattacctgttccagcatcgtccgcagtggggcaaagatgttgccgtcggtaaaacgctggtttcatctgcgatgatcgaccgtgtggtcaacgacttgggccgtaaactggtagaagtcccggtaggtttcaaatggtttgtcgatggtctgttcgacggcagcttcggctttggcggcgaagagagtgcaggggcttccttcctgcgtttcgacggcacgccgtggtccaccgacaaagacggcatcatcatgtgtctgctggcggcggaaatcaccgctgtcaccggtaagaacccgcaggaacactacaacgaactggcaaaacgctttggtgcgccgagctacaaccgtttgcaggcagctgcgacttccgcacaaaaagcggcgctgtctaagctgtctccggaaatggtgagcgccagcaccctggcaggtgacccgatcaccgcgcgcctgactgctgctccgggcaacggtgcttctattggcggtctgaaagtgatgactgacaacggctggttcgccgcgcgtccgtcaggcacggaagacgcatataagatctactgcgaaagcttcctcggtgaagaacatcgcaagcagattgagaaagaagcggttgagattgttagcgaagttctgaaaaacgcgtaa

SEQ ID NO: 22 - protein, 546 aa, Homo sapiens
MAIHNRAGQPAQQSDLINVAQLTAQYYVLKPEAGNAEHAVKFGTSGHRGSAARHSFNEPHILAIAQAIAEERAKNGITGPCYVGKDTHALSEPAFISVLEVLAANGVDVIVQENNGFTPTPAVSNAILVHNKKGGPLADGIVITPSHNPPEDGGIKYNPPNGGPADTNVTKVVEDRANALLADGLKGVKRISLDEAMASGHVKEQDLVQPFVEGLADIVDMAAIQKAGLTLGVDPLGGSGIEYWKRIGEYYNLNLTIVNDQVDQTFRFMHLDKDGAIRMDCSSECAMAGLLALRDKFDLAFANDPDYDRHGIVTPAGLMNPNHYLAVAINYLFQHRPQWGKDVAVGKTLVSSAMIDRVVNDLGRKLVEVPVGFKWFVDGLFDGSFGFGGEESAGASFLRFDGTPWSTDKDGIIMCLLAAEITAVTGKNPQEHYNELAKRFGAPSYNRLQAAATSAQKAALSKLSPEMVSASTLAGDPITARLTAAPGNGASIGGLKVMTDNGWFAARPSGTEDAYKIYCESFLGEEHRKQIEKEAVEIVSEVLKNA

SEQ ID NO: 23 - DNA, 909 bp, Homo sapiens
  source 1..909: mol_type = genomic DNA
atggctgccattaatacgaaagtcaaaaaagccgttatccccgttgcgggattaggaaccaggatgttgccggcgacgaaagccatcccgaaagagatgctgccacttgtcgataagccattaattcaatacgtcgtgaatgaatgtattgcggctggcattactgaaattgtgctggttacacactcatctaaaaactctattgaaaaccactttgataccagttttgaactggaagcaatgctggaaaaacgtgtaaaacgtcaactgcttgatgaagtgcagtctatttgtccaccgcacgtgactattatgcaagttcgtcagggtctggcgaaaggcctgggacacgcggtattgtgtgctcacccggtagtgggtgatgaaccggtagctgttattttgcctgatgttattctggatgaatatgaatccgatttgtcacaggataacctggcagagatgatccgccgctttgatgaaacgggtcatagccagatcatggttgaaccggttgctgatgtgaccgcatatggcgttgtggattgcaaaggcgttgaattagcgccgggtgaaagcgtaccgatggttggtgtggtagaaaaaccgaaagcggatgttgcgccgtctaatctcgctattgtgggtcgttacgtacttagcgcggatatttggccgttgctggcaaaaacccctccgggagctggtgatgaaattcagctcaccgacgcaattgatatgctgatcgaaaaagaaacggtggaagcctatcatatgaaagggaagagccatgactgcggtaataaattaggttacatgcaggccttcgttgaatacggtattcgtcataacacccttggcacggaatttaaagcctggcttgaagaagagatgggcattaagaagtaa

SEQ ID NO: 24 - protein, 302 aa, Homo sapiens
MAAINTKVKKAVIPVAGLGTRMLPATKAIPKEMLPLVDKPLIQYVVNECIAAGITEIVLVTHSSKNSIENHFDTSFELEAMLEKRVKRQLLDEVQSICPPHVTIMQVRQGLAKGLGHAVLCAHPVVGDEPVAVILPDVILDEYESDLSQDNLAEMIRRFDETGHSQIMVEPVADVTAYGVVDCKGVELAPGESVPMVGVVEKPKADVAPSNLAIVGRYVLSADIWPLLAKTPPGAGDEIQLTDAIDMLIEKETVEAYHMKGKSHDCGNKLGYMQAFVEYGIRHNTLGTEFKAWLEEEMGIKK

SEQ ID NO: 25 - DNA, 1017 bp, Homo sapiens
  source 1..1017: mol_type = genomic DNA
atgagagttctggttaccggtggtagcggttacattggaagtcatacctgtgtgcaattactgcaaaacggtcatgatgtcatcattcttgataacctctgtaacagtaagcgcagcgtactgcctgttatcgagcgtttaggcggcaaacatccaacgtttgttgaaggcgatattcgtaacgaagcgttgatgaccgagatcctgcacgatcacgctatcgacaccgtgatccacttcgccgggctgaaagccgtgggcgaatcggtacaaaaaccgctggaatattacgacaacaatgtcaacggcactctgcgcctgattagcgccatgcgcgccgctaacgtcaaaaactttatttttagctcctccgccaccgtttatggcgatcagcccaaaattccatacgttgaaagcttcccgaccggcacaccgcaaagcccttacggcaaaagcaagctgatggtggaacagatcctcaccgatctgcaaaaagcccagccggactggagcattgccctgctgcgctacttcaacccggttggcgcgcatccgtcgggcgatatgggcgaagatccgcaaggcattccgaataacctgatgccatacatcgcccaggttgctgtaggccgtcgcgactcgctggcgatttttggtaacgattatccgaccgaagatggtactggcgtacgcgattacatccacgtaatggatctggcggacggtcacgtcgtggcgatggaaaaactggcgaacaagccaggcgtacacatctacaacctcggcgctggcgtaggcaacagcgtgctggacgtggttaatgccttcagcaaagcctgcggcaaaccggttaattatcattttgcaccgcgtcgcgagggcgaccttccggcctactgggcggacgccagcaaagccgaccgtgaactgaactggcgcgtaacgcgcacactcgatgaaatggcgcaggacacctggcactggcagtcacgccatccacagggatatcccgattaa

SEQ ID NO: 26 - protein, 338 aa, Homo sapiens
MRVLVTGGSGYIGSHTCVQLLQNGHDVIILDNLCNSKRSVLPVIERLGGKHPTFVEGDIRNEALMTEILHDHAIDTVIHFAGLKAVGESVQKPLEYYDNNVNGTLRLISAMRAANVKNFIFSSSATVYGDQPKIPYVESFPTGTPQSPYGKSKLMVEQILTDLQKAQPDWSIALLRYFNPVGAHPSGDMGEDPQGIPNNLMPYIAQVAVGRRDSLAIFGNDYPTEDGTGVRDYIHVMDLADGHVVAMEKLANKPGVHIYNLGAGVGNSVLDVVNAFSKACGKPVNYHFAPRREGDLPAYWADASKADRELNWRVTRTLDEMAQDTWHWQSRHPQGYPD

SEQ ID NO: 27 - DNA, 1176 bp, Homo sapiens
  source 1..1176: mol_type = genomic DNA
atgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaa

SEQ ID NO: 28 - protein, 391 aa, Homo sapiens
MQKLINSVQNYAWGSKTALTELYGMENPSSQPMAELWMGAHPKSSSRVQNAAGDIVSLRDVIESDKSTLLGEAVAKRFGELPFLFKVLCAAQPLSIQVHPNKHNSEIGFAKENAAGIPMDAAERNYKDPNHKPELVFALTPFLAMNAFREFSEIVSLLQPVAGAHPAIAHFLQQPDAERLSELFASLLNMQGEEKSRALAILKSALDSQQGEPWQTIRLISEFYPEDSGLFSPLLLNVVKLNPGEAMFLFAETPHAYLQGVALEVMANSDNVLRAGLTPKYIDIPELVANVKFEAKPANQLLTQPVKQGAELDFPIPVDDFAFSLHDLSDKETTISQQSAAILFCVEGDATLWKGSQQLQLKPGESAFIAANESPVTVKGHGRLARVYNKL

SEQ ID NO: 29 - DNA, 1371 bp, Homo sapiens
  source 1..1371: mol_type = genomic DNA
atgaaaaaattaacctgctttaaagcctatgatattcgcgggaaattaggcgaagaactgaatgaagatatcgcctggcgcattggtcgcgcctatggcgaatttctcaaaccgaaaaccattgtgttaggcggtgatgtccgcctcaccagcgaaaccttaaaactggcgctggcgaaaggtttacaggatgcgggcgttgacgtgctggatattggtatgtccggcaccgaagagatctatttcgccacgttccatctcggcgtggatggcggcattgaagttaccgccagccataatccgatggattataacggcatgaagctggttcgcgagggggctcgcccgatcagcggagataccggactgcgcgacgtccagcgtctggctgaagccaacgactttcctcccgtcgatgaaaccaaacgcggtcgctatcagcaaatcaacctgcgtgacgcttacgttgatcacctgttcggttatatcaatgtcaaaaacctcacgccgctcaagctggtgatcaactccgggaacggcgcagcgggtccggtggtggacgccattgaagcccgctttaaagccctcggcgcgcccgtggaattaatcaaagtgcacaacacgccggacggcaatttccccaacggtattcctaacccactactgccggaatgccgcgacgacacccgcaatgcggtcatcaaacacggcgcggatatgggcattgcttttgatggcgattttgaccgctgtttcctgtttgacgaaaaagggcagtttattgagggctactacattgtcggcctgttggcagaagcattcctcgaaaaaaatcccggcgcgaagatcatccacgatccacgtctctcctggaacaccgttgatgtggtgactgccgcaggtggcacgccggtaatgtcgaaaaccggacacgcctttattaaagaacgtatgcgcaaggaagacgccatctatggtggcgaaatgagcgcccaccattacttccgtgatttcgcttactgcgacagcggcatgatcccgtggctgctggtcgccgaactggtgtgcctgaaagataaaacgctgggcgaactggtacgcgaccggatggcggcgtttccggcaagcggtgagatcaacagcaaactggcgcaacccgttgaggcgattaaccgcgtggaacagcattttagccgtgaggcgctggcggtggatcgcaccgatggcatcagcatgacctttgccgactggcgctttaacctgcgcacctccaataccgaaccggtggtgcgcctgaatgtggaatcgcgcggtgatgtgccgctgatggaagcgcgaacgcgaactctgctgacgttgctgaacgagtaa

SEQ ID NO: 30 - protein, 456 aa, Homo sapiens
MKKLTCFKAYDIRGKLGEELNEDIAWRIGRAYGEFLKPKTIVLGGDVRLTSETLKLALAKGLQDAGVDVLDIGMSGTEEIYFATFHLGVDGGIEVTASHNPMDYNGMKLVREGARPISGDTGLRDVQRLAEANDFPPVDETKRGRYQQINLRDAYVDHLFGYINVKNLTPLKLVINSGNGAAGPVVDAIEARFKALGAPVELIKVHNTPDGNFPNGIPNPLLPECRDDTRNAVIKHGADMGIAFDGDFDRCFLFDEKGQFIEGYYIVGLLAEAFLEKNPGAKIIHDPRLSWNTVDVVTAAGGTPVMSKTGHAFIKERMRKEDAIYGGEMSAHHYFRDFAYCDSGMIPWLLVAELVCLKDKTLGELVRDRMAAFPASGEINSKLAQPVEAINRVEQHFSREALAVDRTDGISMTFADWRFNLRTSNTEPVVRLNVESRGDVPLMEARTRTLLTLLNE

SEQ ID NO: 31 - DNA, 1437 bp, Homo sapiens
  source 1..1437: mol_type = genomic DNA
atggcgcagtcgaaactctatccagttgtgatggcaggtggctccggtagccgcttatggccgctttcccgcgtactttatcccaagcagtttttatgcctgaaaggcgatctcaccatgctgcaaaccaccatctgccgcctgaacggcgtggagtgcgaaagcccggtggtgatttgcaatgagcagcaccgctttattgtcgcggaacagctgcgtcaactgaacaaacttaccgagaacattattctcgaaccggcagggcgaaacacggcacctgccattgcgctggcggcgctggcggcaaaacgtcatagcccggagagcgacccgttaatgctggtattggcggcggatcatgtgattgccgatgaagacgcgttccgtgccgccgtgcgtaatgccatgccatatgccgaagcgggcaagctggtgaccttcggcattgtgccggatctaccagaaaccggttatggctatattcgtcgcggtgaagtgtctgcgggtgagcaggatatggtggcctttgaagtggcgcagtttgtcgaaaaaccgaatctggaaaccgctcaggcctatgtggcaagcggcgaatattactggaacagcggtatgttcctgttccgcgccggacgctatctcgaagaactgaaaaaatatcgcccggatatcctcgatgcctgtgaaaaagcgatgagcgccgtcgatccggatctcaattttattcgcgtggatgaagaagcgtttctcgcctgcccggaagagtcggtggattacgcggtcatggaacgtacggcagatgctgttgtggtgccgatggatgcgggctggagcgatgttggctcctggtcttcattatgggagatcagcgcccacaccgccgagggcaacgtttgccacggcgatgtgattaatcacaaaactgaaaacagctatgtgtatgctgaatctggcctggtcaccaccgtcggggtgaaagatctggtagtggtgcagaccaaagatgcggtgctgattgccgaccgtaacgcggtacaggatgtgaaaaaagtggtcgagcagatcaaagccgatggtcgccatgagcatcgggtgcatcgcgaagtgtatcgtccgtggggcaaatatgactctatcgacgcgggcgaccgctaccaggtgaaacgcatcaccgtgaaaccgggcgagggcttgtcggtacagatgcaccatcaccgcgcggaacactgggtggttgtcgcgggaacggcaaaagtcaccattgatggtgatatcaaactgcttggtgaaaacgagtccatttatattccgctgggggcgacgcattgcctggaaaacccggggaaaattccgctcgatttaattgaagtgcgctccggctcttatctcgaagaggatgatgtggtgcgtttcgcggatcgctacggacgggtgtaa

SEQ ID NO: 32 - protein, 478 aa, Homo sapiens
MAQSKLYPVVMAGGSGSRLWPLSRVLYPKQFLCLKGDLTMLQTTICRLNGVECESPVVICNEQHRFIVAEQLRQLNKLTENIILEPAGRNTAPAIALAALAAKRHSPESDPLMLVLAADHVIADEDAFRAAVRNAMPYAEAGKLVTFGIVPDLPETGYGYIRRGEVSAGEQDMVAFEVAQFVEKPNLETAQAYVASGEYYWNSGMFLFRAGRYLEELKKYRPDILDACEKAMSAVDPDLNFIRVDEEAFLACPEESVDYAVMERTADAVVVPMDAGWSDVGSWSSLWEISAHTAEGNVCHGDVINHKTENSYVYAESGLVTTVGVKDLVVVQTKDAVLIADRNAVQDVKKVVEQIKADGRHEHRVHREVYRPWGKYDSIDAGDRYQVKRITVKPGEGLSVQMHHHRAEHWVVVAGTAKVTIDGDIKLLGENESIYIPLGATHCLENPGKIPLDLIEVRSGSYLEEDDVVRFADRYGRV

SEQ ID NO: 33 - DNA, 1122 bp, Homo sapiens
  source 1..1122: mol_type = genomic DNA
atgtcaaaagtcgctctcatcaccggtgtaaccggacaagacggttcttacctggcagagtttctgctggaaaaaggttacgaggtgcatggtattaagcgtcgcgcatcgtcattcaacaccgagcgcgtggatcacatttatcaggatccgcacacctgcaacccgaaattccatctgcattatggcgacctgagtgatacctctaacctgacgcgcattttgcgtgaagtacagccggatgaagtgtacaacctgggcgcaatgagccacgttgcggtctcttttgagtcaccagaatataccgctgacgtcgacgcgatgggtacgctgcgcctgctggaggcgatccgcttcctcggtctggaaaagaaaactcgtttctatcaggcttccacctctgaactgtatggtctggtgcaggaaattccgcagaaagagaccacgccgttctacccgcgatctccgtatgcggtcgccaaactgtacgcctactggatcaccgttaactaccgtgaatcctacggcatgtacgcctgtaacggaattctcttcaaccatgaatccccgcgccgcggcgaaaccttcgttacccgcaaaatcacccgcgcaatcgccaacatcgcccaggggctggagtcgtgcctgtacctcggcaatatggattccctgcgtgactggggccacgccaaagactacgtaaaaatgcagtggatgatgctgcagcaggaacagccggaagatttcgttatcgcgaccggcgttcagtactccgtgcgtcagttcgtggaaatggcggcagcacagctgggcatcaaactgcgctttgaaggcacgggcgttgaagagaagggcattgtggtttccgtcaccgggcatgacgcgccgggcgttaaaccgggtgatgtgattatcgctgttgacccgcgttacttccgtccggctgaagttgaaacgctgctcggcgacccgaccaaagcgcacgaaaaactgggctggaaaccggaaatcaccctcagagagatggtgtctgaaatggtggctaatgacctcgaagcggcgaaaaaacactctctgctgaaatctcacggctacgacgtggcgatcgcgctggagtcataa

SEQ ID NO: 34 - protein, 373 aa, Homo sapiens [sequence omitted]

SEQ ID NO: 35 - DNA, 966 bp, Homo sapiens
  source 1..966: mol_type = genomic DNA
atgagtaaacaacgagtttttattgctggtcatcgcgggatggtcggttccgccatcaagcgacagctcgaacagcgcggtgatgtggagctggtattacgcacccgcgatgaactgaacctgctggacagccgcgcggtgcatgatttctttgccagcgaaagcattgaccaggtctatctggcggcggcgaaagtgggcggcattgttgccaacaacacgtatccggcggatttcatctaccagaacatgatgattgagagcaacatcattcacgccgcgcatcagaacgacgtgaacaaactgctgtttctcggatcgtcctgtatctacccgaaactggcaaaacagccgatggcagaaagcgagttattgcagggcacgctggagccgaccaacgagccttatgccattgccaaaattgccgggatcaaactgtgcgaatcttacaaccgccagtacggacgcgattaccgctcagtcatgccgaccaacctgtatggcccgcatgacaacttccacccgagtaattcgcatgtgatcccagcattgttgcgtcgcttccacgaggcgacggcacagaatgcgccggacgttgtggtatggggcagcggtacaccgatgcgtgaattcctgcacgtcgatgatatggcggcggccagcattcacgtgatggagctggcgcacgaagtctggctggagaacacccagccgatgctgtcgcacattaacgtcggtaccggtgttgactgcactatccgcgagctggcgcaaaccatcgccaaagtggtggattacaaaggtcgggtggtttttgatgccagcaagccggatggtacgccgcgcaaactgctggatgtgacgcgcctgcatcagcttggctggtatcacgaaatctcactggaagcggggcttgccagcacttaccagtggttccttgagaatcgagaccgctttcgggggtaa

SEQ ID NO: 36 - protein, 321 aa, Homo sapiens
MSKQRVFIAGHRGMVGSAIKRQLEQRGDVELVLRTRDELNLLDSRAVHDFFASESIDQVYLAAAKVGGIVANNTYPADFIYQNMMIESNIIHAAHQNDVNKLLFLGSSCIYPKLAKQPMAESELLQGTLEPTNEPYAIAKIAGIKLCESYNRQYGRDYRSVMPTNLYGPHDNFHPSNSHVIPALLRRFHEATAQNAPDVVVWGSGTPMREFLHVDDMAAASIHVMELAHEVWLENTQPMLSHINVGTGVDCTIRELAQTIAKVVDYKGRVVFDASKPDGTPRKLLDVTRLHQLGWYHEISLEAGLASTYQWFLENRDRFRG

SEQ ID NO: 37 - DNA, 1830 bp, Homo sapiens
  source 1..1830: mol_type = genomic DNA
atgtgtggaattgttggcgcgatcgcgcaacgtgatgtagcagaaatccttcttgaaggtttacgtcgtctggaataccgcggatatgactctgccggtctggccgttgttgatgcagaaggtcatatgacccgcctgcgtcgcctcggtaaagtccagatgctggcacaggcagcggaagaacatcctctgcatggcggcactggtattgctcacactcgctgggcgacccacggtgaaccttcagaagtgaatgcgcatccgcatgtttctgaacacattgtggtggtgcataacggcatcatcgaaaaccatgaaccgctgcgtgaagagctaaaagcgcgtggctataccttcgtttctgaaaccgacaccgaagtgattgcccatctggtgaactgggagctgaaacaaggcgggactctgcgtgaggccgttctgcgtgctatcccgcagctgcgtggtgcgtacggtacagtgatcatggactcccgtcacccggataccctgctggcggcacgttctggtagtccgctggtgattggcctggggatgggcgaaaactttatcgcttctgaccagctggcgctgttgccggtgacccgtcgctttatcttccttgaagagggcgatattgcggaaatcactcgccgttcggtaaacatcttcgataaaactggcgcggaagtaaaacgtcaggatatcgaatccaatctgcaatatgacgcgggcgataaaggcatttaccgtcactacatgcagaaagagatctacgaacagccgaacgcgatcaaaaacacccttaccggacgcatcagccacggtcaggttgatttaagcgagctgggaccgaacgccgacgaactgctgtcgaaggttgagcatattcagatcctcgcctgtggtacttcttataactccggtatggtttcccgctactggtttgaatcgctagcaggtattccgtgcgacgtcgaaatcgcctctgaattccgctatcgcaaatctgccgtgcgtcgtaacagcctgatgatcaccttgtcacagtctggcgaaaccgcggataccctggctggcctgcgtctgtcgaaagagctgggttaccttggttcactggcaatctgtaacgttccgggttcttctctggtgcgcgaatccgatctggcgctaatgaccaacgcgggtacagaaatcggcgtggcatccactaaagcattcaccactcagttaactgtgctgttgatgctggtggcgaagctgtctcgcctgaaaggtctggatgcctccattgaacatgacatcgtgcatggtctgcaggcgctgccgagccgtattgagcagatgctgtctcaggacaaacgcattgaagcgctggcagaagatttctctgacaaacatcacgcgctgttcctgggccgtggcgatcagtacccaatcgcgctggaaggcgcattgaagttgaaagagatctcttacattcacgctgaagcctacgctgctggcgaactgaaacacggtccgctggcgctaattgatgccgatatgccggttattgttgttgcaccgaacaacgaattgctggaaaaactgaaatccaacattgaagaagttcgcgcgcgtggcggtcagttgtatgtcttcgccgatcaggatgcgggttttgtaagtagcgataacatgcacatcatcgagatgccgcatgtggaagaggtgattgcaccgatcttctacaccgttccgctgcagctgctggcttaccatgtcgcgctgatcaaaggcaccgacgttgaccagccgcgtaacctggcaaaatcggttacggttgagtaa

SEQ ID NO: 38 - protein, 609 aa, Homo sapiens
MCGIVGAIAQRDVAEILLEGLRRLEYRGYDSAGLAVVDAEGHMTRLRRLGKVQMLAQAAEEHPLHGGTGIAHTRWATHGEPSEVNAHPHVSEHIVVVHNGIIENHEPLREELKARGYTFVSETDTEVIAHLVNWELKQGGTLREAVLRAIPQLRGAYGTVIMDSRHPDTLLAARSGSPLVIGLGMGENFIASDQLALLPVTRRFIFLEEGDIAEITRRSVNIFDKTGAEVKRQDIESNLQYDAGDKGIYRHYMQKEIYEQPNAIKNTLTGRISHGQVDLSELGPNADELLSKVEHIQILACGTSYNSGMVSRYWFESLAGIPCDVEIASEFRYRKSAVRRNSLMITLSQSGETADTLAGLRLSKELGYLGSLAICNVPGSSLVRESDLALMTNAGTEIGVASTKAFTTQLTVLLMLVAKLSRLKGLDASIEHDIVHGLQALPSRIEQMLSQDKRIEALAEDFSDKHHALFLGRGDQYPIALEGALKLKEISYIHAEAYAAGELKHGPLALIDADMPVIVVAPNNELLEKLKSNIEEVRARGGQLYVFADQDAGFVSSDNMHIIEMPHVEEVIAPIFYTVPLQLLAYHVALIKGTDVDQPRNLAKSVTVE

SEQ ID NO: 39 - DNA, 1338 bp, Homo sapiens
  source 1..1338: mol_type = genomic DNA
atgagtaatcgtaaatatttcggtaccgatgggattcgtggtcgtgtaggggatgcgccgatcacacctgattttgtgcttaagctgggttgggccgcgggtaaagtgctggcgcgccacggctcccgtaagattattattggtaaagacacgcgtatttctggctatatgctggagtcagcactggaagcgggtctggcggcagcgggcctttccgcactcttcactggcccgatgccaacaccggccgtggcttatctgacgcgtaccttccgcgcagaggccggaattgtgatatctgcatcgcataacccgttctacgataatggcattaaattcttctctatcgacggcaccaaactgccggatgcggtagaagaggccatcgaagcggaaatggaaaaggagatcagctgcgttgattcggcagaactgggtaaagccagccgtatcgttgatgccgcgggtcgctatatcgagttttgcaaagccacgttcccgaacgaacttagcctcagtgaactgaagattgtggtggattgtgcaaacggtgcgacttatcacatcgcgccgaacgtgctgcgcgaactgggggcgaacgttatcgctatcggttgtgagccaaacggtgtaaacatcaatgccgaagtgggggctaccgacgttcgcgcgctccaggctcgtgtgctggctgaaaaagcggatctcggtattgccttcgacggcgatggcgatcgcgtgattatggttgaccatgaaggcaataaagtcgatggcgatcagatcatgtatatcatcgcgcgtgaaggtcttcgtcagggccagctgcgtggtggcgctgtgggtacattgatgagcaacatggggcttgaactggcgctgaaacagttaggaattccatttgcgcgcgcgaaagtgggtgaccgctacgtactggaaaaaatgcaggagaaaggctggcgtatcggtgcagagaattccggtcatgtgatcctgctggataaaactactaccggtgacggcatcgttgctggcttgcaggtgctggcggcgatggcacgtaaccatatgagcctgcacgacctttgcagcggcatgaaaatgttcccgcagattctggttaacgtacgttacaccgcaggtagcggcgatccacttgagcatgagtcagttaaagccgtgaccgcagaggttgaagctgcgctgggcaaccgtggacgcgtgttgctgcgtaaatccggcaccgaaccgttaattcgcgtgatggtggaaggcgaagacgaagcgcaggtgactgaatttgcacaccgcatcgccgatgcagtaaaagccgtttaa

SEQ ID NO: 40 - protein, 445 aa, Homo sapiens
MSNRKYFGTDGIRGRVGDAPITPDFVLKLGWAAGKVLARHGSRKIIIGKDTRISGYMLESALEAGLAAAGLSALFTGPMPTPAVAYLTRTFRAEAGIVISASHNPFYDNGIKFFSIDGTKLPDAVEEAIEAEMEKEISCVDSAELGKASRIVDAAGRYIEFCKATFPNELSLSELKIVVDCANGATYHIAPNVLRELGANVIAIGCEPNGVNINAEVGATDVRALQARVLAEKADLGIAFDGDGDRVIMVDHEGNKVDGDQIMYIIAREGLRQGQLRGGAVGTLMSNMGLELALKQLGIPFARAKVGDRYVLEKMQEKGWRIGAENSGHVILLDKTTTGDGIVAGLQVLAAMARNHMSLHDLCSGMKMFPQILVNVRYTAGSGDPLEHESVKAVTAEVEAALGNRGRVLLRKSGTEPLIRVMVEGEDEAQVTEFAHRIADAVKAV

SEQ ID NO: 41 - DNA, 1371 bp, Homo sapiens
  source 1..1371: mol_type = genomic DNA
atgttgaataatgctatgagcgtagtgatccttgccgcaggcaaaggcacgcgcatgtattccgatcttccgaaagtgctgcatacccttgccgggaaagcgatggttcagcatgtcattgatgctgcgaatgaattaggcgcagcgcacgttcacctggtgtacggtcacggcggcgatctgctaaaacaggcgctgaaagacgacaaccttaactgggtgcttcaggcagagcagctgggtacgggtcatgcaatgcagcaggccgcacctttctttgccgatgatgaagacattttaatgctctacggcgacgtgccgctgatctctgtcgaaacactccagcgtctgcgtgatgctaaaccgcagggtggcattggtctgctgacggtgaaactggatgatccgaccggttatggacgtatcacccgtgaaaacggcaaagttaccggcattgttgagcacaaagatgccaccgacgagcagcgtcagattcaggagatcaacaccggcattctgattgccaacggcgcagatatgaaacgctggctggcgaagctgaccaacaataatgctcagggcgaatactacatcaccgacattattgcgctggcgtatcaggaagggcgtgaaatcgtcgccgttcatccgcaacgtttaagcgaagtagaaggcgtgaataaccgcctgcaactctcccgtctggagcgtgtttatcagtccgaacaggctgaaaaactgctgttagcaggcgttatgctgcgcgatccagcgcgttttgatctgcgtggtacgctaactcacgggcgcgatgttgaaattgatactaacgttatcatcgagggcaacgtgactctcggtcatcgcgtgaaaattggcaccggttgcgtgattaaaaacagcgtgattggcgatgattgcgaaatcagtccgtataccgttgtggaagatgcgaatctggcagcggcctgtaccattggcccgtttgcccgtttgcgtcctggtgctgagttgctggaaggtgctcacgtcggtaacttcgttgagatgaaaaaagcgcgtctgggtaaaggctcgaaagctggtcatctgacttacctgggcgatgcggaaattggcgataacgttaacatcggcgcgggaaccattacctgcaactacgatggtgcgaataaatttaagaccattatcggcgacgatgtgtttgttggttccgacactcagctggtggccccggtaacagtaggcaaaggcgcgaccattgctgcgggtacaactgtgacgcgtaatgtcggcgaaaatgcattagctatcagccgtgtgccgcagactcagaaagaaggctggcgtcgtccggtaaagaaaaagtga

SEQ ID NO: 42 - protein, 456 aa, Homo sapiens
MLNNAMSVVILAAGKGTRMYSDLPKVLHTLAGKAMVQHVIDAANELGAAHVHLVYGHGGDLLKQALKDDNLNWVLQAEQLGTGHAMQQAAPFFADDEDILMLYGDVPLISVETLQRLRDAKPQGGIGLLTVKLDDPTGYGRITRENGKVTGIVEHKDATDEQRQIQEINTGILIANGADMKRWLAKLTNNNAQGEYYITDIIALAYQEGREIVAVHPQRLSEVEGVNNRLQLSRLERVYQSEQAEKLLLAGVMLRDPARFDLRGTLTHGRDVEIDTNVIIEGNVTLGHRVKIGTGCVIKNSVIGDDCEISPYTVVEDANLAAACTIGPFARLRPGAELLEGAHVGNFVEMKKARLGKGSKAGHLTYLGDAEIGDNVNIGAGTITCNYDGANKFKTIIGDDVFVGSDTQLVAPVTVGKGATIAAGTTVTRNVGENALAISRVPQTQKEGWRRPVKKK

SEQ ID NO: 43 - DNA, 2169 bp, Homo sapiens
  source 1..2169: mol_type = genomic DNA
atggagaagaacgggaacaaccgaaagctccgggtttgcgttgccacctgcaaccgagctgactactccaaactggccccgatcatgttcggcatcaagacagagcccgcgttctttgagttggacgtggtggtgctcggctcccacctgattgacgactatggaaacacataccgcatgattgagcaagatgactttgacattaacaccaggctccacacgattgttagaggggaagatgaagcggccatggtagagtcggtaggcctagcgctcgtgaagctaccggacgtcctcaatcgcctgaagcccgacatcatgattgttcacggagatcgatttgacgcccttgctctggctacgtctgctgccttgatgaacatccgcatccttcacattgaaggaggcgaggtcagcgggaccattgatgactctatcagacacgccataacaaaactggctcactaccatgtgtgctgcactagaagtgcagagcagcacctgatctctatgtgcgaggaccacgaccgcatcctgttggcaggctgcccttcctatgacaaactgctctccgccaagaacaaagactatatgagcatcattcggatgtggctaggcgatgatgtaaaatgtaaggattacatcgttgccctgcagcatcccgtgaccactgacattaagcattccataaagatgtttgagctaacactggatgccctgatctcgtttaacaagaggaccctagttctgtttccaaatatcgatgcaggcagcaaggagatggttcgagtgatgcggaagaagggcatcgagcatcaccccaatttccgtgcagtcaagcacgtcccgtttgaccagttcatacagctggtcgcccacgctggctgcatgattgggaatagcagctgcggcgtgcgagaggttggcgctttcggaacacccgtgatcaacctgggcacaaggcagataggaagagaaaccggggagaatgttcttcatgtcagggatgctgacacccaagataaaatattgcaagcactacacctccagttcggcaaacagtacccttgctcaaagatatatggggatgggaatgctgttccaaggattttaaagtttctcaaatccattgaccttcaagagccactacagaagaaattctgcttcccccctgtaaaggagaacatctctcaagacattgaccacatcctggaaactctgagtgccttggctgttgatcttggcgggacaaacctgagggtggcaatagttagcatgaagggtgaaatcgttaagaagtacactcagttcaaccctaaaacctatgaagaaaggattagtttaatcctgcagatgtgtgtggaagctgccgcggaagctgtgaaactcaattgcagaatactgggagtaggcatctccacaggtggccgcgtgaatccccaggaaggagttgtgctgcattcaaccaagctgatccaggaatggaactccgtggacctcaggacacccctctccgacaccctgcatctccccgtgtgggtggacaatgacggcaactgtgccgccatggcagagaggaagttcggccaaggaaaaggacaggagaacttcgtgacgctcatcacggggacagggatcggtggggggatcatccaccagcacgaactgatccacggcagctccttctgcgcggcggagctcggccatctcgtggtgtccctggacggtcctgactgctcctgtggaagccatgggtgcatcgaagcgtacgcctctggaatggccttgcagagggaagcaaagaaactccatgatgaggacctgctcttggtggaagggatgtcagtaccaaaagacgaagctgtgggtgccctccatctcatccaggctgccaagctgggcaacgtgaaggcccagagcatcttacgaacagctggaactgctttgggacttggggttgtgaacatcctccacactatgaatccttccctggtgatcctgtctggagtcctggccagtcactacatccacatcgtcaaggacgtcatccgccagcaagccttgtcctccgtgcaggatgtggacgtggtggtgtcagacttggtggacccggccctgcttggcgcagccagcatggttctggactacacaacgcgcaggatacactag

SEQ ID NO: 44 - protein, 722 aa, Homo sapiens
MEKNGNNRKLRVCVATCNRADYSKLAPIMFGIKTEPAFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKCKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERISLILQMCVEAAAEAVKLNCRILGVGISTGGRVNPQEGVVLHSTKLIQEWNSVDLRTPLSDTLHLPVWVDNDGNCAAMAERKFGQGKGQENFVTLITGTGIGGGIIHQHELIHGSSFCAAELGHLVVSLDGPDCSCGSHGCIEAYASGMALQREAKKLHDEDLLLVEGMSVPKDEAVGALHLIQAAKLGNVKAQSILRTAGTALGLGVVNILHTMNPSLVILSGVLASHYIHIVKDVIRQQALSSVQDVDVVVSDLVDPALLGAASMVLDYTTRRIH

SEQ ID NO: 45 - DNA, 1080 bp, Homo sapiens
  source 1..1080: mol_type = genomic DNA
atgccgctggagctggagctgtgtcccgggcgctgggtgggaggtcaacacccgtgcttcatcattgccgagatcggccagaaccaccagggcgacctggacgtagccaagcgcatgatccgcatggccaaggagtgtggggctgattgtgctaagttccagaagagtgagctagaatttaagtttaatcggaaagccttggagaggccatacacctcgaagcattcctgggggaaaacgtacggggagcacaaacgacatctggagttcagccatgaccagtacagggagctgcagaggtacgccgaggaggttgggatcttcttcactgcctctggcatggatgagatggcagttgaatttctgcatgaactgaatgttccatttttcaaagttggatctggagacactaataattttccttatctggaaaagacagccaaaaaaggtcgcccaatggtgatctccagtgggatgcagtcaatggacaccatgaagcaagtttatcagatcgtgaagcccctcaaccccaacttctgcttcttgcagtgtaccagcgcatacccgctccagcctgaggacgtcaacctgcgggtcatctcggaatatcagaagctctttcctgacattcccatagggtattctgggcatgaaacaggcatagcgatatctgtggccgcagtggctctgggggccaaggtgttggaacgtcacataactttggacaagacctggaaggggagtgaccactcggcctcgctggagcctggagaactggccgagctggtgcggtcagtgcgtcttgtggagcgtgccctgggctccccaaccaagcagctgctgccctgtgagatggcctgcaatgagaagctgggcaagtctgtggtggccaaagtgaaaattccggaaggcaccattctaacaatggacatgctcaccgtgaaggtgggtgagcccaaaggctatcctcctgaggacatctttaatctagtgggcaagaaggtcctggtcactgttgaagaggatgacaccatcatggaagaattggtagataatcatggcaaaaaaatcaagtcttaa

SEQ ID NO: 46 - protein, 359 aa, Homo sapiens
MPLELELCPGRWVGGQHPCFIIAEIGQNHQGDLDVAKRMIRMAKECGADCAKFQKSELEFKFNRKALERPYTSKHSWGKTYGEHKRHLEFSHDQYRELQRYAEEVGIFFTASGMDEMAVEFLHELNVPFFKVGSGDTNNFPYLEKTAKKGRPMVISSGMQSMDTMKQVYQIVKPLNPNFCFLQCTSAYPLQPEDVNLRVISEYQKLFPDIPIGYSGHETGIAISVAAVALGAKVLERHITLDKTWKGSDHSASLEPGELAELVRSVRLVERALGSPTKQLLPCEMACNEKLGKSVVAKVKIPEGTILTMDMLTVKVGEPKGYPPEDIFNLVGKKVLVTVEEDDTIMEELVDNHGKKIKS

SEQ ID NO: 47 - DNA, 747 bp, Homo sapiens
  source 1..747: mol_type = genomic DNA
atggggctgagccgcgtgcgggcggttttctttgacttggacaacactctcatcgacacggccggggcgagcaggagaggcatgttggaggtgataaaactcttacaatcaaaataccattataaagaagaggctgaaatcatctgtgataaagttcaagttaaactcagcaaggaatgttttcatccttacaatacatgcattactgatttaaggacttcacattgggaagaagcaatccaggaaacaaaaggtggtgcagccaatagaaaattggctgaagaatgttatttcctttggaaatctacacgtttacagcacatgacactagcagaagatgtcaaagccatgcttactgaacttcgaaaggaggtccgcctacttctattaacgaatggggacagacagacccagagggagaagattgaggcttgtgcctgtcagtcctattttgacgctgttgttgtaggtggagagcagagagaggagaaaccagcaccgtccatattttattactgctgcaatcttctcggagtacaacctggggactgtgtgatggtcggtgacacattagaaaccgacatccaaggaggcctcaatgcaggattgaaagcaacagtctggatcaataaaaatggaatagtgccactgaagtcctccccagttccgcattacatggtttcttctgtgctagagttacctgctctcttacaaagtatagactgcaaagtcagtatgtccacttaa

SEQ ID NO: 48 - protein, 248 aa, Homo sapiens
MGLSRVRAVFFDLDNTLIDTAGASRRGMLEVIKLLQSKYHYKEEAEIICDKVQVKLSKECFHPYNTCITDLRTSHWEEAIQETKGGAANRKLAEECYFLWKSTRLQHMTLAEDVKAMLTELRKEVRLLLLTNGDRQTQREKIEACACQSYFDAVVVGGEQREEKPAPSIFYYCCNLLGVQPGDCVMVGDTLETDIQGGLNAGLKATVWINKNGIVPLKSSPVPHYMVSSVLELPALLQSIDCKVSMST

SEQ ID NO: 49 - DNA, 1305 bp, Homo sapiens
  source 1..1305: mol_type = genomic DNA
atggactcggtggagaagggtgctgctacatcagtgtccaacccgcgtggacgaccatcccgaggccggcctcctaagctgcagcgaaactctcgcggcggccagggccgaggtgtggagaagcccccgcacctggcagccctaattctggcccggggaggcagcaaaggcatccccctgaagaacattaagcacctggcgggggtcccgctcattggctgggtcctgcgtgcggccctggattcaggggccttccagagtgtatgggtttcgacagaccatgatgaaattgagaatgtggccaaacaatttggtgcacaagttcatcgaagaagttctgaagtttcaaaagacagctctacctcactagatgccatcatagaatttcttaattatcataatgaggttgacattgtaggaaatattcaagctacttctccatgtttacatcctactgatcttcaaaaagttgcagaaatgattcgagaagaaggatatgattctgttttctctgttgtgaggcgccatcagtttcgatggagtgaaattcagaaaggagttcgtgaagtgaccgaacctctgaatttaaatccagctaaacggcctcgtcgacaagactgggatggagaattatatgaaaatggctcattttattttgctaaaagacatttgatagagatgggttacttgcagggtggaaaaatggcatactacgaaatgcgagctgaacatagtgtggatatagatgtggatattgattggcctattgcagagcaaagagtattaagatatggctattttggcaaagagaagcttaaggaaataaaacttttggtttgcaatattgatggatgtctcaccaatggccacatttatgtatcaggtgaccaaaaagaaataatatcttatgatgtaaaagatgctattgggataagtttattaaagaaaagtggtattgaggtgaggctaatctcagaaagggcctgttcaaagcagacgctgtcatctttaaaactggattgcaaaatggaagtcagtgtatcagacaagctagcagttgtagatgaatggagaaaagaaatgggcctgtgctggaaagaagtggcatatcttggaaatgaagtgtctgatgaagagtgcttgaagagagtgggcctaagtggcgctcctgctgatgcctgttctactgcccagaaggctgttggatacatttgcaaatgtaatggtggccgtggtgccatccgagaatttgcagagcacatttgcctactaatggaaaaggttaataattcatgccaaaaatag

SEQ ID NO: 50 - protein, 434 aa, Homo sapiens
MDSVEKGAATSVSNPRGRPSRGRPPKLQRNSRGGQGRGVEKPPHLAALILARGGSKGIPLKNIKHLAGVPLIGWVLRAALDSGAFQSVWVSTDHDEIENVAKQFGAQVHRRSSEVSKDSSTSLDAIIEFLNYHNEVDIVGNIQATSPCLHPTDLQKVAEMIREEGYDSVFSVVRRHQFRWSEIQKGVREVTEPLNLNPAKRPRRQDWDGELYENGSFYFAKRHLIEMGYLQGGKMAYYEMRAEHSVDIDVDIDWPIAEQRVLRYGYFGKEKLKEIKLLVCNIDGCLTNGHIYVSGDQKEIISYDVKDAIGISLLKKSGIEVRLISERACSKQTLSSLKLDCKMEVSVSDKLAVVDEWRKEMGLCWKEVAYLGNEVSDEECLKRVGLSGAPADACSTAQKAVGYICKCNGGRGAIREFAEHICLLMEKVNNSCQK